SEQUENCE LISTING (96 sequences)

SEQ ID NO: 1 - protein, 10 aa, Artificial
  CDR sequence of Claudin 18.2 VHH
GDRFSSYAMG

SEQ ID NO: 2 - protein, 17 aa, Artificial
  CDR sequence of Claudin 18.2 VHH
AVSWSGRITDYADSVKG

SEQ ID NO: 3 - protein, 9 aa, Artificial
  CDR sequence of Claudin 18.2 VHH
YANLLWRDH

SEQ ID NO: 4 - protein, 10 aa, Artificial
  CDR sequence of Claudin 18.2 VHH
GFTFSNYAMY

SEQ ID NO: 5 - protein, 17 aa, Artificial
  CDR sequence of Claudin 18.2 VHH
VISGGGGVISYADSVKG

SEQ ID NO: 6 - protein, 12 aa, Artificial
  CDR sequence of Claudin 18.2 VHH
NAQSAVWWENDY

SEQ ID NO: 7 - protein, 10 aa, Artificial
  CDR sequence of Claudin 18.2 VHH
GLTFSLYDMG

SEQ ID NO: 8 - protein, 16 aa, Artificial
  CDR sequence of Claudin 18.2 VHH
TITSSGNTGYAESVKG

SEQ ID NO: 9 - protein, 6 aa, Artificial
  CDR sequence of Claudin 18.2 VHH
YVPFLG

SEQ ID NO: 10 - protein, 10 aa, Artificial
  CDR sequence of Claudin 18.2 VHH
GAALSSYTMG

SEQ ID NO: 11 - protein, 17 aa, Artificial
  CDR sequence of Claudin 18.2 VHH
AISWGGSITWYADSVKG

SEQ ID NO: 12 - protein, 15 aa, Artificial
  CDR sequence of Claudin 18.2 VHH
HAEGQFGALSYPKDY

SEQ ID NO: 13 - protein, 10 aa, Artificial
  CDR sequence of CD3 VH
GYTFTSYTMH

SEQ ID NO: 14 - protein, 17 aa, Artificial
  CDR sequence of CD3 VH
YINPSSGYTKYNQKFKD

SEQ ID NO: 15 - protein, 12 aa, Artificial
  CDR sequence of CD3 VH
ARWQDYDVYFDY

SEQ ID NO: 16 - protein, 10 aa, Artificial
  CDR sequence of CD3 VL
RASSSVSYIH

SEQ ID NO: 17 - protein, 7 aa, Artificial
  CDR sequence of CD3 VL
ATSNLAS

SEQ ID NO: 18 - protein, 9 aa, Artificial
  CDR sequence of CD3 VL
QQWTSNPPT

SEQ ID NO: 19 - protein, 116 aa, Artificial
  Claudin 18.2 VHH sequence
QVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSS

SEQ ID NO: 20 - protein, 119 aa, Artificial
  Claudin 18.2 VHH sequence
QVQLVESGGGLVQPGGSLRLSCAASGFTFSNYAMYWVRQAPGKGPEWVSVISGGGGVISYADSVKGRFTISRDNAKNTMYLQMNSLKPEDTAVYYCNAQSAVWWENDYWGQGTQVTVSS

SEQ ID NO: 21 - protein, 112 aa, Artificial
  Claudin 18.2 VHH sequence
QVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSS

SEQ ID NO: 22 - protein, 122 aa, Artificial
  Claudin 18.2 VHH sequence
QVQLVESGGGVVQTGGSLRLTCEISGAALSSYTMGWFRQAPGKEREFVAAISWGGSITWYADSVKGRFTISRDNAKNTVYLQMNSLKPEDTAVYYCHAEGQFGALSYPKDYWGQGTQVTVSS

SEQ ID NO: 23 - protein, 119 aa, Artificial
  CD3 VH sequence
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSS

SEQ ID NO: 24 - protein, 106 aa, Artificial [sequence omitted]

SEQ ID NO: 25 - protein, 15 aa, Homo sapiens
EPKSCDKTHTCPPCP

SEQ ID NO: 26 - protein, 15 aa, Artificial
  Fc hinge sequence
GGGGSDKTHTCPPCP

SEQ ID NO: 27 - protein, 15 aa, Artificial
  Fc hinge sequence
EPKSSDKTHTCPPCP

SEQ ID NO: 28 - protein, 10 aa, Artificial
  Fc hinge sequence
DKTHTCPPCP

SEQ ID NO: 29 - protein, 16 aa, Artificial
  Fc hinge sequence
STMVRSDKTHTCPPCP

SEQ ID NO: 30 - protein, 217 aa, Artificial
  Fc region sequence with knob mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 217 aa, Artificial
  Fc region sequence hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 32 - protein, 217 aa, Artificial
  Wildtype IgG1 Fc region
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 34 - protein, 107 aa, Homo sapiens [sequence omitted]

SEQ ID NO: 35 - protein, 5 aa, Artificial
  Linker peptide
GGGGS

SEQ ID NO: 36 - protein, 10 aa, Artificial
  Linker peptide
GGGGSGGGGS

SEQ ID NO: 37 - protein, 15 aa, Artificial
  Linker peptide
GGGGSGGGGSGGGGS

SEQ ID NO: 38 - protein, 20 aa, Artificial
  Linker peptide
GGGGSGGGGSGGGGSGGGGS

SEQ ID NO: 39 - protein, 25 aa, Artificial
  Linker peptide
GGGGSGGGGSGGGGSGGGGSGGGGS

SEQ ID NO: 40 - protein, 30 aa, Artificial
  Linker peptide
GGGGSGGGGSGGGGSGGGGSGGGGSGGGGS

SEQ ID NO: 41 - protein, 348 aa, Artificial
  Exemplary chain 1 sequence of Claudin 18.2/CD3 BsAb in BiSpecific-V1 format
QVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 348 aa, Artificial
  Exemplary chain 1 sequence of Claudin 18.2/CD3 BsAb in BiSpecific-V1 format
QVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 449 aa, Artificial
  Exemplary chain 2 sequence of Claudin 18.2/CD3 BsAb in BiSpecific-V1 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 44 - protein, 449 aa, Artificial
  Exemplary chain 2 sequence of Claudin 18.2/CD3 BsAb in BiSpecific-V1 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 45 - protein, 213 aa, Artificial
  Exemplary chain 3 sequence of Claudin 18.2/CD3 BsAb in BiSpecific-V1 format
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 482 aa, Artificial
  Exemplary chain 1 sequence of Claudin 18.2/CD3 TsAb in TriSpecific-V1 format
QVQLVESGGGLVQPGGSLRLSCAASGFTFSNYAMYWVRQAPGKGPEWVSVISGGGGVISYADSVKGRFTISRDNAKNTMYLQMNSLKPEDTAVYYCNAQSAVWWENDYWGQGTQVTVSSGGGGSGGGGSGGGGSQVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 477 aa, Artificial
  Exemplary chain 1 sequence of Claudin 18.2/CD3 TsAb in TriSpecific-V1 format
QVQLVESGGGLVQPGGSLRLSCAASGFTFSNYAMYWVRQAPGKGPEWVSVISGGGGVISYADSVKGRFTISRDNAKNTMYLQMNSLKPEDTAVYYCNAQSAVWWENDYWGQGTQVTVSSGGGGSGGGGSQVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 449 aa, Artificial; identical to SEQ ID NO: 43
  Exemplary chain 2 sequence of Claudin 18.2/CD3 TsAb in TriSpecific-V1 format

SEQ ID NO: 49 - protein, 449 aa, Artificial; identical to SEQ ID NO: 44
  Exemplary chain 2 sequence of Claudin 18.2/CD3 TsAb in TriSpecific-V1 format

SEQ ID NO: 50 - protein, 213 aa, Artificial; identical to SEQ ID NO: 45
  Exemplary chain 3 sequence of Claudin 18.2/CD3 TsAb in TriSpecific-V1 format

SEQ ID NO: 51 - protein, 348 aa, Artificial; identical to SEQ ID NO: 41
  Exemplary chain 1 sequence of Claudin 18.2/CD3 BsAb in BiSpecific-V2 format

SEQ ID NO: 52 - protein, 348 aa, Artificial; identical to SEQ ID NO: 42
  Exemplary chain 1 sequence of Claudin 18.2/CD3 BsAb in BiSpecific-V2 format

SEQ ID NO: 53 - protein, 472 aa, Artificial
  Exemplary chain 2 sequence of Claudin 18.2/CD3 BsAb in BiSpecific-V2 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 54 - protein, 472 aa, Artificial
  Exemplary chain 2 sequence of Claudin 18.2/CD3 BsAb in BiSpecific-V2 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 55 - protein, 482 aa, Artificial; identical to SEQ ID NO: 46
  Exemplary chain 1 sequence of Claudin 18.2/CD3 TsAb in TriSpecific-V2 format

SEQ ID NO: 56 - protein, 477 aa, Artificial; identical to SEQ ID NO: 47
  Exemplary chain 1 sequence of Claudin 18.2/CD3 TsAb in TriSpecific-V2 format

SEQ ID NO: 57 - protein, 472 aa, Artificial; identical to SEQ ID NO: 53
  Exemplary chain 2 sequence of Claudin 18.2/CD3 TsAb in TriSpecific-V2 format

SEQ ID NO: 58 - protein, 472 aa, Artificial; identical to SEQ ID NO: 54
  Exemplary chain 2 sequence of Claudin 18.2/CD3 TsAb in TriSpecific-V2 format

SEQ ID NO: 59 - protein, 580 aa, Artificial
  Exemplary chain 1 sequence of Claudin 18.2/CD3 BsAb in BiSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSS

SEQ ID NO: 60 - protein, 575 aa, Artificial
  Exemplary chain 1 sequence of Claudin 18.2/CD3 BsAb in BiSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSS

SEQ ID NO: 61 - protein, 213 aa, Artificial; identical to SEQ ID NO: 45
  Exemplary chain 2 sequence of Claudin 18.2/CD3 BsAb in BiSpecific-V3 format

SEQ ID NO: 62 - protein, 580 aa, Artificial
  Exemplary chain 1 sequence of Claudin 18.2/CD3 TsAb in TriSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSS

SEQ ID NO: 63 - protein, 575 aa, Artificial
  Exemplary chain 1 sequence of Claudin 18.2/CD3 TsAb in TriSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSS

SEQ ID NO: 64 - protein, 583 aa, Artificial
  Exemplary chain 2 sequence of Claudin 18.2/CD3 TsAb in TriSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSQVQLVESGGGLVQPGGSLRLSCAASGFTFSNYAMYWVRQAPGKGPEWVSVISGGGGVISYADSVKGRFTISRDNAKNTMYLQMNSLKPEDTAVYYCNAQSAVWWENDYWGQGTQVTVSS

SEQ ID NO: 65 - protein, 578 aa, Artificial
  Exemplary chain 2 sequence of Claudin 18.2/CD3 TsAb in TriSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSQVQLVESGGGLVQPGGSLRLSCAASGFTFSNYAMYWVRQAPGKGPEWVSVISGGGGVISYADSVKGRFTISRDNAKNTMYLQMNSLKPEDTAVYYCNAQSAVWWENDYWGQGTQVTVSS

SEQ ID NO: 66 - protein, 213 aa, Artificial; identical to SEQ ID NO: 45
  Exemplary chain 3 sequence of Claudin 18.2/CD3 TsAb in TriSpecific-V3 format

SEQ ID NO: 67 - protein, 580 aa, Artificial
  Exemplary chain 1 sequence of Claudin 18.2/CD3 BsAb in BiSpecific-V4 format
QVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSSGGGGSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 575 aa, Artificial
  Exemplary chain 1 sequence of Claudin 18.2/CD3 BsAb in BiSpecific-V4 format
QVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 213 aa, Artificial; identical to SEQ ID NO: 45
  Exemplary chain 2 sequence of Claudin 18.2/CD3 BsAb in BiSpecific-V4 format

SEQ ID NO: 70 - protein, 580 aa, Artificial
  Exemplary chain 1 sequence of Claudin 18.2/CD3 TsAb in TriSpecific-V4 format
QVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSSGGGGSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 575 aa, Artificial
  Exemplary chain 1 sequence of Claudin 18.2/CD3 TsAb in TriSpecific-V4 format
QVQLVESGGGLVQAGGSLKLSCTASGDRFSSYAMGWFRQAPGKEREFVAAVSWSGRITDYADSVKGRFTISRDNAKDTVYLQMNNLKPEDTSVYYCYANLLWRDHWGQGTLVTVSSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 583 aa, Artificial
  Exemplary chain 2 sequence of Claudin 18.2/CD3 TsAb in TriSpecific-V4 format
QVQLVESGGGLVQPGGSLRLSCAASGFTFSNYAMYWVRQAPGKGPEWVSVISGGGGVISYADSVKGRFTISRDNAKNTMYLQMNSLKPEDTAVYYCNAQSAVWWENDYWGQGTQVTVSSGGGGSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 73 - protein, 578 aa, Artificial
  Exemplary chain 2 sequence of Claudin 18.2/CD3 TsAb in TriSpecific-V4 format
QVQLVESGGGLVQPGGSLRLSCAASGFTFSNYAMYWVRQAPGKGPEWVSVISGGGGVISYADSVKGRFTISRDNAKNTMYLQMNSLKPEDTAVYYCNAQSAVWWENDYWGQGTQVTVSSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 74 - protein, 213 aa, Artificial; identical to SEQ ID NO: 45
  Exemplary chain 3 sequence of Claudin 18.2/CD3 TsAb in TriSpecific-V4 format

SEQ ID NO: 75 - protein, 344 aa, Artificial
  Exemplary chain 1 sequence of Claudin 18.2/CD3 BsAb in BiSpecific-V1 format
QVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 344 aa, Artificial
  Exemplary chain 1 sequence of Claudin 18.2/CD3 BsAb in BiSpecific-V1 format
QVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 478 aa, Artificial
  Exemplary chain 1 sequence of Claudin 18.2/CD3 TsAb in TriSpecific-V1 format
QVQLVESGGGLVQPGGSLRLSCAASGFTFSNYAMYWVRQAPGKGPEWVSVISGGGGVISYADSVKGRFTISRDNAKNTMYLQMNSLKPEDTAVYYCNAQSAVWWENDYWGQGTQVTVSSGGGGSGGGGSGGGGSQVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 473 aa, Artificial
  Exemplary chain 1 sequence of Claudin 18.2/CD3 TsAb in TriSpecific-V1 format
QVQLVESGGGLVQPGGSLRLSCAASGFTFSNYAMYWVRQAPGKGPEWVSVISGGGGVISYADSVKGRFTISRDNAKNTMYLQMNSLKPEDTAVYYCNAQSAVWWENDYWGQGTQVTVSSGGGGSGGGGSQVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 344 aa, Artificial; identical to SEQ ID NO: 75
  Exemplary chain 1 sequence of Claudin 18.2/CD3 BsAb in BiSpecific-V2 format

SEQ ID NO: 80 - protein, 344 aa, Artificial; identical to SEQ ID NO: 76
  Exemplary chain 1 sequence of Claudin 18.2/CD3 BsAb in BiSpecific-V2 format

SEQ ID NO: 81 - protein, 478 aa, Artificial; identical to SEQ ID NO: 77
  Exemplary chain 1 sequence of Claudin 18.2/CD3 TsAb in TriSpecific-V2 format

SEQ ID NO: 82 - protein, 473 aa, Artificial; identical to SEQ ID NO: 78
  Exemplary chain 1 sequence of Claudin 18.2/CD3 TsAb in TriSpecific-V2 format

SEQ ID NO: 83 - protein, 576 aa, Artificial
  Exemplary chain 1 sequence of Claudin 18.2/CD3 BsAb in BiSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSS

SEQ ID NO: 84 - protein, 571 aa, Artificial
  Exemplary chain 1 sequence of Claudin 18.2/CD3 BsAb in BiSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSS

SEQ ID NO: 85 - protein, 576 aa, Artificial
  Exemplary chain 1 sequence of Claudin 18.2/CD3 TsAb in TrSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSS

SEQ ID NO: 86 - protein, 571 aa, Artificial
  Exemplary chain 1 sequence of Claudin 18.2/CD3 TsAb in TrSpecific-V3 format
EVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSS

SEQ ID NO: 87 - protein, 576 aa, Artificial
  Exemplary chain 1 sequence of Claudin 18.2/CD3 BsAb in BiSpecific-V4 format
QVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSSGGGGSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 571 aa, Artificial
  Exemplary chain 1 sequence of Claudin 18.2/CD3 BsAb in BiSpecific-V4 format
QVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 576 aa, Artificial
  Exemplary chain 1 sequence of Claudin 18.2/CD3 TsAb in TriSpecific-V4 format
QVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSSGGGGSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 571 aa, Artificial
  Exemplary chain 1 sequence of Claudin 18.2/CD3 TsAb in TriSpecific-V4 format
QVQLVESGGGLVQAGGSLRLSCAASGLTFSLYDMGWFRQAPGKEREWVATITSSGNTGYAESVKGRFTVSRDNGKKTGYLQTNNLKPEDTALYYCYVPFLGRGYGTLVTVSSGGGGSGGGGSEVQLQESGAELARPGASVKMSCKASGYTFTSYTMHWVKQRPGEGLEWIGYINPSSGYTKYNQKFKDKATLTADKSSSTAYMELSSLTSEDSAVYYCARWQDYDVYFDYWGEGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 116 aa, Artificial
  Humanized Claudin 18.2 VHH sequence
EVQLVESGGGLVQPGGSLRLSCTASGDRFSSYAMGWFRQAPGKEREYVAAVSWSGRITDYADSVKGRFTISRDNSKNTVYLQMNSLRAEDTAVYYCYANLLWRDHWGQGTLVTVSS

SEQ ID NO: 92 - protein, 116 aa, Artificial
  Humanized Claudin 18.2 VHH sequence
EVQLVESGGGLVQPGGSLRLSCTASGDRFSSYAMGWFRQAPGKEREFVAAVSWSGRITDYADSVKGRFTISRDNSKNTVYLQMNSLKAEDTSVYYCYANLLWRDHWGQGTLVTVSS

SEQ ID NO: 93 - protein, 112 aa, Artificial
  Humanized Claudin 18.2 VHH sequence
EVQLVESGGGLVQPGGSLRLSCAASGLTFSLYDMGWVRQAPGKEREWVATITSSGNTGYAESVKGRFTVSRDNAKNSGYLQTNSLRAEDTAVYYCYVPFLGRGRGTLVTVSS

SEQ ID NO: 94 - protein, 112 aa, Artificial
  Humanized Claudin 18.2 VHH sequence
EVQLVESGGGLVQPGGSLRLSCAASGLTFSLYDMGWFRQAPGKEREWVATITSSGNTGYAESVKGRFTVSRDNAKNSGYLQTNSLRAEDTAVYYCYVPFLGRGYGTLVTVSS

SEQ ID NO: 95 - protein, 119 aa, Artificial
  CD3 VH sequence
QVQLQESGAEVAKPGASVKMSCKASGYTFTSYTMHWVKQRPGEGLEWIGYINPSSGYTKYNQKFKDRATLTADKSSSTAYMELSSLTSEDTAVYYCARWQDYDVYFDYWGEGTTLTVSS

SEQ ID NO: 96 - protein, 106 aa, Artificial
  CD3 VL sequence
QIVLTQSPASLSASPGDRVTMTCRASSSVSYIHWFQQKPGSSPKPWIYATSNLASGVPSRFSGSGSGTSYTLTISRVEPEDFATYYCQQWTSNPPTFGGGTKLEIK

(19) United States
(12) Patent Application Publication
(10) Pub. No.: US 2024/0109963 A1
(43) Pub. Date: Apr. 4, 2024

(54) MULTISPECIFIC ANTIBODIES AND USES THEREOF

(71) Applicant: Ab Therapeutics, Inc.

(72) Inventors: Amit K. Chaudhary, San Lorenzo, CA (US); Yue Liu, Foster City, CA (US)

(21) Appl. No.: 18/036,250

(22) PCT Filed: Nov. 16, 2021
(86) PCT No.: PCT/US2021/059528

§ 371 Date: May 10, 2023

Related U.S. Application Data
(60) Provisional application No. 63/114,495, filed on Nov. 16, 2020.

(51) Int. Cl. C07K 16/28; A61K 47/68; A61P 35/00

(52) U.S. Cl. CPC C07K 16/2809; A61K 47/6849; A61P 35/00; C07K 16/28; C07K 2317/31; C07K 2317/569; C07K 2317/622

(57) ABSTRACT

This disclosure relates to multispecific antibodies (e.g., bispecific antibodies) or antigenbinding fragments thereof. In one aspect, the multispecific antibodies or antigen-binding fragments thereof binds to CD3, Claudin 18.2, or a combination thereof.

### CLAIM OF PRIORITY

This application claims the benefit of U.S. Provisional Application No. 63/114,495, filed on Nov. 16, 2020. The entire contents of the foregoing application are incorporated herein by reference.

### TECHNICAL FIELD

This disclosure relates to multispecific antibodies or antigen-binding fragments thereof.

### BACKGROUND

A multispecific antibody is an artificial protein that can simultaneously bind to two or more different epitopes. This opens up a wide range of applications, including redirecting T cells to tumor cells, blocking two different signaling pathways simultaneously, dual targeting of different disease mediators, and delivering payloads to targeted sites. The approval of catumaxomab (anti-EpCAM and anti-CD3) and blinatumomab (anti-CD19 and anti-CD3) has become a major milestone in the development of multispecific antibodies.

As multispecific antibodies have various applications, there is a need to continue to develop various therapeutics based on multispecific antibodies.

### SUMMARY

This disclosure relates to antibodies or antigen-binding fragments, wherein the antibodies or antigen-binding fragments specifically bind to CD3, and/or Claudin 18.2, or a combination thereof. In some embodiments, the disclosure is related to development of an “imbalanced” Claudin 18.2/CD3 bispecific antibody with “balanced” safety, efficacy and developability via computer aided design.

In one aspect, the disclosure is related to an antibody or antigen-binding fragment thereof that binds to Claudin 18.2, comprising: a heavy-chain antibody variable domain (VHH) comprising complementarity determining regions (CDRs) 1, 2, and 3.

In some embodiments, the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence.

In some embodiments, the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively; and
(4) the selected VHH CDRs 1, 2, 3 amino acid sequences are set forth in SEQ ID NOs: 10, 11, and 12, respectively.

In some embodiments, the VHH comprises CDRs 1, 2, 3 with the amino acid sequences set forth in SEQ ID NOs: 1, 2, and 3, respectively.

In some embodiments, the VHH comprises CDRs 1, 2, 3 with the amino acid sequences set forth in SEQ ID NOs: 4, 5, and 6, respectively.

In some embodiments, the VHH comprises CDRs 1, 2, 3 with the amino acid sequences set forth in SEQ ID NOs: 7, 8, and 9, respectively.

In some embodiments, the VHH comprises CDRs 1, 2, 3 with the amino acid sequences set forth in SEQ ID NOs: 10, 11, and 12, respectively.

In one aspect, the disclosure is related to an antibody or antigen-binding fragment thereof that binds to Claudin 18.2 comprising a heavy-chain antibody variable domain (VHH) comprising an amino acid sequence that is at least 80% identical to a selected VHH sequence.

In some embodiments, the selected VHH sequence is selected from the group consisting of SEQ ID NOs: 19-22 and 91-94. In some embodiments, the VHH comprises the sequence of SEQ ID NO: 19, 20, 21, or 22. In some embodiments, the VHH comprises the sequence of SEQ ID NO: 21. In some embodiments, the VHH comprises the sequence of SEQ ID NO: 91 or 92. In some embodiments, the VHH comprises the sequence of SEQ ID NO: 93 or 94.

In some embodiments, the antibody or antigen-binding fragment specifically binds to Claudin 18.2.

In some embodiments, the antibody or antigen-binding fragment is a humanized antibody or antigen-binding fragment thereof.

In one aspect, the disclosure is related to an antibody or antigen-binding fragment thereof comprising the VHH CDRs 1, 2, 3, of the antibody or antigen-binding fragment thereof as described herein.

In some embodiments, the antibody or antigen-binding fragment comprises a human IgG Fc.

In some embodiments, the antibody or antigen-binding fragment comprises two or more heavy-chain antibody variable domains.

In one aspect, the disclosure is related to an antibody or antigen-binding fragment thereof that cross-competes with the antibody or antigen-binding fragment thereof described herein.

In one aspect, the disclosure is related to an antibody or antigen-binding fragment thereof that binds to CD3 (cluster of differentiation 3) comprising: a heavy chain variable region (VH) comprising complementarity determining regions (CDRs) 1, 2, and 3, and a light chain variable region (VL) comprising CDRs 1, 2, and 3.

In some embodiments, the VH CDR1 region comprises an amino acid sequence that is at least 80% identical to SEQ ID NO: 13, the VH CDR2 region comprises an amino acid sequence that is at least 80% identical to SEQ ID NO: 14, and the VH CDR3 region comprises an amino acid sequence that is at least 80% identical to SEQ ID NO: 15.

In some embodiments, the VL CDR1 region comprises an amino acid sequence that is at least 80% identical to SEQ ID NO: 16, the VL CDR2 region comprises an amino acid sequence that is at least 80% identical to SEQ ID NO: 17, and the VL CDR3 region comprises an amino acid sequence that is at least 80% identical to SEQ ID NO: 18.

In some embodiments, the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17, and 18, respectively.

In one aspect, the disclosure is related to an antibody or antigen-binding fragment thereof that binds to CD3 comprising a heavy chain variable region (VH) comprising an amino acid sequence that is at least 90% identical to SEQ ID NO: 23 or 95, and a light chain variable region (VL) comprising an amino acid sequence that is at least 90% identical to SEQ ID NO: 24 or 96. In some embodiments, the VH comprises the sequence of SEQ ID NO: 23 and the VL comprises the sequence of SEQ ID NO: 24. In some embodiments, the VH comprises the sequence of SEQ ID NO: 95 and the VL comprises the sequence of SEQ ID NO: 96.

In some embodiments, the antibody or antigen-binding fragment specifically binds to human CD3.

In some embodiments, the antibody or antigen-binding fragment is a single-chain variable fragment (scFV).

In one aspect, the disclosure is related to an antibody or antigen-binding fragment thereof comprising the VH CDRs 1, 2, 3, and VL CDRs 1, 2, 3, of the antibody or antigen-binding fragment thereof as described herein.

In one aspect, the disclosure is related to an antibody or antigen-binding fragment thereof that cross-competes with the antibody or antigen-binding fragment thereof as described herein.

In some embodiments, the antibody is a bispecific antibody or a multispecific antibody.

In one aspect, the disclosure is related to a multi-specific antibody or antigen-binding fragment thereof, comprising a first antigen-binding site that specifically binds to CD3, and a second antigen-binding site that specifically binds to Claudin 18.2.

In some embodiments, the first antigen-binding site comprises a heavy chain variable region (VH) and a light chain variable region (VL). In some embodiments, the VH and the VL associate with each other and specifically bind to CD3.

In some embodiments, the heavy chain variable region (VH) comprising complementarity determining regions (CDRs) 1, 2, and 3. In some embodiments, the VH CDR1 region comprises an amino acid sequence that is at least 80% identical to SEQ ID NO: 13, the VH CDR2 region comprises an amino acid sequence that is at least 80% identical to SEQ ID NO: 14, and the VH CDR3 region comprises an amino acid sequence that is at least 80% identical to SEQ ID NO: 15.

In some embodiments, the light chain variable region (VL) comprising CDRs 1, 2, and 3. In some embodiments, the VL CDR1 region comprises an amino acid sequence that is at least 80% identical to SEQ ID NO: 16, the VL CDR2 region comprises an amino acid sequence that is at least 80% identical to SEQ ID NO: 17, and the VL CDR3 region comprises an amino acid sequence that is at least 80% identical to SEQ ID NO: 18.

In some embodiments, the heavy chain variable region (VH) comprises an amino acid sequence that is at least 90% identical to SEQ ID NO: 23 or 95, and a light chain variable region (VL) comprising an amino acid sequence that is at least 90% identical to SEQ ID NO: 24 or 96.

In some embodiments, the second antigen-binding site specifically binds to Claudin 18.2, and the second antigen-binding site comprises a first heavy-chain antibody variable domain (VHH1) comprising complementarity determining regions (CDRs) 1, 2, and 3. In some embodiments, the VHH1 CDR1 region comprises an amino acid sequence that is at least 80% identical to a selected VHH1 CDR1 amino acid sequence, the VHH1 CDR2 region comprises an amino acid sequence that is at least 80% identical to a selected VHH1 CDR2 amino acid sequence, and the VHH1 CDR3 region comprises an amino acid sequence that is at least 80% identical to a selected VHH1 CDR3 amino acid sequence. In some embodiments, the selected VHH1 CDRs 1, 2, and 3 amino acid sequences are one of the following:
(1) the selected VHH1 CDRs 1, 2, 3 amino acid sequences are set forth in SEQ ID NOs: 1, 2, and 3, respectively;
(2) the selected VHH1 CDRs 1, 2, 3 amino acid sequences are set forth in SEQ ID NOs: 4, 5, and 6, respectively;
(3) the selected VHH1 CDRs 1, 2, 3 amino acid sequences are set forth in SEQ ID NOs: 7, 8, and 9, respectively; and
(4) the selected VHH1 CDRs 1, 2, 3 amino acid sequences are set forth in SEQ ID NOs: 10, 11, and 12, respectively.

In some embodiments, the first heavy-chain antibody variable domain (VHH1) comprises an amino acid sequence that is at least 80% identical to a selected VHH1 sequence. In some embodiments, the selected VHH1 sequence is selected from the group consisting of SEQ ID NOs: 19-22 and 91-94.

In some embodiments, the multi-specific antibody or antigen-binding fragment thereof as described herein further comprises a third antigen-binding site that specifically binds to Claudin 18.2.

In some embodiments, the third antigen-binding site specifically binds to Claudin 18.2, and the third antigen-binding site comprises a second heavy-chain antibody variable domain (VHH2) comprising complementarity determining regions (CDRs) 1, 2, and 3. In some embodiments, the VHH2 CDR1 region comprises an amino acid sequence that is at least 80% identical to a selected VHH2 CDR1 amino acid sequence, the VHH2 CDR2 region comprises an amino acid sequence that is at least 80% identical to a selected VHH2 CDR2 amino acid sequence, and the VHH2 CDR3 region comprises an amino acid sequence that is at least 80% identical to a selected VHH2 CDR3 amino acid sequence. In some embodiments, the selected VHH2 CDRs 1, 2, and 3 amino acid sequences are one of the following:
(1) the selected VHH2 CDRs 1, 2, 3 amino acid sequences are set forth in SEQ ID NOs: 1, 2, and 3, respectively;
(2) the selected VHH2 CDRs 1, 2, 3 amino acid sequences are set forth in SEQ ID NOs: 4, 5, and 6, respectively;
(3) the selected VHH2 CDRs 1, 2, 3 amino acid sequences are set forth in SEQ ID NOs: 7, 8, and 9, respectively; and
(4) the selected VHH2 CDRs 1, 2, 3 amino acid sequences are set forth in SEQ ID NOs: 10, 11, and 12, respectively.

In some embodiments, the second heavy-chain antibody variable domain (VHH2) comprises an amino acid sequence that is at least 80% identical to a selected VHH2 sequence. In some embodiments, the selected VHH2 sequence is selected from the group consisting of SEQ ID NOs: 19-22 and 91-94.

In some embodiments, the VH and the VL are linked by a linker peptide sequence to form an scFv.

In some embodiments, the linker peptide sequence comprises a sequence that is at least 80% identical to any one of SEQ ID NOs: 35-40.

In one aspect, the disclosure is related to a polypeptide complex, comprising (a) a first polypeptide comprising from N-terminus to C-terminus: a first heavy-chain antibody variable domain (VHH), a first hinge region, a first Fc region; (b) a second polypeptide comprising from N-terminus to C-terminus: a heavy chain variable region (VH), a second hinge region, and a second Fc region; and (c) a third polypeptide comprising a light chain variable region (VL).

In some embodiments, the first VHH specifically binds to Claudin 18.2. In some embodiments, the VH and the VL associate with each other and specifically bind to CD3.

In some embodiments, the first hinge region and/or the second hinge region comprise a sequence that is at least 80% identical to any one of SEQ ID NOs: 25-29.

In some embodiments, the first Fc region and/or the second Fc region comprise a sequence that is at least 80% identical to SEQ ID NO: 30 or 31.

In some embodiments, the first polypeptide comprises a sequence that is at least 80% identical to SEQ ID NO: 41,42, 75, or 76. In some embodiments, the second polypeptide comprises a sequence that is at least 80% identical to SEQ ID NO: 43 or 44. In some embodiments, the third polypeptide comprises a sequence that is at least 80% identical to SEQ ID NO: 45.

In some embodiments, the first polypeptide further comprises a second VHH that specifically binds to Claudin 18.2. In some embodiments, the second VHH is linked to the N-terminus of the first VHH via a linker peptide sequence.

In some embodiments, the linker peptide sequence comprises a sequence that is at least 80% identical to any one of SEQ ID NOs: 35-40.

In some embodiments, the first polypeptide comprises a sequence that is at least 80% identical to SEQ ID NO: 46,47, 77, or 78. In some embodiments, the second polypeptide comprises a sequence that is at least 80% identical to SEQ ID NO: 48 or 49. In some embodiments, the third polypeptide comprises a sequence that is at least 80% identical to SEQ ID NO: 50.

In one aspect, the disclosure is related to a polypeptide complex, comprising (a) a first polypeptide comprising from N-terminus to C-terminus: a first heavy-chain antibody variable domain (VHH), a first hinge region, a first Fc region; and (b) a second polypeptide comprising from N-terminus to C-terminus: a single-chain variable fragment (scFv), a second hinge region, and a second Fc region. In some embodiments, the first VHH specifically binds to Claudin 18.2.

In some embodiments, the scFv comprises a heavy chain variable region (VH), a first linker peptide sequence, and a light chain variable region (VL). In some embodiments, the VH and the VL associate with each other and specifically bind to CD3.

In some embodiments, the first linker peptide sequence comprises a sequence that is at least 80% identical to any one of SEQ ID NOs: 35-40.

In some embodiments, the first hinge region and/or the second hinge region comprise a sequence that is at least 80% identical to any one of SEQ ID NOs: 25-29.

In some embodiments, the first Fc region and/or the second Fc region comprise a sequence that is at least 80% identical to SEQ ID NO: 30 or 31.

In some embodiments, the first polypeptide comprises a sequence that is at least 80% identical to SEQ ID NO: 51,52, 79, or 80. In some embodiments, the second polypeptide comprises a sequence that is at least 80% identical to SEQ ID NO: 53 or 54.

In some embodiments, the first polypeptide further comprises a second VHH that specifically binds to Claudin 18.2. In some embodiments, the second VHH is linked to the N-terminus of the first VHH via a second linker peptide sequence.

In some embodiments, the second linker peptide sequence comprises a sequence that is at least 80% identical to any one of SEQ ID NOs: 35-40.

In some embodiments, the first polypeptide comprises a sequence that is at least 80% identical to SEQ ID NO: 55,56, 81, or 82. In some embodiments, the second polypeptide comprises a sequence that is at least 80% identical to SEQ ID NO: 57 or 58.

In one aspect, the disclosure is related to a polypeptide complex, comprising (a) a first polypeptide comprising from N-terminus to C-terminus: a first heavy chain variable region (VH1), a first hinge region, a first Fc region; a first linker peptide sequence, and a first heavy-chain antibody variable domain (VHH1); (b) a second polypeptide comprising a first light chain variable region (VL1); (c) a third polypeptide comprising from N-terminus to C-terminus: a second heavy chain variable region (VH2), a second hinge region, a second Fc region, a second linker peptide sequence, and a second heavy-chain antibody variable domain (VHH2); and (d) a fourth polypeptide comprising a second light chain variable region (VL2). In some embodiments, the VHH1 and/or the VHH2 specifically bind to Claudin 18.2. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80% identical to any one of SEQ ID NOs: 35-40.

In some embodiments, the first hinge region and/or the second hinge regions comprise a sequence that is at least 80% identical to any one of SEQ ID NOs: 25-29.

In some embodiments, sequences of the VHH1 and the VHH2 are identical. In some embodiments, the first Fc region and/or the second Fc region comprise a sequence that is at least 80% identical to SEQ ID NO: 32. In some embodiments, the first polypeptide comprises a sequence that is at least 80% identical to SEQ ID NO: 59,60, 83, or 84. In some embodiments, the second polypeptide comprises a sequence that is at least 80% identical to SEQ ID NO: 61. In some embodiments, the third polypeptide comprises a sequence that is at least 80% identical to SEQ ID NO: 59,60, 83, or 84. In some embodiments, the fourth polypeptide comprises a sequence that is at least 80% identical to SEQ ID NO: 61.

In some embodiments, sequences of the VHH1 and the VHH2 are different. In some embodiments, the first Fc region and/or the second Fc region comprise a sequence that is at least 80% identical to SEQ ID NO: 30 or 31. In some embodiments, the first polypeptide comprises a sequence that is at least 80% identical to SEQ ID NO: 62,63, 85, or 86. In some embodiments, the second polypeptide comprises a sequence that is at least 80% identical to SEQ ID NO: 66. In some embodiments, the third polypeptide comprises a sequence that is at least 80% identical to SEQ ID NO: 64 or 65. In some embodiments, the fourth polypeptide comprises a sequence that is at least 80% identical to SEQ ID NO: 66.

In one aspect, the disclosure is related to a polypeptide complex, comprising (a) a first polypeptide comprising from N-terminus to C-terminus: a first heavy-chain antibody variable domain (VHH1), a first linker peptide sequence, a first heavy chain variable region (VH1), a first hinge region, and a first Fc region; (b) a second polypeptide comprising a first light chain variable region (VL1); (c) a third polypeptide comprising from N-terminus to C-terminus: a second heavy-chain antibody variable domain (VHH2), a second linker peptide sequence, a second heavy chain variable region (VH2), a second hinge region, and a second Fc region; and (d) a fourth polypeptide comprising a second light chain variable region (VL2). In some embodiments, the VHH1 and the VHH2 specifically bind to Claudin 18.2. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80% identical to any one of SEQ ID NOs: 35-40.

In some embodiments, the first hinge region and/or the second hinge regions comprise a sequence that is at least 80% identical to any one of SEQ ID NOs: 25-29.

In some embodiments, sequences of the VHH1 and the VHH2 are identical. In some embodiments, the first Fc region and/or the second Fc region comprise a sequence that is at least 80% identical to SEQ ID NO: 32. In some embodiments, the first polypeptide comprises a sequence that is at least 80% identical to SEQ ID NO: 67,68, 87, or 88. In some embodiments, the second polypeptide comprises a sequence that is at least 80% identical to SEQ ID NO: 69. In some embodiments, the third polypeptide comprises a sequence that is at least 80% identical to SEQ ID NO: 67,68, 87, or 88. In some embodiments, the fourth polypeptide comprises a sequence that is at least 80% identical to SEQ ID NO: 69.

In some embodiments, sequences of the VHH1 and the VHH2 are different. In some embodiments, the first Fc region and/or the second Fc region comprise a sequence that is at least 80% identical to SEQ ID NO: 30 or 31. In some embodiments, the first polypeptide comprises a sequence that is at least 80% identical to SEQ ID NO: 70,71, 89, or 90. In some embodiments, the second polypeptide comprises a sequence that is at least 80% identical to SEQ ID NO: 74. In some embodiments, the third polypeptide comprises a sequence that is at least 80% identical to SEQ ID NO: 72 or 73. In some embodiments, the fourth polypeptide comprises a sequence that is at least 80% identical to SEQ ID NO: 74.

In one aspect, the disclosure is related to a nucleic acid comprising a polynucleotide encoding the antibody or antigen-binding fragment thereof as described herein, the multi-specific antibody or antigen-binding fragment thereof as described herein, or the polypeptide complex as described herein.

In some embodiments, the nucleic acid is a DNA (e.g., cDNA) or RNA (e.g., mRNA).

In one aspect, the disclosure is related to a vector comprising one or more of the nucleic acids as described herein.

In one aspect, the disclosure is related to a cell comprising the vector as described herein.

In some embodiments, the cell is a CHO cell.

In one aspect, the disclosure is related to a cell comprising one or more of the nucleic acids as described herein.

In one aspect, the disclosure is related to a method of producing an antibody or an antigen-binding fragment thereof, the method comprising (a) culturing the cell as described herein under conditions sufficient for the cell to produce the antibody or the antigen-binding fragment; and (b) collecting the antibody or the antigen-binding fragment produced by the cell.

In one aspect, the disclosure is related to an antibody-drug conjugate comprising the antibody or antigen-binding fragment thereof as described herein, or the multi-specific antibody or antigen-binding fragment thereof as described herein, covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof as described herein, the multi-specific antibody or antigen-binding fragment thereof as described herein, the polypeptide complex as described herein, or the antibody-drug conjugate as described herein, to the subject.

In some embodiments, the subject has a cancer expressing Claudin 18.2.

In some embodiments, the cancer is a solid tumor. In some embodiments, the cancer is gastric or pancreatic adenocarcinoma.

In one aspect, the disclosure is related to a method of decreasing the rate of tumor growth, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure is related to a method of killing a tumor cell, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure is related to a method of killing a tumor cell, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure is related to a pharmaceutical composition comprising the antibody or antigen-binding fragment thereof as described herein, the multi-specific antibody or antigen-binding fragment thereof as described herein, or the polypeptide complex as described herein, and a pharmaceutically acceptable carrier.

In one aspect, the disclosure is related to a pharmaceutical composition comprising the antibody-drug conjugate as described herein, and a pharmaceutically acceptable carrier.

In one aspect, the disclosure provides a multi-specific antibody or antigen-binding fragment thereof, comprising the heavy-chain antibody variable domain (VHH) as described herein, or the heavy chain variable region (VH) and the light chain variable region (VL) as described herein.

In one aspect, the disclosure provides a bispecific antibody or antigen-binding fragment thereof, comprising the heavy-chain antibody variable domain (VHH) as described herein, or the heavy chain variable region (VH) and the light chain variable region (VL) as described herein.

In one aspect, the disclosure provides a multi-specific antibody or antigen-binding fragment thereof that specifically binds to Claudin 18.2 and/or CD3.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)2, and Fv fragments, ScFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to CD3 may be referred to as a CD3-specific antibody or an anti-CD3 antibody.

As used herein, the term “bispecific antibody” refers to an antibody that binds to two different epitopes. The epitopes can be on the same antigen or on different antigens.

As used herein, the term “tripecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “VHH” refers to the variable domain of a heavy chain antibody. In some embodiments, the VHH is a humanized VHH.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1B show percentage of Claudin 18.1-expressing CHO cells (CHO_18.2), Claudin 18.1-expressing CHO cells (CHO_18.1), and normal CHO cells (CHO) that are bound with the Claudin 18.2-targeting antibody candidates. Cells bound with the antibody candidates are PE positive.

FIG. 2 shows binding curves of Claudin 18.2-expressing CHO cells (CHO+ Claudin 18.2) or control CHO cells (CHO) to unpurified cell culture supernatants containing Claudin 18.2-1A11/CD3, Claudin 18.2-1B2/CD3, Claudin 18.2-3C2/CD3, and Claudin 18.2-4G1/CD3 BsAbs. Cells bound with the BsAbs are FITC-positive.

FIG. 3 shows induction of T cell activation by Claudin 18.2-1A11/CD3 in the presence of Claudin 18.2-overexpressing MIA PaCa-2 pancreatic cancer cells and human PBMCs. T cell activation was analyzed by co-staining CD2 T-cell specific marker and CD69 T-cell activation marker by flow cytometry. Anti-his tag antibody is an isotype control.

FIG. 4 shows induction of tumor cell killing (TCK) by Claudin 18.2-1A11/CD3 in the presence of Claudin 18.2-overexpressing MIA PaCa-2 pancreatic cancer cell and human PBMCs. The TCK was analyzed by crystal violet staining of live adherent cancer cells.

FIG. 5 shows induction of complement dependent cytotoxicity (CDC) by Claudin 18.2-1A11/CD3 in the presence of 10% pooled human complement serum against Claudin 18.2-overexpressing CHO cells or wild-type CHO cells. The CDC was analyzed by LDH cytotoxicity assays.

FIG. 6A shows a schematic structure of an exemplary Claudin 18.2/CD3 bispecific antibody with the BiSpecific-V1 format.

FIG. 6B shows a schematic structure of an exemplary Claudin 18.2/CD3 trispecific antibody with the TriSpecific-V1 format.

FIG. 7A shows a schematic structure of an exemplary Claudin 18.2/CD3 bispecific antibody with the BiSpecific-V2 format.

FIG. 7B shows a schematic structure of an exemplary Claudin 18.2/CD3 trispecific antibody with the TriSpecific-V2 format.

FIG. 8A shows a schematic structure of an exemplary Claudin 18.2/CD3 bispecific antibody with the BiSpecific-V3 format.

FIG. 8B shows a schematic structure of an exemplary Claudin 18.2/CD3 trispecific antibody with the TriSpecific-V3 format.

FIG. 9A shows a schematic structure of an exemplary Claudin 18.2/CD3 bispecific antibody with the BiSpecific-V4 format.

FIG. 9B shows a schematic structure of an exemplary Claudin 18.2/CD3 trispecific antibody with the TriSpecific-V4 format.

FIG. 10 is a schematic diagram showing how a bispecific antibody that binds to CD3 and a cancer antigen (e.g., cancer specific antigen) can recognize and kill a tumor cell.

FIG. 11A shows binding curves of Claudin 18.2-expressing CHO cells (CHO+ Claudin 18.2) to Claudin 18.2-1A11/CD3 BsAb, Claudin 18.2-1B2/CD3 BsAb, and AMG910 analog. Cells bound with the antibodies are FITC-positive.

FIG. 11B shows binding curves of Jurkat cells to Claudin 18.2-1A11/CD3 BsAb, Claudin 18.2-1B2/CD3 BsAb, and AMG910 analog. Cells bound with the antibodies are FITC-positive.

FIG. 12A shows binding curves of Claudin 18.2-expressing CHO cells (CHO+ Claudin 18.2) to Clnd18.2-1A11/3C2FC/CD3 (up) TsAb, Clnd18.2-1A11/3C2/CD3-H/L(up) TsAb, and AMG910 analog. Cells bound with the antibodies are FITC-positive.

FIG. 12B shows binding curves of Jurkat cells to Clnd18.2-1A11/3C2FC/CD3(up) TsAb, Clnd18.2-1A11/3C2/CD3-H/L(up) TsAb, and AMG910 analog. Cells bound with the antibodies are FITC-positive.

FIG. 13 shows induction of T cell activation by Claudin 18.2-1B2/CD3 BsAb, Claudin 18.2-1A11/3C2FC/CD3 TsAb, Claudin 18.2-1B2/3C2FC/CD3 TsAb, and AMG910 analog in the presence of Claudin 18.2-overexpressing MIA PaCa-2 pancreatic cancer cells and human PBMCs. T cell activation was analyzed by staining CD69 T-cell activation marker by flow cytometry. Anti-his tag antibody is an isotype control.

FIG. 14A shows induction of tumor cell killing (TCK) by Clnd18.2/CD3-V3-3C2/1A11(up) TsAb, Clnd18.2-1A11/3C2/CD3-H/L(up) TsAb, and AMG910 analog in the presence of Claudin 18.2-overexpressing MIA PaCa-2 pancreatic cancer cell and human PBMCs. The TCK was analyzed by crystal violet staining of live adherent cancer cells.

FIG. 14B shows T cell depletion results of Clnd18.2/CD3-V3-3C2/1A11(up) TsAb, Clnd18.2-1A11/3C2/CD3-H/L(up) TsAb, and AMG910 analog in the presence of Claudin 18.2-overexpressing MIA PaCa-2 pancreatic cancer cell and human PBMCs.

FIGS. 15A-15F show the percentage of CD25+ cells gated on CD2+ cells using PBMCs from 6 donors. The percentage indicates T cell activation of PBMCs in the absence or presence of Claudin18.2-overexpressing MIA PaCa cells, after treatment with Claudin 18.2-1B2/CD3 BsAb. AMG910 analog and an anti-His antibody were used as negative controls.

FIGS. 16A-16F show relative CD25 MFI gated on CD2+ cells using PBMCs from 6 donors. The relative CD25 MFI indicates T cell activation of PBMCs in the absence or presence of Claudin18.2-overexpressing MIA PaCa cells, after treatment with Claudin 18.2-1B2/CD3 BsAb. AMG910 analog and an anti-His antibody were used as negative controls.

FIGS. 17A-17F show cancer cell killing curves using PBMCs from 6 donors in the absence or presence of Claudin18.2-overexpressing MIA PaCa cells. The cell mixture was treated with Claudin 18.2-1B2/CD3 BsAb. AMG910 analog and an anti-His antibody were used as negative controls.

FIGS. 18A-18F show IFN-7 release levels using PBMCs from 6 donors in the absence or presence of Claudin18.2-overexpressing MIA PaCa cells. The cell mixture was treated with Claudin 18.2-1B2/CD3 BsAb. AMG910 analog and an anti-His antibody were used as negative controls.

FIG. 19 lists CDR sequences of the VHHs from the Claudin 18.2 antibodies described in the disclosure.

FIG. 20 lists CDR sequences of the heavy chain variable region (VH) of the CD3 antibodies described in the disclosure.

FIG. 21 lists CDR sequences of the light chain variable region (VL) of the CD3 antibodies described in the disclosure.

FIG. 22 lists amino acid sequences of VHHs that specifically bind to Claudin 18.2 as described in the disclosure.

FIG. 23 lists amino acid sequences of VH and VL that specifically bind to CD3 as described in the disclosure.

FIG. 24 lists sequences discussed in the disclosure.

### DETAILED DESCRIPTION

Human cancer is a chronic and pro-inflammatory disease that rapidly advances to an uncontrollable stage. The solid tumors are heterogeneous in nature containing cells from the immune system. Tumor-infiltrating lymphocytes (TTLs), T cells, B cells, and natural killer (NK) cells can affect tumor microenvironment by secreting inflammatory mediators, which either initiate or inhibit tumor growth. With the discovery of the underlying cellular mechanisms of cancer progression, multiple therapeutic agents have been developed. These therapeutic agents can exhibit their anti-cancer activities by controlling specific proteins in specific signaling pathways that are aberrantly expressed in tumor cells or tumor microenvironment. However, these therapeutic agents often fail to control advanced tumor because of low stability, poor solubility, drug resistance, and/or less accessibility into the cancer tissues/cells due to non-specific uptake by healthy tissues/cells.

While surgery, radiation, and chemotherapy remain to be the first-line treatment options, harnessing the power of patients' own immune system is becoming a promising alternative for treating cancers. Over the past few decades, exciting technologies and platforms have been developed for the design and production of therapeutic antibodies and immune cell-based immunotherapy. Multiple FDA-approved T cell engaging bispecific antibodies (BsAbs) have been developed for treating cancer patients. These BsAbs have been developed targeting two different epitopes or antigens simultaneously on the same cell or two different cells. In general, antigen-induced cytotoxic T-cell immunity is dependent on cancer cell antigen presentation and its recognition by TCR. By binding to both cancer specific antigen and TCR, BsAbs can redirect T cells towards cancer cells, and induce T-cell-mediated cell killing.

Tight junctions are specialized intercellular membranes in epithelial and endothelial cellular sheets. They control paracellular permeability by acting as a barrier for solute diffusion and can recruit various cytoskeletal and signaling molecules at their cytoplasmic surface. Paracellular ion permeability at tight junctions is largely determined by Claudins, which are a family of transmembrane proteins and one of the crucial components of tight junctions. Claudins are a multigene protein family comprising 27 members. It has been shown that aberration in Claudins expression disrupted tight junction functions and initiated tumor-promoting event in various cancers, including breast, biliary tract, gastric, hepatocellular carcinoma, renal cell carcinoma, pancreatic, non-small cell lung cancer, and mesothelioma. Claudin 18.2 belongs to the family of Claudins with four membrane-spanning domains involved in the formation of tight junctions. Human Claudin 18 has two alternative first exons, giving rise to Claudin 18.1 and Claudin 18.2 isoforms. Transcription profiling of a restricted set of tissues showed that these isoforms have different lineage commitments, with Claudin 18.1 being predominantly expressed in lung tissue. However, Claudin 18.2 is highly expressed in a significant proportion of gastric and pancreatic adenocarcinomas, while normal tissue expression is limited to the epithelium of the stomach. The pattern of Claudin 18.2 expression makes it a potential drug candidate for the treatment of gastric and pancreatic adenocarcinoma. In this disclosure, Claudin 18.2-targeting BsAbs were generated, with one arm targeting cancer cells and other arm targeting T cells through CD3, to treat gastric and pancreatic cancers. By binding to both a cancer antigen (e.g., Claudin 18.2) and CD3, the multispecific antibodies described herein can redirect T cells to recognize tumor cells and induce T cell-mediated cell killing with negligible effect to T-cells.

The present disclosure provides Claudin 18.2/CD3 “imbalanced” multispecific antibodies (e.g., BsAbs), which is developed to provide an improved balance of safety/efficacy. A multi-specific antibody (e.g., bispecific antibody or a trispecific antibody) or antigen-binding fragment thereof is an artificial protein that can simultaneously bind to two or more different types of epitopes. The epitopes can be in the same antigen or in different antigens. In some embodiments, a multi-specific antibody or antigen-binding fragment thereof can have two, three, four, five, six, or more antigen binding sites. In some embodiments, the antigen binding site has one heavy chain variable region and one light chain variable region. In some embodiments, the antigen binding site has one VHH. In one aspect, the multispecific antibodies are designed to have one or more of the following features: (1) CD3 binding affinity is significantly reduced to increase safety; (2) ADCC/CDC effector functions specifically for cancer cells is maintained; (3) Biochemical and biophysical features of the two Fv arms of the IgG-like BsAbs are differentiated to enable more efficient isolation of BsAb heterodimers; (4) New Fc mutations are introduced to improve heterodimer production and purification. In vitro studies showed that the multispecific antibodies described herein were able to induce cancer specific T cell activation, tumor cell killing, and/or complement dependent cytotoxicity. Based on the data described in the disclosure, the “imbalanced” T cell engagers, e.g., Claudin 18.2/CD3 BsAbs, are promising to serve as better therapeutic antibodies for cancer treatment (e.g., for cancer expressing Claudin 18.2).

The present disclosure also provides multispecific antibodies or antigen-binding fragments thereof that binds to CD3 and one or more cancer antigens (e.g., Claudin 18.2).

CD3 (cluster of differentiation 3) is a protein complex and T cell co-receptor that is involved in activating both the cytotoxic T cell (CD8+ naive T cells) and T helper cells (CD4+ naive T cells). It is composed of four distinct chains. In mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex.

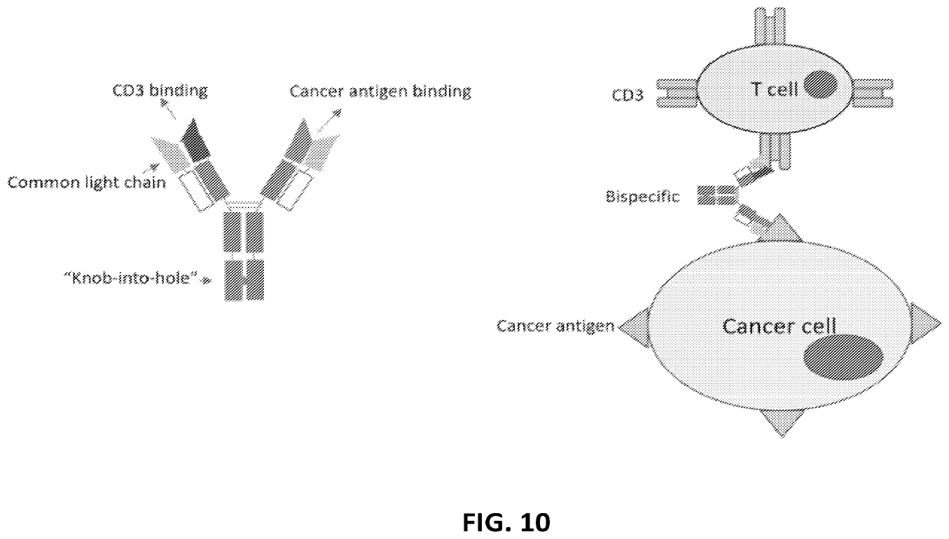

Because CD3 is involved in T cell activation, monoclonal antibodies that target it are being investigated as therapies for cancers. However, the anti-CD3 antibody by itself may activate T cells, causing uncontrolled immune response. Thus, the binding affinity with CD3 should be carefully adjusted. In many cases, the binding affinity to the cancer antigen is greater than the binding affinity to CD3. These multispecific antibodies with imbalanced affinities can have various advantages. For example, multispecific antibodies with imbalanced affinities can be used to target a cancer antigen on cancer cells and CD3 on T cell. In this case, high affinity to the cancer antigen can lead to better capturing of cancer cells by T cells, and low affinity to CD3 can avoid triggering T-cell signaling by CD3 in the absence of the cancer antigen (FIG. 10). Only when the multispecific antibodies is presented to the T cell in a multivalent fashion by a target cancer cell, can the multispecific antibodies be activated and kill the target cancer cell.

The present disclosure provides multispecific antibodies or antigen-binding fragments thereof that binds to CD3 and cancer antigens. The binding affinity to CD3 is carefully adjusted. The present disclosure further shows that the multispecific antibodies or antigen-binding fragments as described herein can only activate T cells in the presence of cells that express cancer antigens. This can greatly improve the safety and the efficacy of the multispecific antibodies. The present disclosure further provides anti-Claudin 18.2, anti-CD3 VHH and antibodies having a VH and a VL or antigen binding fragments thereof. These VHH, VH, VL can be used to make various multispecific antibodies or antigen-binding fragments as described herein.

### Claudin 18.2 and Cancer

Claudins are key structural and functional components of epithelial tight junctions, which act to regulate cell-cell permeability, maintain ion homeostasis, and support cell adhesion and polarity. Claudins are tetraspan transmembrane proteins of 22-27 kDa that multimerize within or across cell membranes to form a protective barrier. The 24 claudin proteins that have been reported differ by the specificity of their tissue localization and by their interactions with other proteins.

Claudin 18 (CLDN 18) was initially identified as a target gene for the transcription factor T/EBP/NKX2.1. Consistent with its homology to other claudin family members, CLDN18 was confirmed to localize to cellular tight junctions in mouse and human. CLDN 18 was shown to encode two isoforms generated by alternative splicing: CLDN18.1, expressed specifically in normal lung, and CLDN18.2, expressed in differentiated cells of the gastric mucosa.

CLDN18.2 is a 261 amino acid protein with two extracellular loops, and has 92% sequence identity to CLDN18.1. Unlike the second extracellular loop, the first extracellular loop of CLDN18.2 has eight amino acid differences from CLDN18.1. CLDN18.2 homology to other family members is more limited, with 29-34% overall identity to CLDN1, CLDN6 and CLDN7.

CLDN18.2 is expressed in several tumor types, including gastric cancer, pancreatic cancer, esophageal cancer, mucinous ovarian cancer and non-small cell lung cancer. CLDN18.2 expression in gastric cancer includes the invasive front and metastatic sites, although absolute levels of CLDN18 are reported to be decreased in these settings. The expression of CLDN18.2 in multiple tumor types, with normal tissue expression mainly restricted to differentiated cells in the stomach, has led to the consideration of CLDN18.2 as a therapeutic target in gastric cancer and other indications. Details can be found, e.g., in PCT/EP2019/070886; Singh, P. S. T., and Huang Y., “Anti-claudin 18.2 antibody as new targeted therapy for advanced gastric cancer.” Journal of Hematology & Oncology 10.1 (2017): 105; Zhu, G. et al., “Targeting CLDN18. 2 by CD3 bispecific and ADC modalities for the treatments of gastric and pancreatic cancer.” Scientific reports 9.1 (2019): 1-11; each of which is incorporated herein by reference.

The present disclosure also provides antibodies or antigen-binding thereof fragments (including e.g., multispecific antibodies) that binds to Claudin 18.2.

### Heavy-Chain Antibody Variable Domain (VHH)

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain antibody. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain antibody is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, nanobody, or heavy-chain antibody variable domain) is the smallest antigen-binding domain generated by adaptive immune systems. The third Complementarity Determining Region (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into multispecific antibody constructs than constructs containing conventional VH-VL pairs or single domains based on VH domains.

The disclosure provides e.g., anti-Claudin 18.2 antibodies, the modified antibodies thereof, the chimeric antibodies thereof, and the humanized antibodies thereof. The disclosure also provides VHH of these antibodies. These VHHs can be used in various multispecific antibody constructs as described herein.

The CDR sequences for Claudin 18.2-1B2 (or 1B2), and Claudin 18.2-1B2 derived antibodies (e.g., humanized antibodies) include CDRs of the VHH domain as set forth in SEQ ID NOs: 1, 2, and 3, respectively. The amino acid sequences for the VHH domain of Claudin 18.2-1B2 antibodies are set forth in SEQ ID NOs: 19, 91, and 92.

The CDR sequences for Claudin 18.2-3C2 (or 3C2), and Claudin 18.2-3C2 derived antibodies (e.g., humanized antibodies) include CDRs of the VHH domain as set forth in SEQ ID NOs: 4, 5, and 6, respectively. The amino acid sequence for the VHH domain of Claudin 18.2-3C2 antibody is set forth in SEQ ID NO: 20.

The CDR sequences for Claudin 18.2-1A1l (or lAll), and Claudin 18.2-lAll derived antibodies (e.g., humanized antibodies) include CDRs of the VHH domain as set forth in SEQ ID NOs: 7, 8, and 9, respectively. The amino acid sequences for the VHH domain of Claudin 18.2-1A11 antibodies are set forth in SEQ ID NOs: 21, 93, and 94.

The CDR sequences for Claudin 18.2-4G1 (or 4G1), and Claudin 18.2-4G1 derived antibodies (e.g., humanized antibodies) include CDRs of the VHH domain as set forth in SEQ ID NOs: 10, 11, and 12, respectively. The amino acid sequence for the VHH domain of Claudin 18.2-4G1 antibody is set forth in SEQ ID NO: 22.

The amino acid sequences for various modified or humanized VHH are also provided. As there are different ways to modify or humanize a llama antibody (e.g., a sequence can be modified with different amino acid substitutions), the heavy chain and the light chain of an antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOs: 19-22 and 91-94.

Furthermore, in some embodiments, the antibodies or antigen-binding fragments thereof described herein can also contain one, two, or three VHH domain CDRs selected from the group of SEQ ID NOs: 1-3, SEQ ID NOs: 4-6, SEQ ID NOs: 7-9, and SEQ ID NOs: 10-12.

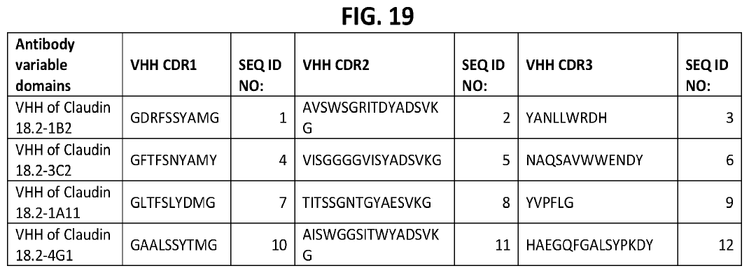

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 19.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 19.

In some embodiments, the antibody or an antigen-binding fragment described herein can contain a heavy-chain antibody variable domain (VHH) containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme.

The disclosure also provides antibodies or antigen-binding fragments thereof that bind to Claudin 18.2. The antibodies or antigen-binding fragments thereof contain a heavy-chain antibody variable domain (VHH) comprising or consisting of an amino acid sequence that is at least 80%, 85%, 90%, or 95% identical to a selected VHH sequence. In some embodiments, the selected VHH sequence is SEQ ID NO: 19. In some embodiments, the selected VHH sequence is SEQ ID NO: 20. In some embodiments, the selected VHH sequence is SEQ ID NO: 21. In some embodiments, the selected VHH sequence is SEQ ID NO: 22. In some embodiments, the selected VHH sequence is SEQ ID NO: 91. In some embodiments, the selected VHH sequence is SEQ ID NO: 92. In some embodiments, the selected VHH sequence is SEQ ID NO: 93. In some embodiments, the selected VHH sequence is SEQ ID NO: 94.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

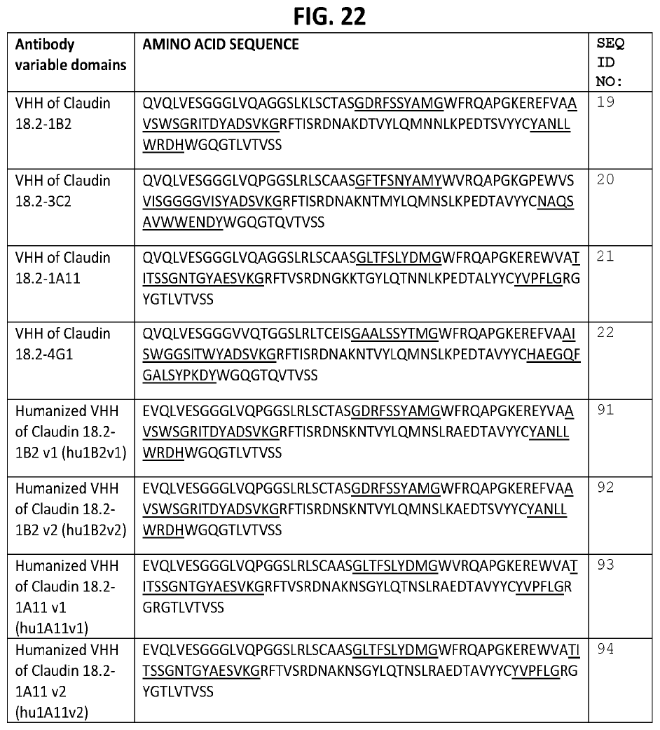

The disclosure also provides nucleic acid comprising a polynucleotide encoding a polypeptide comprising an immunoglobulin heavy-chain antibody variable domain (VHH). The VHH comprises CDRs as shown in FIG. 19, or has sequences as shown in FIG. 22.

The antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof.

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

The disclosure also provides antibodies or antigen-binding fragments thereof that bind to Claudin 18.2. In some embodiments, the antibody or antigen-binding fragment thereof comprises a heavy-chain antibody variable domain (VHH) CDR1 selected from SEQ ID NO: 1, 4, 7, or 10. In some embodiments, the antibody or antigen-binding fragment thereof comprises a heavy-chain antibody variable domain (VHH) CDR2 selected from SEQ ID NO: 2, 5, 8, or 11. In some embodiments, the antibody or antigen-binding fragment thereof comprises a heavy-chain antibody variable domain (VHH) CDR3 selected from SEQ ID NO: 3, 6, 9, or 12.

### Anti-CD3 Antibodies and Antigen-Binding Fragments

The disclosure provides antibodies and antigen-binding fragments thereof that specifically bind to CD3. The antibodies and antigen-binding fragments described herein are capable of binding to CD3. These antibodies can be agonists or antagonists. In some embodiments, these antibodies can promote CD3-associated signaling pathway (e.g., antigen presenting to T cells) thus increase immune response. In some embodiments, these antibodies can initiate CMC or ADCC.

The disclosure provides e.g., anti-CD3 antibody, the chimeric antibodies thereof, and the humanized antibodies thereof. In some embodiments, the CDR sequences for the anti-CD3 antibody, and its derived antibodies (e.g., humanized antibodies) include CDRs of the heavy chain variable domain, SEQ ID NOs: 13, 14, and 15. The VH with these VH CDRs can be paired with VLs with various different VL CDRs. In some embodiments, the CDR sequences for anti-CD3 antibody, and its derived antibodies (e.g., humanized antibodies) include CDRs of the light chain variable domain, SEQ ID NOs: 16, 17, and 18.

The amino acid sequences for heavy chain variable regions and light variable regions of the humanized antibodies are also provided. As there are different ways to humanize a mouse antibody (e.g., a sequence can be modified with different amino acid substitutions), the heavy chain and the light chain of an antibody can have more than one version of humanized sequences. In some embodiments, the humanized heavy chain variable region (VH) is at least 80%, 85%, 90%, 91%, 92%, 93%, 94%, 95%, 96%, 97%, 98%, or 99% identical to SEQ ID NO: 23 or 95. In some embodiments, the humanized light chain variable region (VL) is at least 80%, 85%, 90%, 91%, 92%, 93%, 94%, 95%, 96%, 97%, 98%, or 99% identical to SEQ ID NO: 24 or 96.

In some embodiments, the amino acid sequence for the heavy chain variable region of the anti-CD3 antibody is set forth in SEQ ID NO: 23, and the amino acid sequence for the light chain variable regions of the anti-CD3 antibody is set forth in SEQ ID NO: 24. In some embodiments, the amino acid sequence for the heavy chain variable region of the anti-CD3 antibody is set forth in SEQ ID NO: 95, and the amino acid sequence for the light chain variable regions of the anti-CD3 antibody is set forth in SEQ ID NO: 96.

Furthermore, in some embodiments, the antibodies or antigen-binding fragments thereof described herein can also contain one, two, or three heavy chain variable region CDRs selected from the group of SEQ ID NOs: 13-15; and/or one, two, or three light chain variable region CDRs selected from the group of SEQ ID NOs: 16-18.

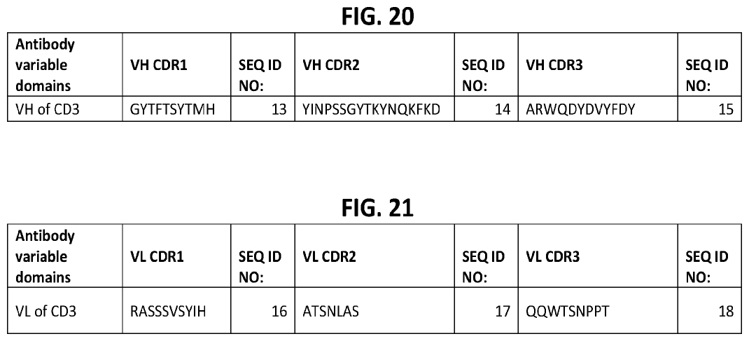

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 20 (VH CDR) and FIG. 21 (VL CDR).

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a light chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme.

The disclosure also provides antibodies or antigen-binding fragments thereof that bind to CD3. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 23, and the selected VL sequence is SEQ ID NO: 24. In some embodiments, the selected VH sequence is SEQ ID NO: 95, and the selected VL sequence is SEQ ID NO: 96.

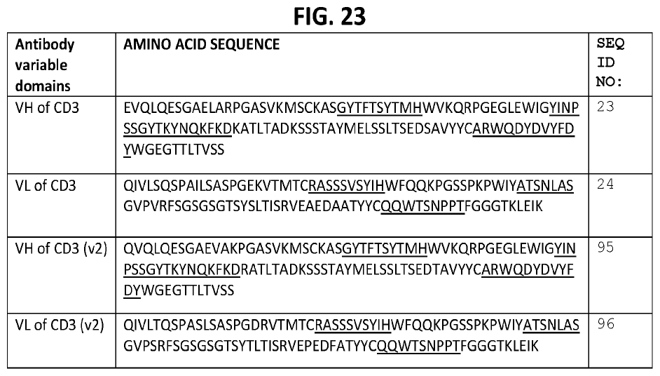

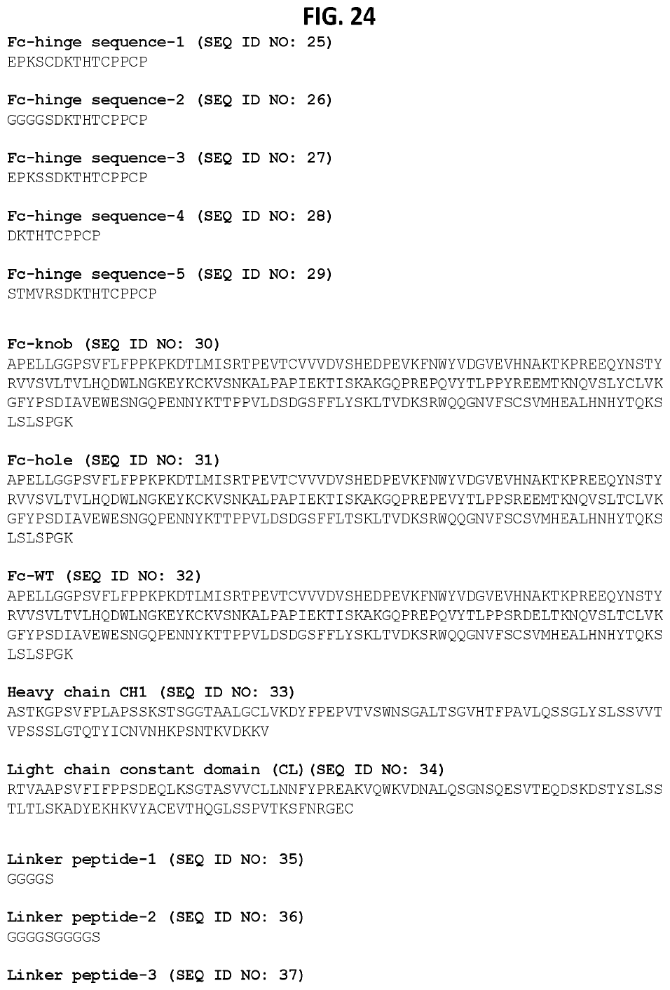

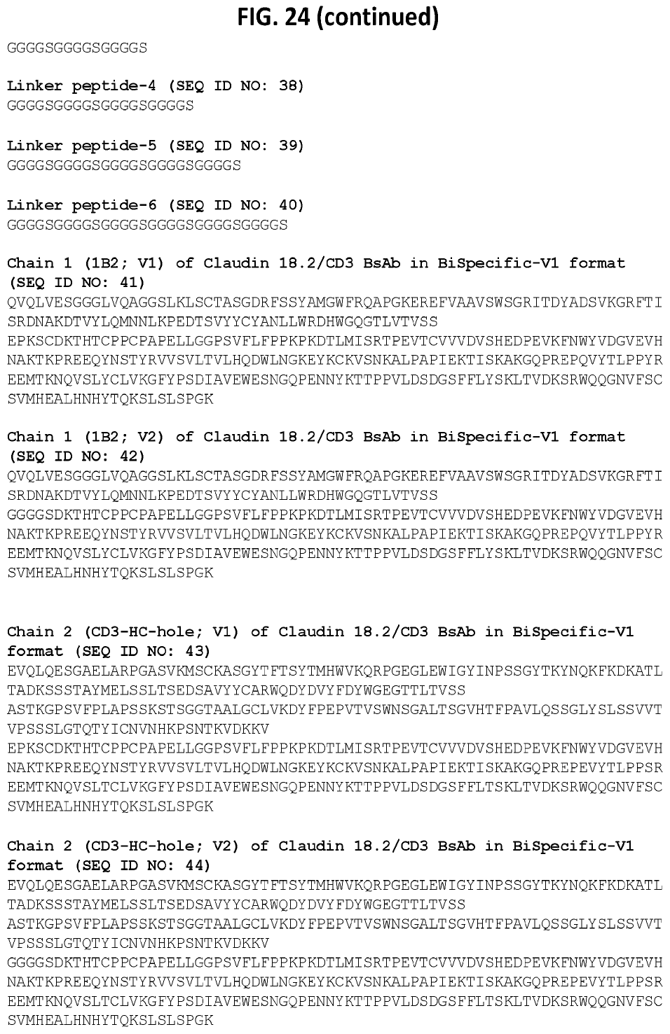

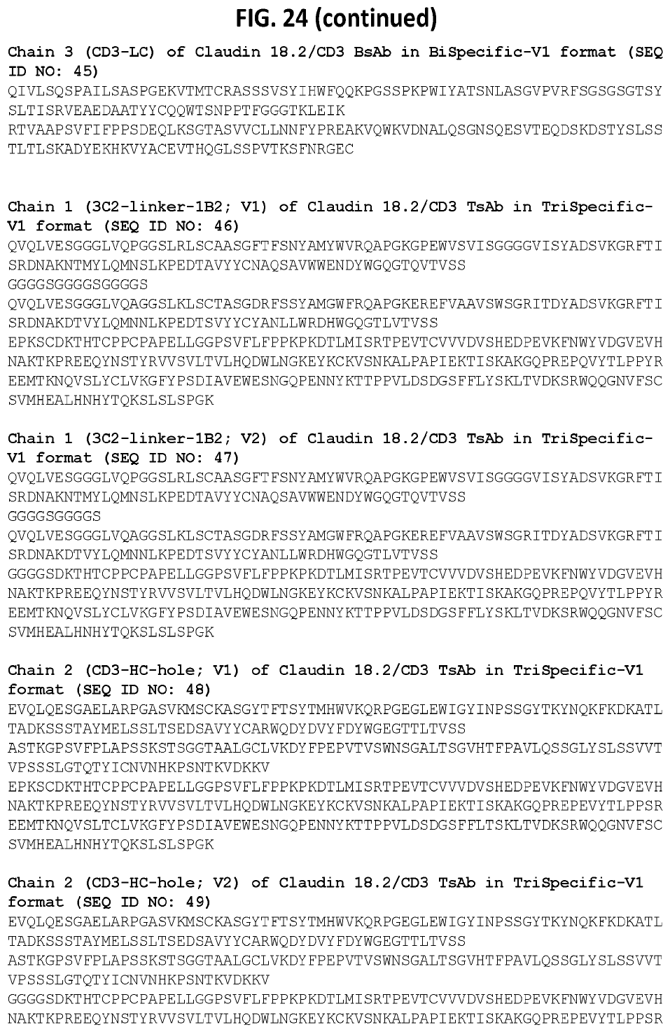

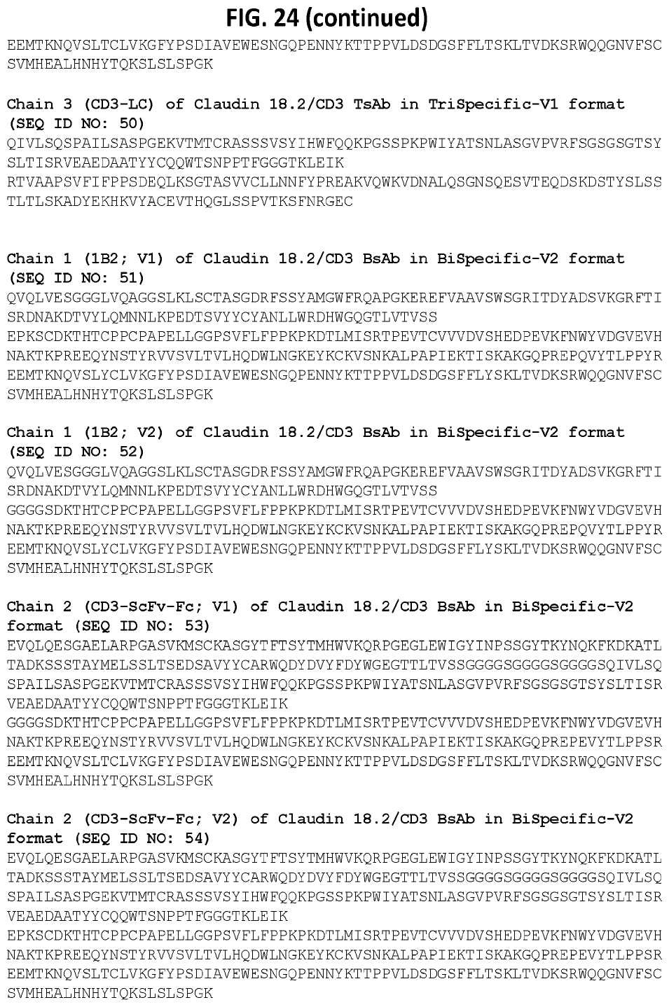

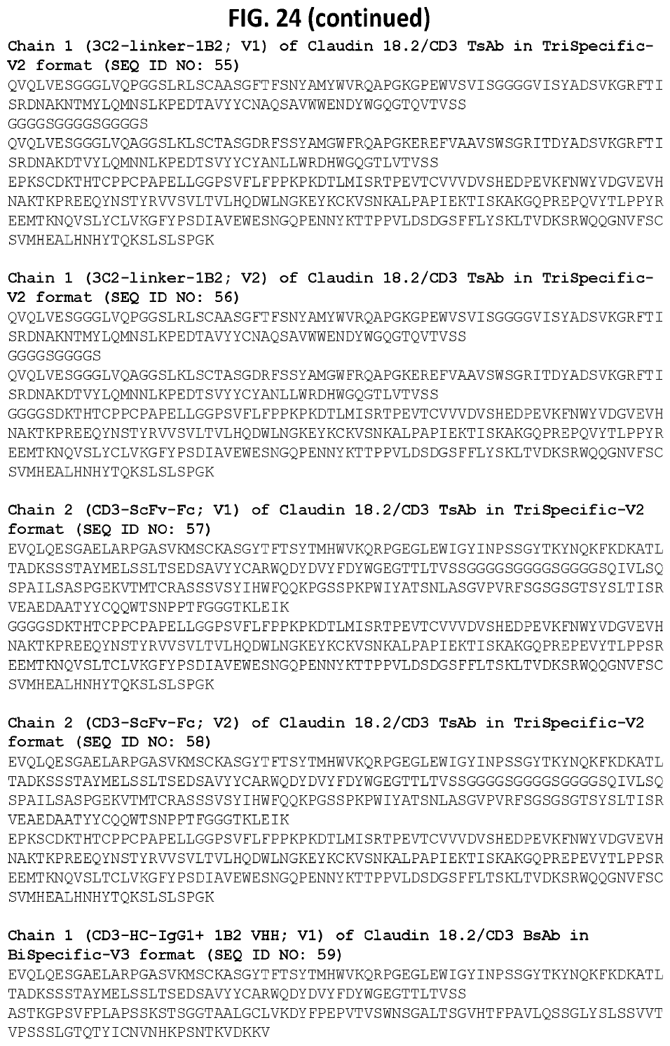

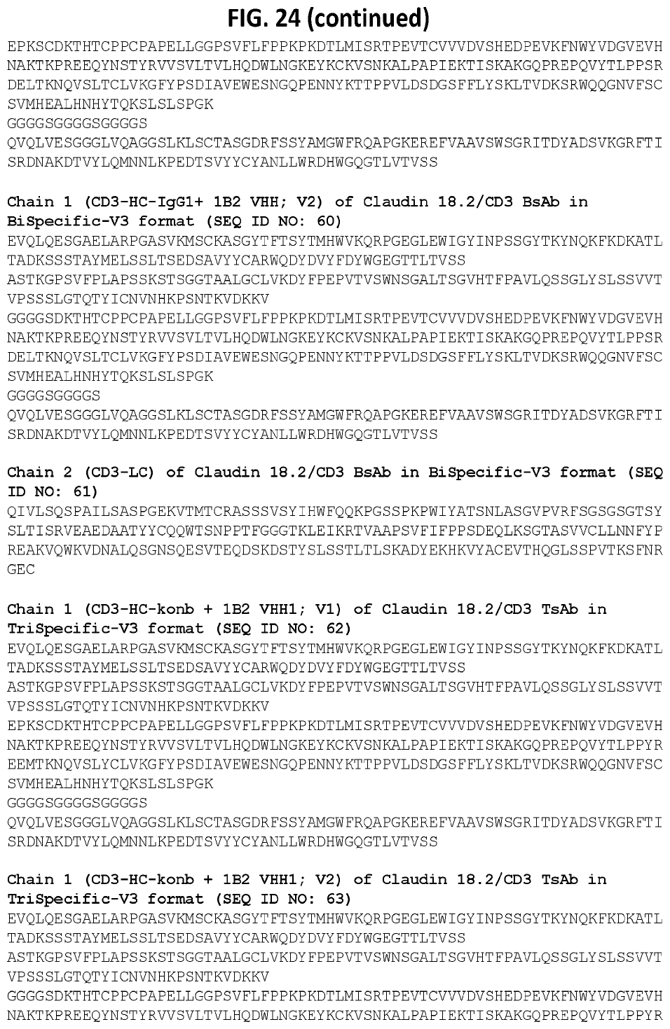

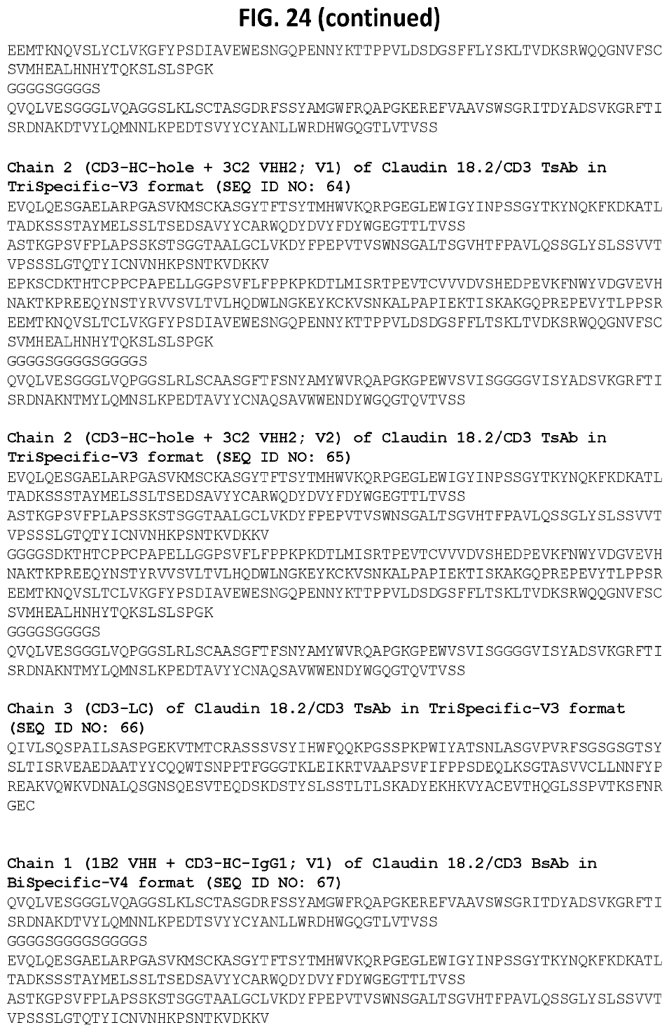

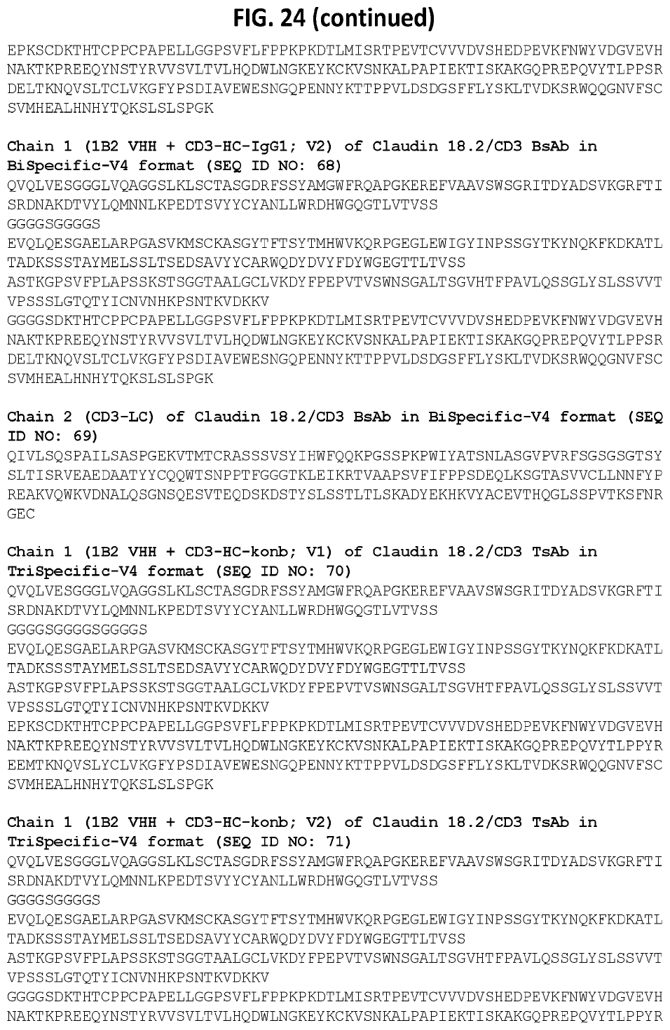

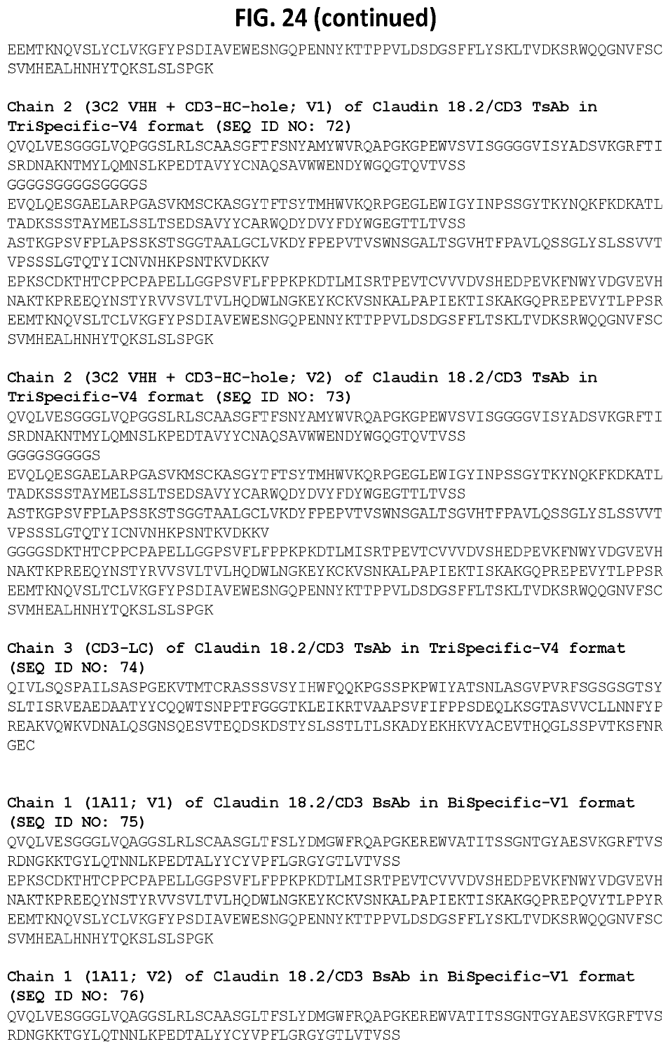

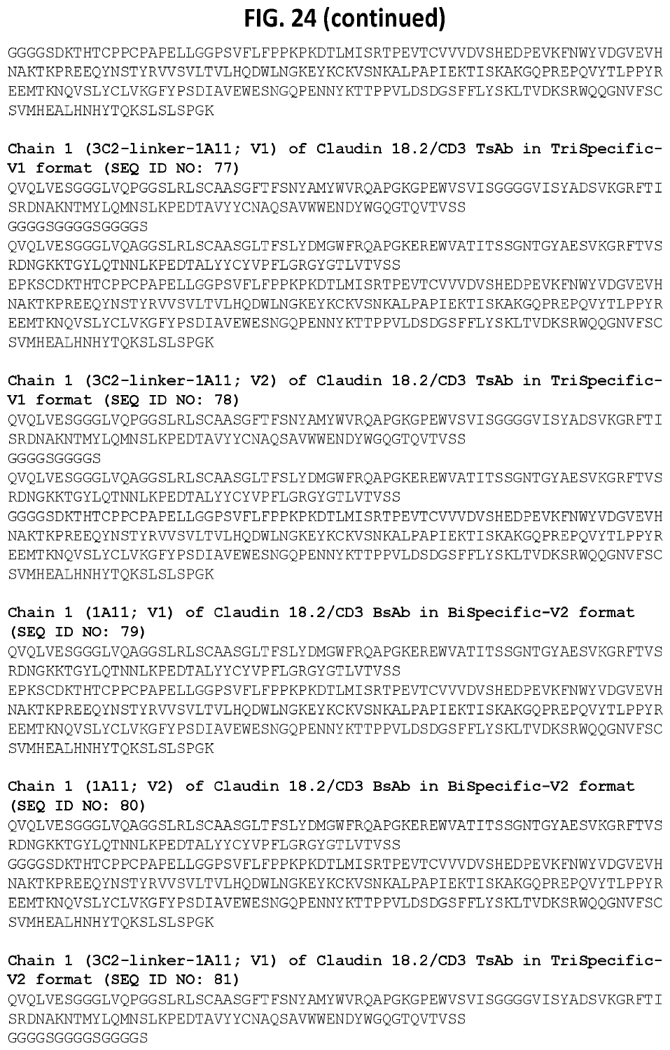

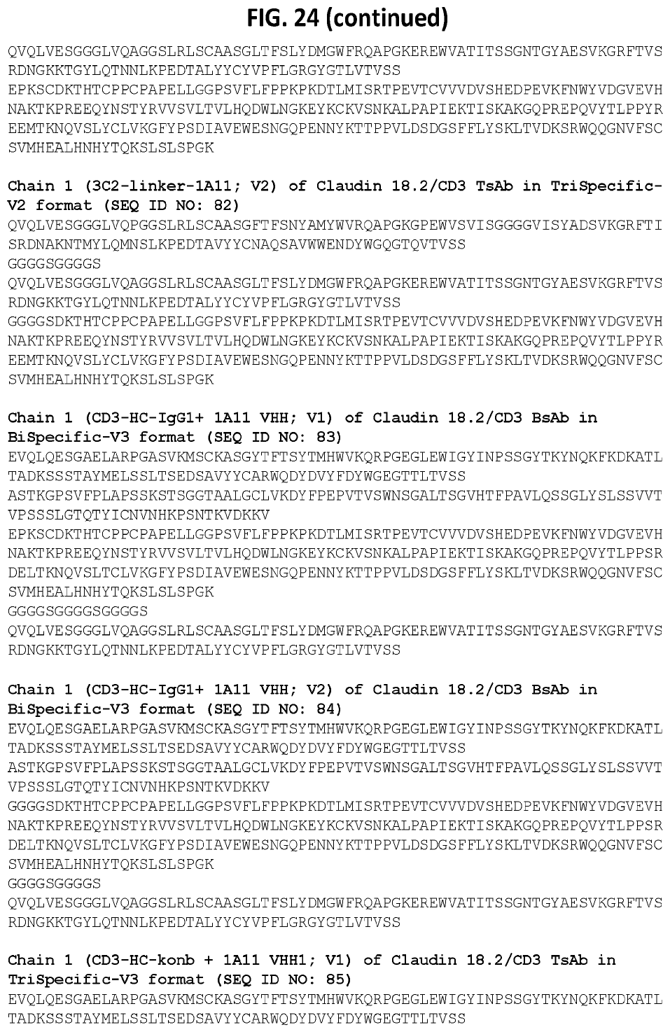

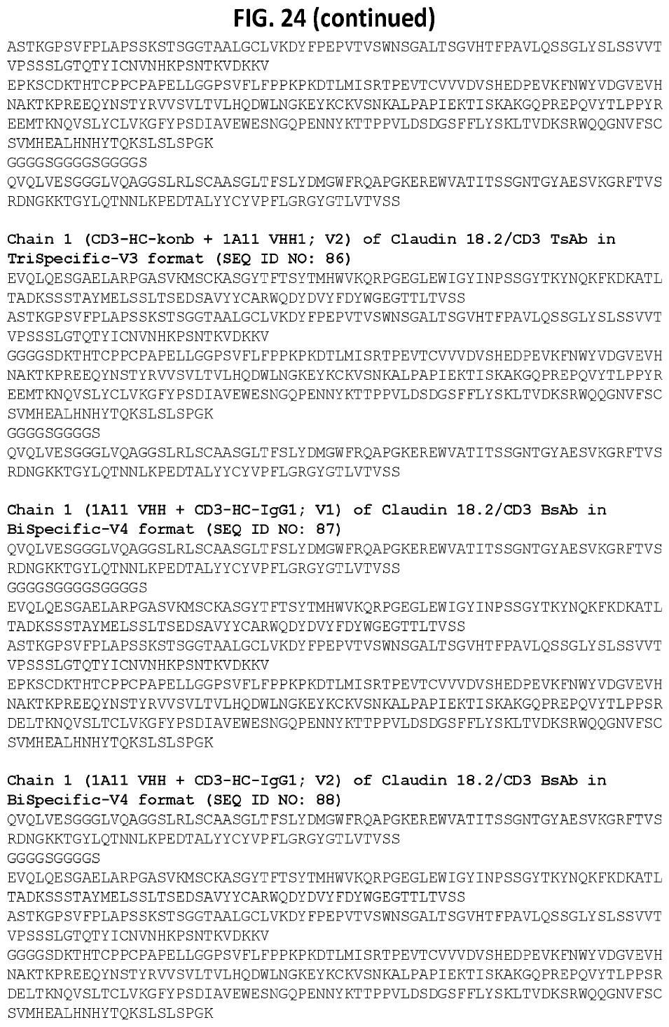

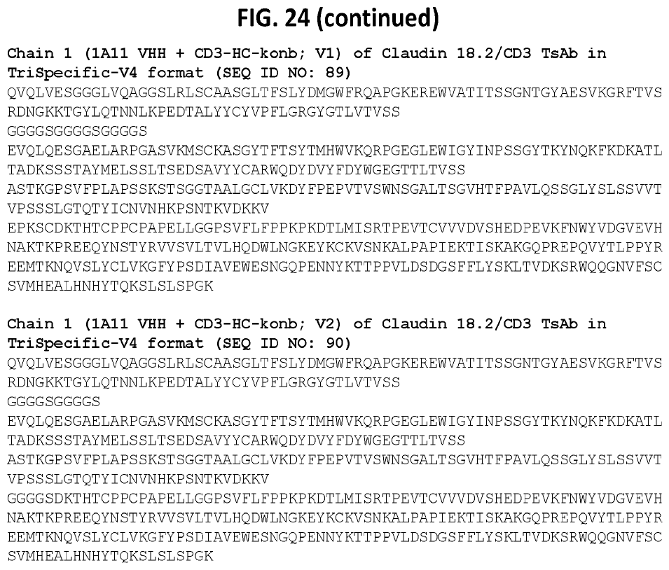

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 20 and FIG. 21, respectively, or have sequences as shown in FIG. 23. When the polypeptides are paired with corresponding polypeptide (e.g., a corresponding heavy chain variable region or a corresponding light chain variable region), the paired polypeptides bind to CD3 (e.g., human CD3).

The anti-CD3 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to CD3 will retain an ability to bind to CD3.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 24 or 96, binds to CD3; or
(2) an immunoglobulin light chain or a fragment thereof comprising a VL comprising CDRs 1, 2, and 3 comprising the amino acid sequences set forth in SEQ ID NOs: 16, 17, and 18, respectively, and wherein the VL, when paired with a VH comprising the amino acid sequence set forth in SEQ ID NO: 23 or 95, binds to CD3.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 16, 17, and 18, respectively.

In some embodiments, the VH when paired with a VL specifically binds to human CD3, or the VL when paired with a VH specifically binds to human CD3.

In some embodiments, the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof.

In some embodiments, the nucleic acid encodes a single-chain variable fragment (scFv). In some embodiments, the nucleic acid is cDNA.

In one aspect, the disclosure is related to a vector comprising one or more of the nucleic acids described herein. In one aspect, the disclosure is related to a vector comprising two of the nucleic acids described herein, wherein the vector encodes the VL region and the VH region that together bind to CD3. In one aspect, the disclosure is related to a pair of vectors, wherein each vector comprises one of the nucleic acids described herein, wherein together the pair of vectors encodes the VL region and the VH region that together bind to CD3.

In one aspect, the disclosure is related to a cell comprising the vector or the pair of vectors described herein. In one aspect, the disclosure is related to two of the nucleic acids described herein.

In some embodiments, the two nucleic acids together encode the VL region and the VH region that together bind to CD3.

Imbalanced Multi-Specific Antibodies that Bind to T Cell Specific Antigen and Cancer Antigens

Multispecific antibodies (e.g., bispecific antibody) with a T cell specific antigen (e.g., CD3, CD4, or CD8) binding site that can recruit and activate T cells. Because an antibody's effector function such as ADCC and CDC have been shown to play a critical role in cancer cell killing, “safely” maintaining an antibody's effector function would expand the mechanisms of action of an therapeutic antibody as well as improve the antibody's cancer killing function. To “safely” maintain the effector functions and expand the applications of these bispecific antibodies, various multispecific antibodies have been developed.

In an exemplary design shown in FIG. 10, the first antigen binding region targets a cancer antigen (e.g., Claudin 18.2), and the second antigen binding region targets a T cell specific antigen, i.e., CD3, to recruit T cell to attack cancer with the cancer antigen. The term “cancer antigen” refers to antigens that are primarily expressed on cancer cell surfaces. These antigens can be used to identify cancer or tumor cells. Normal cells rarely express cancer antigens.

An antibody with high affinity to CD3 can trigger T-cell signaling, and cause undesirable immune response. Thus, a low affinity (e.g., KD can be greater than 10⁻⁵ M, 10⁻⁶ M, 10⁻⁷ M, 10⁻⁸ M, or 10⁻⁹ M) to CD3 is required to reduce the risk of triggering T-cell signaling by CD3 while “safely” maintaining the antibody's effector function. As used herein, the term “safely maintaining the antibody's effector function” means that the antibody does not induce ADCC or CDC on normal cells (e.g., non-cancer cells). When multiple bispecific antibodies are presented on a target cancer cells (e.g., in a cluster) and bridge the interaction between cancer cell and T cell, these bispecific antibodies can trigger T-cell signaling though CD3 in a multivalent fashion, and the activated T cells will then kill the target cancer cells. Because the multispecific antibody applies different mechanism of action to treat cancer compared to therapeutic antibodies that target a cancer specific antigen alone, in some embodiments, it can be used as an alternative therapy for therapeutic monoclonal antibodies that target a cancer specific antigen, especially for those cancers which don not respond well to therapeutic monoclonal antibodies that only targets a cancer specific antigen.

In some embodiments, multi-specific (e.g., trispecific) antibodies are designed that include an additional antigen binding region that targets a cancer antigen (e.g., Claudin 18.2). The multispecific (e.g., bispecific and trispecific) antibodies are described below.

Claudin 18.2 belongs to the large claudin family of proteins, which form tight junction strands in epithelial cells. As Claudin 18.2 is abundant in solid cancers, e.g., pancreatic and gastric cancers, Claudin 18.2 is a cancer antigen. The present disclosure provides multi-specific (e.g., bispecific or trispecific) antibodies that bind to both Claudin 18.2 and CD3. The bispecific or trispecific antibodies can be used to treat Claudin 18.2 positive cancers (e.g., pancreatic and gastric adenocarcinoma) in a subject.

The Claudin 18.2/CD3 bispecific and trispecific antibodies with specific structures are described below.

### BiSpecific-V1 TriSpecific-V1 Structures

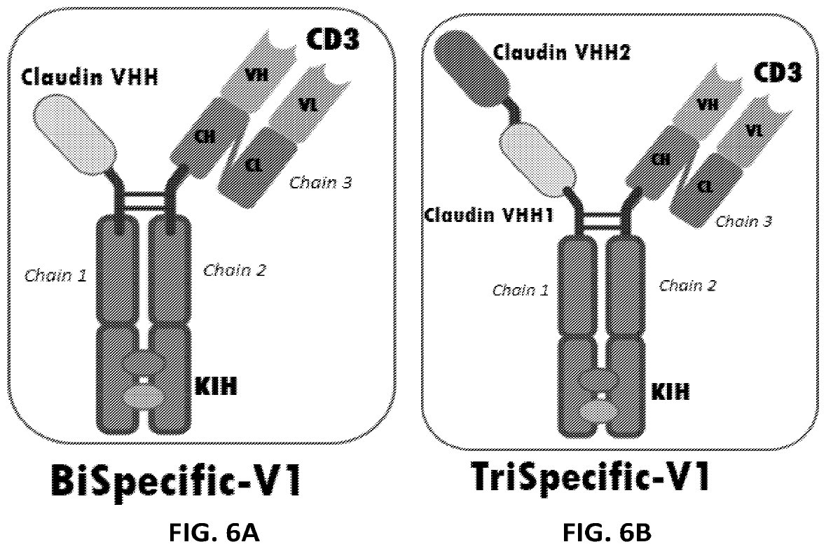

As shown in FIG. 6A, a Claudin 18.2/CD3 bispecific antibody can be prepared to have a BiSpecific-V1 structure. Specifically, the Claudin 18.2/CD3 bispecific antibody comprises (a) a first polypeptide comprising from N-terminus to C-terminus: a first heavy-chain antibody variable domain (VHH), a first hinge region, a first Fc region; (b) a second polypeptide comprising from N-terminus to C-terminus: a heavy chain variable region (VH), a second hinge region, and a second Fc region; and (c) a third polypeptide comprising a light chain variable region (VL). In some embodiments, the first VHH specifically binds to Claudin 18.2, wherein the VH and the VL associate with each other and specifically bind to CD3.

In some embodiments, the first hinge region and/or the second hinge region comprise a sequence that is at least 80%, 85%, 90%, 95%, or 100% identical to any one of SEQ ID NOs: 25-29. In some embodiments, the first Fc region and/or the second Fc region comprise a sequence that is at least 80%, 85%, 90%, 95%, or 100% identical to SEQ ID NO: 30 or 31.

In some embodiments, the Claudin 18.2/CD3 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 41,42, 75, or 76. In some embodiments, the second polypeptide comprises a sequence that is at least 80%, 85%, 90%, 95%, or 100% identical to SEQ ID NO: 43 or 44. In some embodiments, the third polypeptide comprises a sequence that is at least 80%, 85%, 90%, 95%, or 100% identical to SEQ ID NO: 45.

As shown in FIG. 6B, a Claudin 18.2/CD3 trispecific antibody can be prepared to have a TriSpecific-V1 structure. Specifically, the first polypeptide as described in the BiSpecific-V1 structure can further include a second VHH that specifically binds to Claudin 18.2, wherein the second VHH is linked to the N-terminus of the first VHH via a linker peptide sequence. In some embodiments, the linker peptide sequence comprises a sequence that is at least 80%, 85%, 90%, 95%, or 100% identical to any one SEQ ID NOs: 35-40. In some embodiments, the linker peptide sequence comprises a sequence that is at least 80% identical to SEQ ID NO: 36 or 37. In some embodiments, the linker peptide sequence comprises a sequence that is at least 80%, 85%, 90%, 95%, or 100% identical to one or more (e.g., 1, 2, 3, 4, 5, 6, 7, or 8) repeats of GGGGS (SEQ ID NO: 35).

In some embodiments, the first polypeptide comprises a sequence that is at least 80%, 85%, 90%, 95%, or 100% identical to SEQ ID NO: 46,47, 77, or 78. In some embodiments, the second polypeptide comprises a sequence that is at least 80%, 85%, 90%, 95%, or 100% identical to SEQ ID NO: 48 or 49. In some embodiments, the third polypeptide comprises a sequence that is at least 80%, 85%, 90%, 95%, or 100% identical to SEQ ID NO: 50.

In some embodiments, the third polypeptide comprises a sequence that is at least 80%, 85%, 90%, 95%, or 100% identical to SEQ ID NO: 34.

### BiSpecific-V2 TriSpecific-V2 Structures

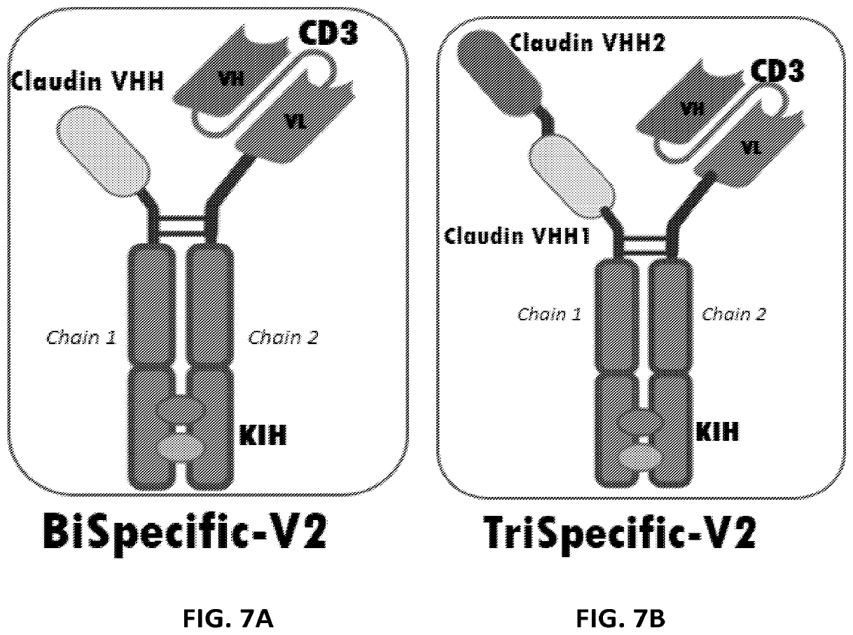

As shown in FIG. 7A, a Claudin 18.2/CD3 bispecific antibody can be prepared to have a BiSpecific-V2 structure. Specifically, the Claudin 18.2/CD3 bispecific antibody comprises (a) a first polypeptide comprising from N-terminus to C-terminus: a first heavy-chain antibody variable domain (VHH), a first hinge region, a first Fc region; and (b) a second polypeptide comprising from N-terminus to C-terminus: a single-chain variable fragment (scFv), a second hinge region, and a second Fc region.

In some embodiments, the first VHH specifically binds to Claudin 18.2. In some embodiments, the scFv comprises a heavy chain variable region (VH), a first linker peptide sequence, and a light chain variable region (VL). In some embodiments, the VH and the VL associate with each other and specifically bind to CD3.

In some embodiments, the first linker peptide sequence comprises a sequence that is at least 80%, 85%, 90%, 95%, or 100% identical to any one SEQ ID NOs: 35-40. In some embodiments, the first linker peptide sequence comprises a sequence that is at least 80%, 85%, 90%, 95%, or 100% identical to SEQ ID NO: 36 or 37. In some embodiments, the first hinge region and/or the second hinge region comprise a sequence that is at least 80%, 85%, 90%, 95%, or 100% identical to any one of SEQ ID NOs: 25-29. In some embodiments, the first Fc region and/or the second Fc region comprise a sequence that is at least 80%, 85%, 90%, 95%, or 100% identical to SEQ ID NO: 30 or 31.

In some embodiments, the Claudin 18.2/CD3 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 51,52, 79, or 80. In some embodiments, the second polypeptide comprises a sequence that is at least 80%, 85%, 90%, 95%, or 100% identical to SEQ ID NO: 53 or 54.

As shown in FIG. 7B, a Claudin 18.2/CD3 trispecific antibody can be prepared to have a TriSpecific-V2 structure. Specifically, the first polypeptide as described in the BiSpecific-V2 structure can further include a second VHH that specifically binds to Claudin 18.2, wherein the second VHH is linked to the N-terminus of the first VHH via a second linker peptide sequence.

In some embodiments, the second linker peptide sequence comprises a sequence that is at least 80%, 85%, 90%, 95%, or 100% identical to any one SEQ ID NOs: 35-40. In some embodiments, the second linker peptide sequence comprises a sequence that is at least 80%, 85%, 90%, 95%, or 100% identical to SEQ ID NO: 36 or 37. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 35).

In some embodiments, the first polypeptide comprises a sequence that is at least 80%, 85%, 90%, 95%, or 100% identical to SEQ ID NO: 55,56, 81, or 82. In some embodiments, the second polypeptide comprises a sequence that is at least 80% identical to SEQ ID NO: 57 or 58.

### BiSpecific-V3 TriSpecific-V3 Structures

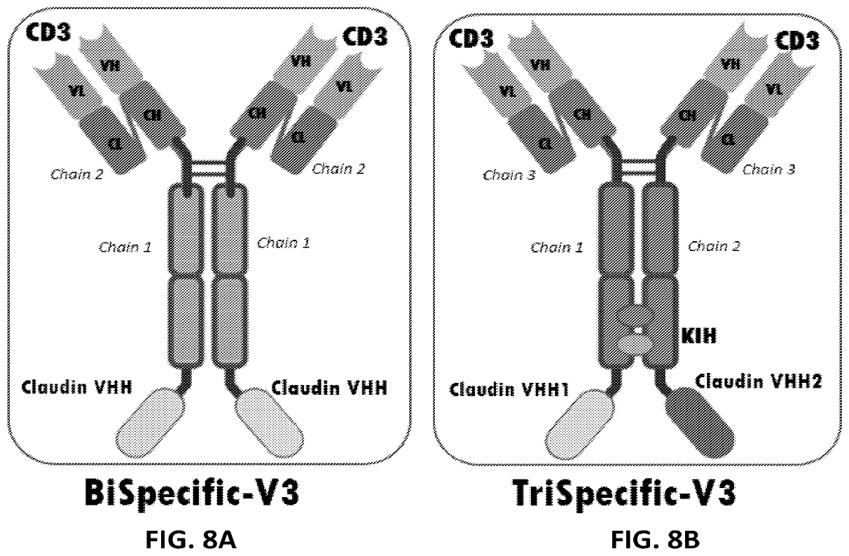

As shown in FIG. 8A, a Claudin 18.2/CD3 bispecific antibody can be prepared to have a BiSpecific-V3 structure. Specifically, the Claudin 18.2/CD3 bispecific antibody comprises (a) a first polypeptide comprising from N-terminus to C-terminus: a first heavy chain variable region (VH1), a first hinge region, a first Fc region; a first linker peptide sequence, and a first heavy-chain antibody variable domain (VHH1); (b) a second polypeptide comprising a first light chain variable region (VL1); (c) a third polypeptide comprising from N-terminus to C-terminus: a second heavy chain variable region (VH2), a second hinge region, a second Fc region, a second linker peptide sequence, and a second heavy-chain antibody variable domain (VHH2); and (d) a fourth polypeptide comprising a second light chain variable region (VL2). In some embodiments, the VHH1 and/or the VHH2 specifically bind to Claudin 18.2. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80%, 85%, 90%, 95%, or 100% identical to any one SEQ ID NOs: 35-40. In some embodiments, the first linker peptide sequence and/or the second linker peptide sequence comprises a sequence that is at least 80%, 85%, 90%, 95%, or 100% identical to SEQ ID NO: 36 or 37. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 35).

In some embodiments, the first hinge region and/or the second hinge regions comprise a sequence that is at least 80%, 85%, 90%, 95%, or 100% identical to any one of SEQ ID NOs: 25-29.

In some embodiments, sequences of the VHH1 and the VHH2 are identical. In some embodiments, the first Fc region and/or the second Fc region comprise a sequence that is at least 80%, 85%, 90%, 95%, or 100% identical to SEQ ID NO: 32. In some embodiments, the first polypeptide comprises a sequence that is at least 80%, 85%, 90%, 95%, or 100% identical to SEQ ID NO: 59,60, 83, or 84. In some embodiments, the second polypeptide comprises a sequence that is at least 80%, 85%, 90%, 95%, or 100% identical to SEQ ID NO: 61. In some embodiments, the third polypeptide comprises a sequence that is at least 80%, 85%, 90%, 95%, or 100% identical to SEQ ID NO: 59,60, 83, or 84. In some embodiments, the fourth polypeptide comprises a sequence that is at least 80%, 85%, 90%, 95%, or 100% identical to SEQ ID NO: 61.

As shown in FIG. 8B, a Claudin 18.2/CD3 trispecific antibody can be prepared to have a TriSpecific-V3 structure. In some embodiments, sequences of the VHH1 and the VHH2 as described in the BiSpecific-V3 structure are different. In some embodiments, the first Fc region and/or the second Fc region comprise a sequence that is at least 80%, 85%, 90%, 95%, or 100% identical to SEQ ID NO: 30 or 31. In some embodiments, the first polypeptide comprises a sequence that is at least 80%, 85%, 90%, 95%, or 100% identical to SEQ ID NO: 62,63, 85, or 86. In some embodiments, the second polypeptide comprises a sequence that is at least 80%, 85%, 90%, 95%, or 100% identical to SEQ ID NO: 66. In some embodiments, the third polypeptide comprises a sequence that is at least 80%, 85%, 90%, 95%, or 100% identical to SEQ ID NO: 64 or 65. In some embodiments, the fourth polypeptide comprises a sequence that is at least 80%, 85%, 90%, 95%, or 100% identical to SEQ ID NO: 66.

In some embodiments, the Fc region is an IgG1 (e.g., human IgG1) Fc region. In some embodiments, the Claudin 18.2/CD3 bispecific or trispecific antibody described herein comprises knob-into-hole mutations.

In some embodiments, the second polypeptide and/or the fourth polypeptide comprise a sequence that is at least 80%, 85%, 90%, 95%, or 100% identical to SEQ ID NO: 34.

### BiSpecific-V4 TriSpecific-V4 Structures

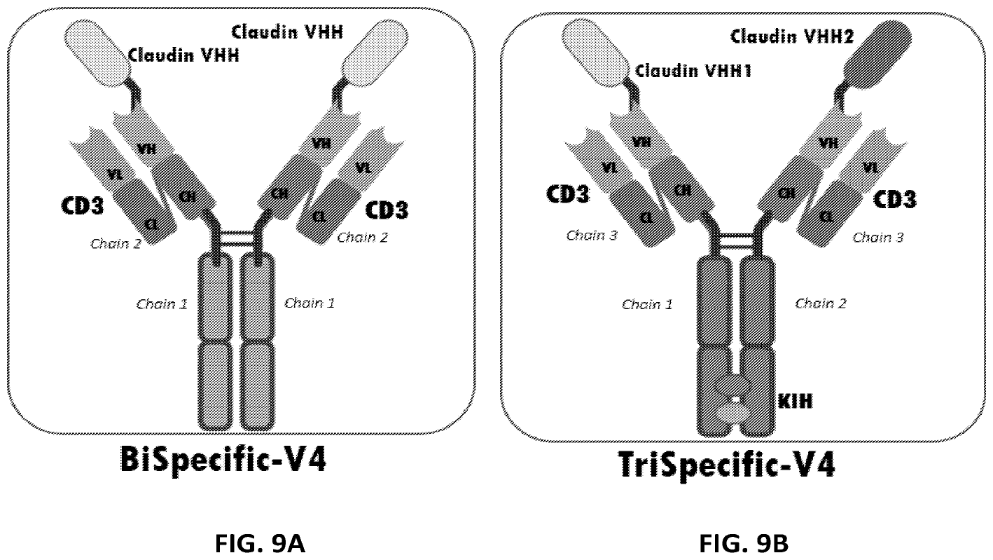

As shown in FIG. 9A, a Claudin 18.2/CD3 bispecific antibody can be prepared to have a BiSpecific-V4 structure. Specifically, the Claudin 18.2/CD3 bispecific antibody comprises (a) a first polypeptide comprising from N-terminus to C-terminus: a first heavy-chain antibody variable domain (VHH1), a first linker peptide sequence, a first heavy chain variable region (VH1), a first hinge region, and a first Fc region; (b) a second polypeptide comprising a first light chain variable region (VL1); (c) a third polypeptide comprising from N-terminus to C-terminus: a second heavy-chain antibody variable domain (VHH2), a second linker peptide sequence, a second heavy chain variable region (VH2), a second hinge region, and a second Fc region; and (d) a fourth polypeptide comprising a second light chain variable region (VL2). In some embodiments, the VHH1 and the VHH2 specifically bind to Claudin 18.2. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80%, 85%, 90%, 95%, or 100% identical to any one SEQ ID NOs: 35-40. In some embodiments, the first linker peptide sequence and/or the second linker peptide sequence comprises a sequence that is at least 80% identical to SEQ ID NO: 36 or 37. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 35).

In some embodiments, the first hinge region and/or the second hinge regions comprise a sequence that is at least 80%, 85%, 90%, 95%, or 100% identical to any one of SEQ ID NOs: 25-29.

In some embodiments, sequences of the VHH1 and the VHH2 are identical. In some embodiments, the first Fc region and/or the second Fc region comprise a sequence that is at least 80%, 85%, 90%, 95%, or 100% identical to SEQ ID NO: 32. In some embodiments, the first polypeptide comprises a sequence that is at least 80%, 85%, 90%, 95%, or 100% identical to SEQ ID NO: 67,68, 87, or 88. In some embodiments, the second polypeptide comprises a sequence that is at least 80%, 85%, 90%, 95%, or 100% identical to SEQ ID NO: 69. In some embodiments, the third polypeptide comprises a sequence that is at least 80%, 85%, 90%, 95%, or 100% identical to SEQ ID NO: 67,68, 87, or 88. In some embodiments, the fourth polypeptide comprises a sequence that is at least 80%, 85%, 90%, 95%, or 100% identical to SEQ ID NO: 69.

As shown in FIG. 9B, a Claudin 18.2/CD3 trispecific antibody can be prepared to have a TriSpecific-V4 structure. In some embodiments, sequences of the VHH1 and the VHH2 as described in the BiSpecific-V4 structure are different. In some embodiments, the first Fc region and/or the second Fc region comprise a sequence that is at least 80%, 85%, 90%, 95%, or 100% identical to SEQ ID NO: 30 or 31. In some embodiments, the first polypeptide comprises a sequence that is at least 80%, 85%, 90%, 95%, or 100% identical to SEQ ID NO: 70,71, 89, or 90. In some embodiments, the second polypeptide comprises a sequence that is at least 80%, 85%, 90%, 95%, or 100% identical to SEQ ID NO: 74. In some embodiments, the third polypeptide comprises a sequence that is at least 80%, 85%, 90%, 95%, or 100% identical to SEQ ID NO: 72 or 73. In some embodiments, the fourth polypeptide comprises a sequence that is at least 80%, 85%, 90%, 95%, or 100% identical to SEQ ID NO: 74.

In some embodiments, the Fc region is an IgG1 (e.g., human IgG1) Fc region. In some embodiments, the Claudin 18.2/CD3 bispecific or trispecific antibody described herein comprises knob-into-hole mutations.

In some embodiments, the second polypeptide and/or the fourth polypeptide comprise a sequence that is at least 80%, 85%, 90%, 95%, or 100% identical to SEQ ID NO: 34.

In some embodiments, the VHH that specifically binds to Claudin 18.2 of the Claudin 18.2/CD3 bispecific antibody with the BiSpecific-V1, BiSpecific-V2, BiSpecific-V3, or BiSpecific-V4 structure can be selected from any of the VHHs targeting Claudin 18.2 described in the disclosure. In some embodiments, the VHH that specifically binds to Claudin 18.2 of the Claudin 18.2/CD3 trispecific antibody with the TriSpecific-V1, TriSpecific-V2, TriSpecific-V3, or TriSpecific-V4 structure can be selected from any of the VHHs targeting Claudin 18.2 described in the disclosure.

In some embodiments, the VH, when associated with a VL, that specifically binds to CD3 of the Claudin 18.2/CD3 bispecific antibody with the BiSpecific-V1, BiSpecific-V2, BiSpecific-V3, or BiSpecific-V4 structure can be selected from any of the VH targeting CD3 described in the disclosure. In some embodiments, the VH, when associated with a VL, that specifically binds to CD3 of the Claudin 18.2/CD3 trispecific antibody with the TriSpecific-V1, TriSpecific-V2, TriSpecific-V3, or TriSpecific-V4 structure can be selected from any of the VH targeting CD3 described in the disclosure.

In some embodiments, the VL, when associated with a VH, that specifically binds to CD3 of the Claudin 18.2/CD3 bispecific antibody with the BiSpecific-V1, BiSpecific-V2, BiSpecific-V3, or BiSpecific-V4 structure can be selected from any of the VL targeting CD3 described in the disclosure. In some embodiments, the VL, when associated with a VH, that specifically binds to CD3 of the Claudin 18.2/CD3 trispecific antibody with the TriSpecific-V1, TriSpecific-V2, TriSpecific-V3, or TriSpecific-V4 structure can be selected from any of the VL targeting CD3 described in the disclosure.

### Antibody Characteristics

The anti-Claudin 18.2, anti-CD3, or anti-Claudin 18.2/CD3 antigen-binding protein construct (e.g., antibodies, bispecific antibodies, trispecific antibodies, multi-specific antibodies, or antibody fragments thereof) can include an antigen binding site that is derived from any anti-Claudin 18.2 antibody, anti-CD3 antibody, or any antigen-binding fragment thereof as described herein.

In some embodiments, the antibodies, or antigen-binding fragments thereof described herein can bind to Claudin 18.2 and CD3, thereby bridging the interaction between cancer cell (e.g., Claudin 18.2-expressing cancer cell) and T cell.

In some embodiments, the antibodies, or antigen-binding fragments thereof described herein can bind to Claudin 18.2-expressing cells (e.g., MIA PaCa-2 cells). In some embodiments, the antibodies, or antigen-binding fragments thereof described herein can bind to CD3-expressing cells (e.g., Jurkat cells or PBMCs).

The antibodies or antigen-binding fragments thereof (e.g., bispecific antibodies) as described herein can increase immune response. In some embodiments, the antibodies or antigen-binding fragments thereof as described herein can increase immune response, activity or number of T cells (e.g., CD3+ cells, CD8+ and/or CD4+ cells) by at least 10%, 20%, 30%, 40%, 50%, 60%, 70%, 80%, 90%, 100%, 2 folds, 3 folds, 5 folds, 10 folds, or 20 folds.

In some embodiments, the antibodies or antigen-binding fragments thereof as described herein does not induce immune response in normal cells (e.g., non-tumor cells) or in the absence of tumor cells.

In some embodiments, the antibodies or antigen-binding fragments thereof as described herein are CD3 antagonist. In some embodiments, the antibodies or antigen-binding fragments thereof are CD3 agonist.

In some embodiments, the antibodies or antigen-binding fragments thereof (e.g., bispecific antibodies) can bind to CD3. Thus, the antibodies or antigen-binding fragments thereof described herein can recruit T cells to a target cell.

In some embodiments, the antibody (or antigen-binding fragments thereof) specifically binds to an antigen (e.g., a cancer antigen) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (Kd=koff/kon). In some embodiments, Kd is less than 1×10⁻⁴M, less than 1×10⁻⁵ M, less than 1×10⁻⁶ M, less than 1×10⁻⁷M, less than 1×10⁻⁸ M, less than 1×10⁻⁹ M, or less than 1×10⁻¹⁰ M. In some embodiments, the Kd is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, Kd is greater than 1×10⁴M, greater than 1×10⁻⁵ M, greater than 1×10⁻⁶ M, greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. Furthermore, Ka can be deduced from Kd by the formula Ka=1/Kd.

In some embodiments, the binding affinity to CD3 is carefully adjusted, e.g., Kd can be between 1000 nM˜10 nM, between 1000 nM˜50 nM, between 1000 nM˜100 nM, between 500 nM˜10 nM, between 500 nM˜50 nM, or between 500 nM˜100 nM.

General techniques for measuring the affinity of an antibody for an antigen include, e.g., ELISA, RIA, and surface plasmon resonance (SPR).

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI(%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen binding fragments can increase complement dependent cytotoxicity (CDC) by at least 10%, 20%, 30%, 40%, 50%, 60%, 70%, 80%, 90%, 100%, 2 folds, 3 folds, 5 folds, 10 folds, or 20 folds as compared to that of an isotype control antibody.

In some embodiments, the antibodies or antigen binding fragments can increase antibody-dependent cell-mediated cytotoxicity (ADCC) by at least 10%, 20%, 30%, 40%, 50%, 60%, 70%, 80%, 90%, 100%, 2 folds, 3 folds, 5 folds, 10 folds, or 20 folds as compared to that of an isotype control antibody.

In some embodiments, the antibodies or antigen binding fragments can increase internalization rate by at least 10%, 20%, 30%, 40%, 50%, 60%, 70%, 80%, 90%, 100%, 2 folds, 3 folds, 5 folds, 10 folds, or 20 folds as compared to that of an isotype control antibody.

In some embodiments, the antibodies or antigen binding fragments can increase phagocytosis rate by at least 10%, 20%, 30%, 40%, 50%, 60%, 70%, 80%, 90%, 100%, 2 folds, 3 folds, 5 folds, 10 folds, or 20 folds as compared to that of an isotype control antibody.

In some embodiments, the antibodies or antigen binding fragments can enhance T cell function, for example, by increasing effector T cell proliferation and/or increasing gamma interferon production by the effector T cell (e.g., as compared to proliferation and/or cytokine production prior to treatment with the antibodies or antigen binding fragments).

In some embodiments, the antibodies or antigen binding fragments enhance CD4+ effector T cell function, for example, by increasing CD4+ effector T cell proliferation and/or increasing gamma interferon production by the CD4+ effector T cell (e.g., as compared to proliferation and/or cytokine production prior to treatment with the antibodies or antigen binding fragments). In some embodiments, the cytokine is gamma interferon. In some embodiments, the antibodies or antigen binding fragments increase number of intratumoral (infiltrating) CD4+ effector T cells (e.g., total number of CD4+ effector T cells, or e.g., percentage of CD4+ cells in CD45+ cells), e.g., as compared to number of intratumoral (infiltrating) CD4+ T cells prior to treatment with antibodies or antigen binding fragments. In some embodiments, the antibodies or antigen binding fragments increase number of intratumoral (infiltrating) CD4+ effector T cells that express gamma interferon (e.g., total gamma interferon expressing CD4+ cells, or e.g., percentage of gamma interferon expressing CD4+ cells in total CD4+ cells), e.g., as compared to number of intratumoral (infiltrating) CD4+ T cells that express gamma interferon prior to treatment.

In some embodiments, the antibodies or antigen binding fragments increase number of intratumoral (infiltrating) CD8+ effector T cells (e.g., total number of CD8+ effector T cells, or e.g., percentage of CD8+ in CD45+ cells), e.g., as compared to number of intratumoral (infiltrating) CD8+T effector cells prior to treatment. In some embodiments, the antibodies or antigen binding fragments increase number of intratumoral (infiltrating) CD8+ effector T cells that express gamma interferon (e.g., percentage of CD8+ cells that express gamma interferon in total CD8+ cells), e.g., compared to number of intratumoral (infiltrating) CD8+ T cells that express gamma interferon prior to treatment with the antibody.

In some embodiments, the antibodies or antigen binding fragments enhance memory T cell function, for example by increasing memory T cell proliferation and/or increasing cytokine (e.g., gamma interferon) production by the memory cell.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibodies or antigen binding fragments can induce apoptosis.

In some embodiments, the antibodies or antigen binding fragments do not have a functional Fc region. For example, the antibodies or antigen binding fragments are Fab, Fab′, F(ab′)2, and Fv fragments.

In some embodiments, the antibodies or antigen binding fragments are humanized antibodies. Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

In some embodiments, the multi-specific antibody including the bispecific antibody described herein (e.g., a Claudin 18.2/CD3 bispecific antibody) or the trispecific antibody described herein (e.g., a Claudin 18.2/CD3 trispecific antibody) has an asymmetric structure comprising: 2, 3, 4, 5, or 6 antigen binding sites. In some embodiments, the multi-specific antibody described herein comprises 2, 3, 4, 5, or 6 antigen binding sites (e.g., antigen binding Fab domains, scFV, or naonbody (VHH)) that target a cancer antigen (e.g., Claudin 18.2). In some embodiments, the multi-specific antibody described herein comprises 2, 3, 4, 5, or 6 antigen binding sites (e.g., antigen binding Fab domains, scFV, or naonbody (VHH)) that target a T cell specific antigen (e.g., CD3). In some embodiments, the multi-specific antibody described herein (e.g., a Claudin 18.2/CD3 bispecific or trispecific antibody) comprises at least 2, 3, 4, 5, 6, or 7 common light chains. In some embodiments, the at least 2, 3, 4, 5, 6, or 7 common light chains have the same VL sequence. In some embodiments, the at least 2, 3, 4, 5, 6, or 7 common light chains have different VL sequences. In some embodiments, the cancer antigen (e.g., Claudin 18.2) binding Fab domain comprises the same VHH sequence. In some embodiments, the cancer-specific antigen (e.g., Claudin 18.2) binding Fab domain comprises different VHH sequences. In some embodiments, the C-terminus of a cancer antigen binding Fab domain is connected (e.g., covalently connected or chemically connected) to the N-terminus of a neighboring cancer antigen binding Fab domain within the same multi-specific antibody.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

In some embodiments, the antibody or antigen-binding fragment thereof described herein is a bispecific or trispecific antibody described herein. In some embodiments, the antibody or antigen-binding fragment thereof described herein binds to Claudin 18.1 or Claudin 18.1-expressing cells (e.g., CHO cells). In some embodiments, the antibody or antigen-binding fragment thereof described herein does not bind to Claudin 18.1 or Claudin 18.1-expressing cells (e.g., CHO cells). In some embodiments, the antibody or antigen-binding fragment thereof described herein binds to Claudin 18.2 or Claudin 18.2-expressing cells (e.g., CHO cells). In some embodiments, the antibody or antigen-binding fragment thereof described herein binds to Claudin 18.2 or Claudin 18.2-expressing cells (e.g., CHO cells) with a binding affinity that is at least or about 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 30 fold, 40-fold, 50-fold, or 100-fold as compared to the binding affinity to Claudin 18.1 or Claudin 18.1-expressing cells (e.g., CHO cells). In some embodiments, the binding affinities are determined by flow cytometry.

In some embodiments, the antibody or antigen-binding fragment thereof described herein has an EC50 value binding to Claudin 18.2 or Claudin 18.2-expressing cells (e.g., CHO cells) of less than or about 70 ng/ml, 60 ng/ml, 50 ng/ml, 40 ng/ml, 30 ng/ml, 20 ng/ml, 15 ng/ml, 14 ng/ml, 13 ng/ml, 12 ng/ml, 11 ng/ml, 10 ng/ml, 9 ng/ml, 8 ng/ml, 7 ng/ml, 6 ng/ml, or 5 ng/ml. In some embodiments, the antibody or antigen-binding fragment thereof described herein does not bind to cells not expressing Claudin 18.2. In some embodiments, at least or about 20%, 30%, 40%, 50%, 60%, 70%, 80%, 90%, or 95% of Claudin 18.2-expressing cells (e.g., CHO cells) bind to the antibody or antigen-binding fragment thereof described herein at 10 ng/ml after incubation at room temperature for at least or about 30 minutes, 45 minutes, 1 hour, 2 hours, or longer. In some embodiments, at least or about 70%, 75%, 80%, 85%, 90%, or 95% of Claudin 18.2-expressing cells (e.g., CHO cells) bind to the antibody or antigen-binding fragment thereof described herein at 100 ng/ml after incubation at room temperature for at least or about 30 minutes, 45 minutes, 1 hour, 2 hours, or longer.

In some embodiments, the antibody or antigen-binding fragment thereof described herein has an EC50 value binding to CD3 or CD3-expressing cells (e.g., CHO cells) of at least or about 60 μg/ml, 58 μg/ml, 55 μg/ml, 50 μg/ml, 45 μg/ml, 40 μg/ml, 39 μg/ml, 37 μg/ml, 35 μg/ml, 30 g/ml, 20 μg/ml, 10 μg/ml, 5 μg/ml, 1000 n/ml, 500 ng/ml, 400 ng/ml, or 100 ng/ml.

In some embodiments, the antibody or antigen-binding fragment thereof (e.g., bispecific antibody) described herein has a Claudin 18.2 or Claudin 18.2 expressing cell (e.g., CHO cell)-binding capability (e.g., determined by flow cytometry) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of AMG910 or AMG910 analog. In some embodiments, the antibody or antigen-binding fragment thereof described herein (e.g., bispecific antibody) has a CD3 or CD3 expressing cell (e.g., Jurkat cell)-binding capability (e.g., determined by flow cytometry) that is less than or about 50%, less than or about 40%, less than or about 30%, less than or about 25%, less than or about 20%, less than or about 15%, less than or about 10%, less than or about 5%, less than or about 4%, less than or about 3%, less than or about 2%, less than or about 1% as compared to that of AMG910 or AMG910 analog. In some embodiments, the antibody concentration is about 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 ng/ml, 5000 ng/ml, or 10000 ng/ml. Details of AMG910 can be found, e.g., in PCT/EP2019/070886, which is incorporated herein by reference.

In some embodiments, the antibody or antigen-binding fragment thereof (e.g., trispecific antibody) described herein has a Claudin 18.2 or Claudin 18.2 expressing cell (e.g., CHO cell)-binding capability (e.g., determined by flow cytometry) that is at least or about 110%, 120%, 130%, 140%, 150%, 160%, 170%, 180%, 190%, 2-fold, 3-fold, 4-fold, 5-fold, 6-fold, 7-fold, 8-fold, 9-fold, 10-fold, 20-fold, 50-fold, or 100-fold as compared to that of AMG910 or AMG910 analog. In some embodiments, the antibody or antigen-binding fragment thereof described herein (e.g., trispecific antibody) has a CD3 or CD3 expressing cell (e.g., Jurkat cell)-binding capability (e.g., determined by flow cytometry) that is less than or about 50%, less than or about 40%, less than or about 30%, less than or about 25%, less than or about 20%, less than or about 15%, less than or about 10%, less than or about 5%, less than or about 4%, less than or about 3%, less than or about 2%, less than or about 1% as compared to that of AMG910 or AMG910 analog. In some embodiments, the antibody concentration is about 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 ng/ml, 5000 ng/ml, or 10000 ng/ml.

In some embodiments, the antibody or antigen-binding fragment thereof described herein (bispecific or trispecific antibody) can induce T cell activation. In some embodiments, the T cell activation is determined by measuring percentage of CD69- and/or CD2-positive cells. In some embodiments, at least or about 10%, 15%, 20%, 25%, or 30% of T cells (e.g., PBMCs) are activated after incubating with target cells (e.g., MIA PaCa-2 cells expressing Claudin 18.2) and 10 ng/ml of the antibody or antigen-binding fragment thereof for at least or about 24 hours, 36 hours, 48 hours, 60 hours or 72 hours. In some embodiments, at least or about 25%, 30%, 35%, 40%, 45%, 50%, 55%, or 60% of T cells are activated after incubating with target cells and 100 ng/ml of the antibody or antigen-binding fragment thereof for at least or about 24 hours, 36 hours, 48 hours, 60 hours or 72 hours. In some embodiments, at least or about 40%, 45%, 50%, 55%, 60%, 65%, 70%, or 75% of T cells are activated after incubating with target cells and 1000 ng/ml of the antibody or antigen-binding fragment thereof for at least or about 24 hours, 36 hours, 48 hours, 60 hours or 72 hours. In some embodiments, the T cell activation level induced by the antibodies, or antigen-binding fragments thereof described herein is at least or about 1-fold, 2-fold, 3-fold, 4-fold, 5-fold, 6-fold, 7-fold, 8-fold, 9-fold, 10-fold, 20-fold, 50-fold, 100-fold, 500-fold, or 1000-fold as compared to that induced by an isotype control antibody (e.g., an anti-his tag antibody).

In some embodiments, the antibody or antigen-binding fragment thereof (e.g., bispecific antibody or trispecific antibody) described herein induces T cell activation that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of AMG910, AMG910 analog, or an isotype control (e.g., an anti-his tag antibody). In some embodiments, the antibody or antigen-binding fragment is purified. In some embodiments, the antibody or antigen-binding fragment is unpurified, i.e., within supernatant of antibody-producing cell culture.

In some embodiments, the antibody or antigen-binding fragment thereof described herein has an EC50 value for T cell activation of less than or about 60 ng/ml, 57 ng/ml, 55 ng/ml, 50 ng/ml, 45 ng/ml, 40 ng/ml, 35 ng/ml, 31 ng/ml, 30 ng/ml, 25 ng/ml, 24 ng/ml, 20 ng/ml, or 10 ng/ml.

In some embodiments, the antibody or antigen-binding fragment thereof described herein can induce tumor/cancer cell (e.g., MIA PaCa-2 cells expressing Claudin 18.2 cell) killing in the presence of T cells (e.g., PBMCs). In some embodiments, at least or about 60%, 65%, or 70% of target cells are killed after incubating with T cells and 1000 ng/ml of the antibody or antigen-binding fragment thereof described herein. In some embodiments, at least or about 40%, 45%, or 50% of target cells are killed after incubating with T cells and 100 ng/ml of the antibody or antigen-binding fragment thereof described herein. In some embodiments, at least or about 30%, 35%, or 40% of target cells are killed after incubating with T cells and 10 ng/ml of the antibody or antigen-binding fragment thereof described herein. In some embodiments, the cytotoxicity (i.e., the percentage of target cells being killed) of the antibody or antigen-binding fragment thereof is about or at least 1-fold, 2-fold, 3-fold, 4-fold, 5-fold, 6-fold, 7-fold, 8-fold, 9-fold, 10-fold, 20-fold, 50-fold, 100-fold, 500-fold, or 1000-fold as compared to that induced by an isotype control antibody (e.g., an anti-his tag antibody).

In some embodiments, the antibody or antigen-binding fragment thereof described herein can induce tumor/cancer cell killing and the percentage of live tumor or cancer cells are measured (e.g., by flow cytometry) after incubating with T cells (e.g., PBMCs). In some embodiments, the percentage of live cancer cells induced by the antibody or antigen-binding fragment thereof at 1 μg/ml or 0.1 μg/ml is less than or about 80%, less than or about 75%, less than or about 70%, less than or about 65%, less than or about 60%, less than or about 55%, less than or about 50%, less than or about 45%, less than or about 40%, less than or about 35%, less than or about 30% of total cancer cells. In some embodiments, the percentage of live cancer cells induced by the antibody or antigen-binding fragment thereof at 1 μg/ml or 0.1 μg/ml is less than or about 150%, less than or about 120%, less than or about 100%, less than or about 90%, less than or about 80%, less than or about 70%, less than or about 60%, less than or about 50% of that of AMG910, or AMG910 analog, or an isotype antibody control. In some embodiments, the antibody or antigen-binding fragment thereof does not deplete T cells (e.g., PBMCs).

In some embodiments, the antibody or antigen-binding fragment thereof described herein can induce cytokine (e.g., IFN-γ) release when PBMCs are mixed with target cells (e.g., Claudin18.2-expressing tumor cells). In some embodiments, the antibody or antigen-binding fragment thereof described herein does not induce cytokine (e.g., IFN-γ) release when PBMCs are not mixed with target cells (e.g., Claudin18.2-expressing tumor cells). In some embodiments, the released cytokine (e.g., IFN-γ) level induced by the antibody or antigen-binding fragment thereof is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of AMG910, AMG910 analog, or an isotype control (e.g., an anti-his tag antibody). In some embodiments, the antibody or antigen-binding fragment thereof described herein can induce complement dependent cytotoxicity (CDC) of target cells (e.g., CHO cells expressing Claudin 18.2). In some embodiments, the CDC effect induced by the antibody or antigen-binding fragment thereof is at least or about 110%, 120%, 130%, 140%, 150%, 160%, 170%, 180%, 190%, 2-fold, 3-fold, 4-fold, 5-fold, 6-fold, 7-fold, 8-fold, 9-fold, or 10-fold as compared to that of a non-specific antibody (e.g., CD20/CD3 bispecific antibody) or an isotype control antibody (e.g., serum antibodies). The “imbalanced” CD20/CD3 bispecific antibody is described, e.g., in PCT/US2018/044778, which is incorporated herein by reference.

### Antibodies and Antigen Binding Fragments

The present disclosure provides antibodies and antigen-binding fragments thereof that comprise complementary determining regions (CDRs), heavy chain variable regions, light chain variable regions, heavy chains, or light chains described herein. In some embodiments, the antibodies and antigen-binding fragments thereof are imbalanced bispecific antibodies and antigen-binding fragments thereof.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); Kontermann, R., & Dübel, S. (Eds.). (2010). Antibody engineering: Volume 2. Springer; each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding regions (Antigen binding regions: A and B), and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies or antigen-binding fragments thereof can bind to two different antigens or two different epitopes. In some embodiments, the antibodies or antigen-binding fragments thereof can bind to three different antigens or three different epitopes.

An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

In some embodiments, the scFv described herein comprises from N-terminus to C-terminus: VH; the polypeptide linker; and VL. In some embodiments, the scFv described herein comprises from N-terminus to C-terminus: VL; the polypeptide linker; and VH. In some embodiments, the linker peptide comprises a sequence that is at least 80%, 85%, 90%, 95%, or 100% identical to any one SEQ ID NOs: 35-40. In some embodiments, the linker peptide comprises a sequence that is at least or about 80%, 85%, 90%, 95%, or 100% identical to SEQ ID NO: 36 or 37. In some embodiments, the linker peptide comprises a sequence that is at least or about 80%, 85%, 90%, 95%, or 100% identical to one or more (e.g., 1, 2, 3, 4, 5, 6, 7, or 8) repeats of GGGGS (SEQ ID NO: 35).

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Linear antibodies comprise a pair of tandem Fd segments (VH-CH1-VH-CH1) which, together with complementary light chain polypeptides, form a pair of antigen binding regions. Linear antibodies can be bispecific or monospecific.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′2 homodimers through digestion with pepsin. Another way to form antibody homodimers is through the use of the autophilic T15 peptide described in Zhao et al. (J. Immunol. 25:396-404, 2002).

Antibodies and antibody fragments of the present disclosure can be modified in the Fc region to provide desired effector functions or serum half-life.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments (e.g., bispecific antibodies) described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the multispecific antibody or antigen-binding fragment thereof described herein increases T cell activation (e.g., as indicated by percentage of CD69+CD2+ cells, the percentage of CD69+ cells, or specific killing of target cells) by at least or about 10%, 20%, 30%, 40%, 50%, 60%, 70%, 80%, 90%, 100%, 2 folds, 3 folds, 4 folds, 5 folds, 6 folds, 7 folds, 8 folds, 9 folds, 10 folds, 20 folds, 30 folds, 40 folds, 50 folds, 100 folds, or more, as compared to an isotype control antibody, or a monoclonal antibody comprising the same VH or VL of the bispecific antibody. In some embodiments, the T cells are selected from Jurkat cells or PBMCs. In some embodiments, the target cells are selected from pancreatic cancer cells (e.g., MIA PaCa-2 cells) or gastric cancer cells. In some embodiments, the target cells expresses cancer antigens (e.g., Claudin 18.2) endogenously or by transient transfection. In some embodiments, the T cells activation is mediated by the multi-specific antibody or antigen-binding fragment described herein at a concentration that is less than or about 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 700 ng/ml, 800 ng/ml, 900 ng/ml, or 1000 ng/ml. In some embodiments, the T cells, target cells, and the multi-specific antibody are incubated for at least or about 8 hours, 16 hours, 24 hours, 36 hours, 48 hours, 60 hours, or 72 hours. In some embodiments, the ratio of the T cells and target cells is about 1:1, about 2:1, about 3:1, about 5:1, about 10:1, about 20:1, or about 25:1.

In some embodiments, the multi-specific antibody or antigen-binding fragment thereof described herein (e.g., a Claudin 18.2/CD3 bispecific or trispecific antibody) binds to an antigen (e.g., Claudin 18.2) with a binding affinity that is about 50%, about 60%, about 70%, about 80%, about 90%, about 100%, about 110%, about 120%, about 130%, about 140%, about 150%, or about 200% to that of a heavy-chain antibody (e.g., an anti-Claudin 18.2 heavy-chain antibody) comprising the same VHH of the multi-specific antibody.

In some embodiments, the multispecific antibody or antigen-binding fragment thereof described herein (e.g., a Claudin 18.2/CD3 bispecific or a Claudin 18.2/CD3 trispecific antibody) mediates complement-dependent cytotoxicity (CDC) to at least or about 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 30-fold, 40-fold, or 50-fold as compared to that mediated by an isotype control antibody.

In some embodiments, the multi-specific antibody or antigen-binding fragment thereof described herein (e.g., a Claudin 18.2/CD3 bispecific or trispecific antibody) is internalized at a percentage that is about 50%, about 60%, about 70%, about 80%, about 90%, about 100%, about 110%, about 120%, about 130%, about 140%, about 150%, or about 200% to that of a monoclonal antibody (e.g., an anti-Claudin 18.2 antibody, an anti-CD3 antibody, or an isotype antibody control).

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

### Methods of Making Antibodies

An isolated fragment of human protein (e.g., Claudin 18.2, CD3, or cancer antigens) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDR1, 2 and 3, generated by overlapping PCR, to a modified human VH scaffold to generate enhanced diversity and keep low immunogenicity. The VHH libraries can be then panned against antigens to obtain VHH with desired binding affinities.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, one or more amino acid residues in the CH3 portion of the IgG are substituted. In some embodiments, one heavy chain has one or more of the following substitutions Y349C and T366W. The other heavy chain can have one or more the following substitutions E356C, T366S, L368A, and Y407V. Furthermore, a substitution (-ppcpScp->-ppcpPcp-) can also be introduced at the hinge regions of both substituted IgG. In some embodiments, one heavy chain has a T366Y (knob) substitution, and the other heavy chain has a Y407T (hole) substitution (EU numbering).

One aspect of the present application provides a heteromultimeric (e.g., heterodimeric) protein comprising a first polypeptide comprising a first heavy chain constant domain 3 (CH3) domain and a second polypeptide comprising a second CH3 domain, wherein the first CH3 domain comprises a substitution relative to a wildtype CH3 domain at amino acid position 354 with a bulky hydrophobic amino acid, and/or the second CH3 domain comprises a substitution relative to a wildtype CH3 domain at amino acid position 347 with a negatively charged amino acid, and wherein the amino acid residue numbering is based on EU numbering. In some embodiments, the bulky hydrophobic amino acid at amino acid position 354 forms a hydrophobic interaction with an amino acid residue in the second CH3 domain. In some embodiments, the second CH3 domain comprises a bulky hydrophobic residue at amino acid position 349 (e.g., Y349). In some embodiments, the negatively charged amino acid at amino acid position 347 forms an ionic bond with an amino acid residue in the first CH3 domain. In some embodiments, the first CH3 domain comprises a positively charged residue at amino acid position 360 (e.g., K360). In some embodiments, the first CH3 domain and the second CH3 domain are human CH3 domains. In some embodiments, the first CH3 domain comprises a substitution selected from the group consisting of S354Y, S354F and S354W. In some embodiments, the first CH3 domain comprises S354Y. In some embodiments, the second CH3 domain does not comprise a compensatory substitution (e.g., a substitution at Y349) for the substitution of S354 in the first CH3 domain. In some embodiments, the second CH3 domain comprises a substitution selected from the group consisting of Q347E and Q347D. In some embodiments, the second CH3 domain comprises Q347E. In some embodiments according to any one of the heteromultimeric proteins described above, the first CH3 domain and the second CH3 domain further comprise knob-into-hole (KIH) residues. In some embodiments, the knob-into-hole residues are T366Y and Y407T. In some embodiments, the first CH3 domain comprises T366Y and S354Y, and the second CH3 domain comprises Y407T and Q347E. In some embodiments, the first CH3 domain comprises Y407T and S354Y, and the second CH3 domain comprises T366Y and Q347E. Details can be found, e.g., in PCT/US2020/025469, which is incorporated herein by reference.

Furthermore, an anion-exchange chromatography can be used to purify bispecific antibodies. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific antibody having Arm A and Arm B will be somewhere between the two pIs of the homodimers. Thus, the two homodimers and the bispecific antibody can be released at different pH conditions. The present disclosure shows that a few amino acid residue substitutions can be introduced to the heavy chains to adjust pI.

Thus, in some embodiments, the amino acid residue at Kabat numbering position 83 is lysine, arginine, or histidine. In some embodiments, the amino acid residues at one or more of the positions 1, 6, 43, 81, and 105 (Kabat numbering) is aspartic acid or glutamic acid.

In some embodiments, the amino acid residues at one or more of the positions 13 and 105 (Kabat numbering) is aspartic acid or glutamic acid. In some embodiments, the amino acid residues at one or more of the positions 13 and 42 (Kabat numbering) is lysine, arginine, histidine, or glycine.

Bispecific antibodies can also include e.g., cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bispecific antibodies from antibody fragments are also known in the art. For example, bispecific antibodies can be prepared using chemical linkage. Brennan et al. (Science 229:81, 1985) describes a procedure where intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bispecific antibody.

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody fragments, antibody variants, antibody constructs or binding domains. Random mutations inside the CDRs are introduced using radiation, chemical mutagens or error-prone PCR. In addition, the genetic diversity can be increased by chain shuffling. Two or three rounds of mutation and selection using display methods like phage display usually results in antibodies, antibody fragments, antibody variants, antibody constructs or binding domains with affinities in the low nanomolar range.

### Methods of Treatment

The methods described herein include methods for the treatment of disorders associated with cancer. Generally, the methods include administering a therapeutically effective amount of engineered bispecific antibodies (e.g., imbalanced bispecific antibodies) of antigen-binding fragments thereof as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein (e.g., imbalanced bispecific antibodies) for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof, or an antibody drug conjugate disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is a cancer expressing Claudin 18.2.

In some embodiments, the cancer is a solid tumor. In some embodiments, the cancer is gastric or pancreatic adenocarcinoma. In some embodiments, the cancer is breast cancer, biliary tract cancer, gastric cancer, hepatocellular carcinoma, renal cell carcinoma, pancreatic cancer, non-small cell lung cancer, esophageal cancer, mucinous ovarian cancer, or mesothelioma.

In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of antibody used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, antigen-binding fragment, antibody-drug conjugates, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Also provided herein are pharmaceutical compositions that contain at least one (e.g., one, two, three, or four) of the antibodies, antigen-binding fragments, or antibody-drug conjugates described herein. Two or more (e.g., two, three, or four) of any of the antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Example 1. Materials and Methods

Jurkat (Clone E6-1ATCC® TIB-152™), PANC-1 (ATCC® CRL-1469™) and MIA PaCa-2 (ATCC® CRL-1420™) cancer cells were obtained from American Type Culture Collection (ATCC). MIA PaCa-2 stable cell clone expressing full-length human Claudin 18.2 receptor-MIA PaCa-2+ Claudin 18.2 (Cat #C3002) was purchased from Accurus Biosciences. Human peripheral blood mononuclear cells (PBMCs) were purchased from HumanCells Biosciences. All cells were maintained in RPMI 1640 media (Gibco) supplemented with 10% fetal bovine serum at 37° C. and 5% CO₂.

The lead candidates were discovered as follows. First, a phage VHH library was used for panning for Claudin 18.2 binders. Specifically, Claudin 18.2 recombinant protein was used for the first two rounds of panning. For the third round of panning, Claudin 18.2-expressing cells were used and counter panning was carried out against Claudin 18.1-expressing cells. Meanwhile, Claudiximab competitive binders were also screened by panning. Claudin 18.2 binders were screened and characterized. Specifically, candidates were screened to isolate those binding to Claudin 18.2-expressing cells but not Claudin 18.1-expressing cells. The candidates were also tested for cross-binding activities to mouse, monkey, and human Claudin 18.2. Claudiximab was also used for competitive binding verification. The receptor binding and T cell activation activities were then verified by cell activation assays.

Claudin 18.2/CD3 bispecific antibodies (BsAbs) were constructed and characterized from these binders. Specifically, affinity maturation of Claudin 18.2 binding arm can be performed. Receptor binding, T cell activation, and/or cancer cell killing assays were performed. Lead sequences were optimized and produced. This step also included structural optimization and lead characterization. BsAb functions were verified using cell based assays and/or animal studies. The cell based assays included receptor binding, T cell activation, cancer cell killing, ADCC, and/or CDC assays.

### Example 2. Screening for Claudin 18.2 Binding Lead Candidate

To screen the library for Claudin 18.2 binding antibodies, Claudin 18.1 and Claudin 18.2 expressing cells were used. 5×10⁴ cells/well of CHO, CHO+ Claudin 18.1 and CHO+ Claudin 18.2 cells in 100 μl FACS buffer (1×PBS with 2% FBS) were seeded in a V-bottom 96-well plate. The cells were then treated with 100 μl of unpurified cell culture supernatants containing Claudin 18.2-targeting antibodies. After incubating the plate at room temperature (RT) for 45 minutes, the cells were washed twice with the FACS buffer. The cell pellets were resuspended in 100 μl FACS buffer containing anti-c-Myc antibody 9E10 (1:500). Afterwards, the plate was incubated at RT for 30 minutes and then washed twice with the FACS buffer. After washing, cell pellets were resuspended in 100 μl FACS buffer containing Avidin-PE (1:200), and the plate was incubated at RT for 30 minutes. After the incubation, the cells were washed twice with the FACS buffer. Cell pellets were then resuspended in 120 μl FACS buffer, and the percentage of PE-positive cells were analyzed by flow cytometry.

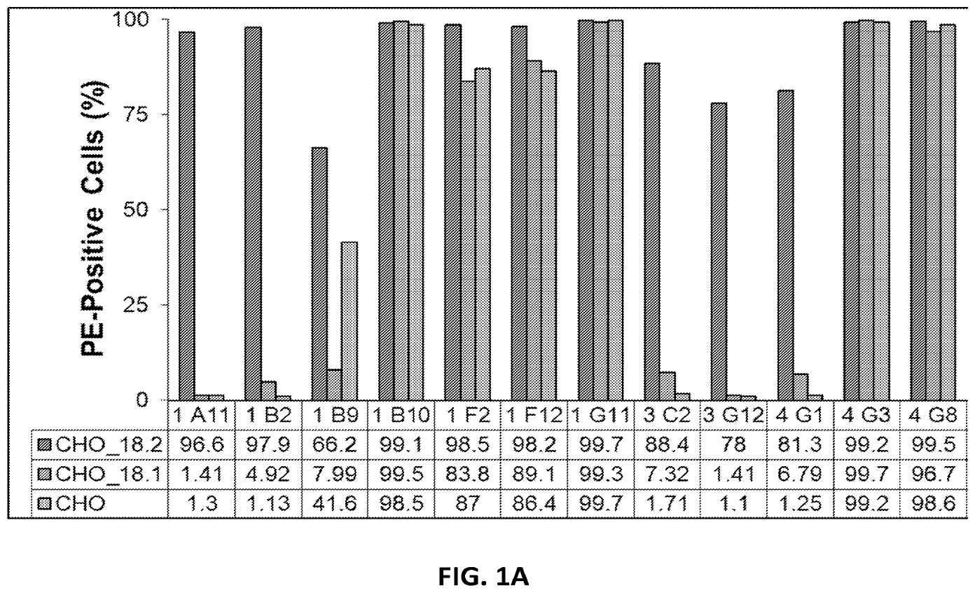

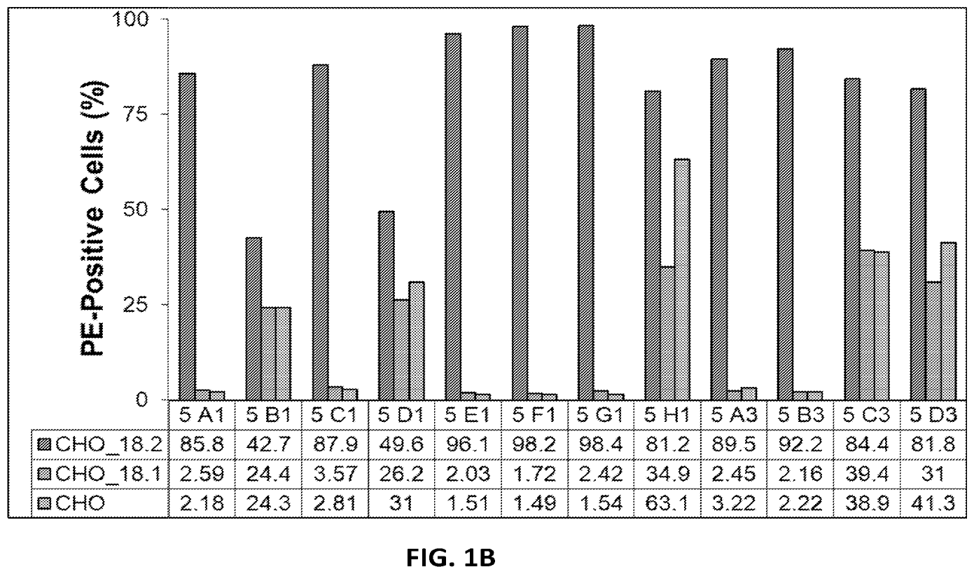

Specifically, for screening the lead candidates that binds to Claudin 18.2 but not Claudin 18.1, CHO cells expressing Claudin 18.1 (CHO+ Claudin 18.1) and Claudin 18.2 (CHO+ Claudin 18.2) were generated. After treating CHO+ Claudin 18.2 cells with 384 Claudin 18.2-targeting antibody candidates screened from a phage library, 10% of the candidates were shown to bind to Claudin 18.2 expressing cells. These candidates were further tested for Claudin 18.1 binding using the CHO+ Claudin 18.1 cells. Among the 384 candidates, Claudin 18.2-1A11, Claudin 18.2-1B2, Claudin 18.2-3C2, and Claudin 18.2-4G1 showed high binding affinities for Claudin 18.2, with minimal affinity for Claudin 18.1 (FIGS. 1A-1B). These lead clones were used to generate anti-Claudin 18.2/CD3 bispecific antibodies (BsAbs). The sequences were also further humanized. The humanized sequences are shown in FIG. 22.

### Example 3. Screening of Claudin 18.2/CD3 BsAb Lead Candidate

To compare the half maximal effective concentration (EC50) of the lead BsAbs, 8.5×10⁴ cells/well of CHO+ Claudin 18.2 cells in 100 μl FACS buffer were seeded in a V-bottom 96-well plate. The cells were then treated with 100 μl of unpurified cell culture supernatants containing Claudin 18.2-1A11/CD3, Claudin 18.2-1B2/CD3, Claudin 18.2-3C2/CD3, and Claudin 18.2-4G1/CD3 BsAbs with the BiSpecific-V1 structure as shown in FIG. 6A. The final concentrations of the BsAbs were 10 μg/ml, 1 μg/ml, 100 ng/ml, 10 ng/ml, 1 ng/ml, 100 pg/ml, 10 pg/ml, and 1 pg/ml. After incubating the plate at RT for 45 minutes, the cells were washed twice with the FACS buffer and cell pellets were resuspended in 100 μl FACS buffer containing goat anti-human-FITC (1:200) for 30 minutes at RT in dark. Afterwards, the cells were washed twice with FACS buffer, and cell pellets were resuspended in 120 μl FACS buffer. The percentage of FITC-positive cells were analyzed by flow cytometry.

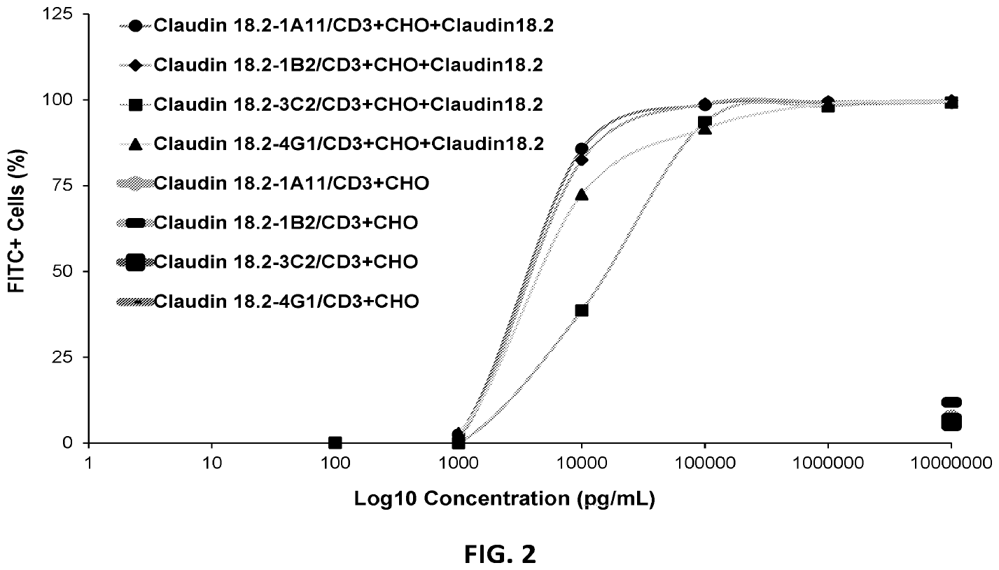

Specifically, Claudin 18.2-1A11/CD3, Claudin 18.2-1B2/CD3, Claudin 18.2-3C2/CD3, and Claudin 18.2-4G1/CD3 T-cell targeting BsAbs were designed, and their EC50 values were determined using CHO+ Claudin 18.2 cells. As shown in FIG. 2, Claudin 18.2-1A11/CD3 and Claudin 18.2-1B2/CD3 showed strongest binding to CHO+ Claudin 18.2 cells as compared to others, both with EC50 values of about 5 ng/ml. By contrast, Claudin 18.2-3C2/CD3 showed the weakest binding among the four lead BsAbs, with an EC50 value of about 13 ng/mL. In addition, the EC50 value of Claudin 18.2-4G1/CD3 was determined as about 6 ng/ml. Because Claudin 18.2-1A11/CD3 and Claudin 18.2-1B2/CD3 showed similar binding affinity against Claudin 18.2, Claudin 18.2-1A11/CD3 was selected for subsequence experiments. Further, none of the lead candidates showed any binding to control CHO cells (not expressing Claudin 18.2). The results indicate that these BsAbs have a low risk of off-target binding and are generally safe.

### Example 4. Induction of T Cell Activation by Claudin 18.2-1A11/CD3 BsAb

For T cell activation assays, 1×10⁴ cells/well of MIA PaCa-2+ Claudin 18.2 cells (MIA PaCa-2 cells overexpressing Claudin 18.2) were plated in 100 μl RPMI 1640 media supplemented with 10% FBS in a flat-bottom 96-well plate. After culturing the cells overnight at 37° C. and 5% CO₂, the cells were treated with 50 μl of purified Claudin 18.2-1A11/CD3 antibody (diluted to final concentrations of 1 μg/ml, 100 ng/ml, 10 ng/ml, and 1 ng/ml), followed by 1×10⁵ cells/well of 5 donor pooled PBMCs in 50 μl RPMI 1640 media supplemented with 10% FBS. The plate was incubated at 37° C. and 5% CO₂ for 48 hours, and then the media containing PBMCs were transferred to a V-bottom 96-well plate. Afterwards, the PBMCs were washed twice with FACS buffer. The cell pellets were resuspended in 100 μl FACS buffer containing a PE-conjugated CD69 antibody (CD69-PE; diluted at 1:100) and an APC-conjugated CD2 antibody (CD2-APC; diluted at 1:100), followed by incubation for 30 minutes at RT in dark. After the incubation, the cell pellets were washed twice with the FACS buffer, and then resuspended in 120 μl FACS buffer. The percentage of CD69+ and CD2+ cells were analyzed by flow cytometry.

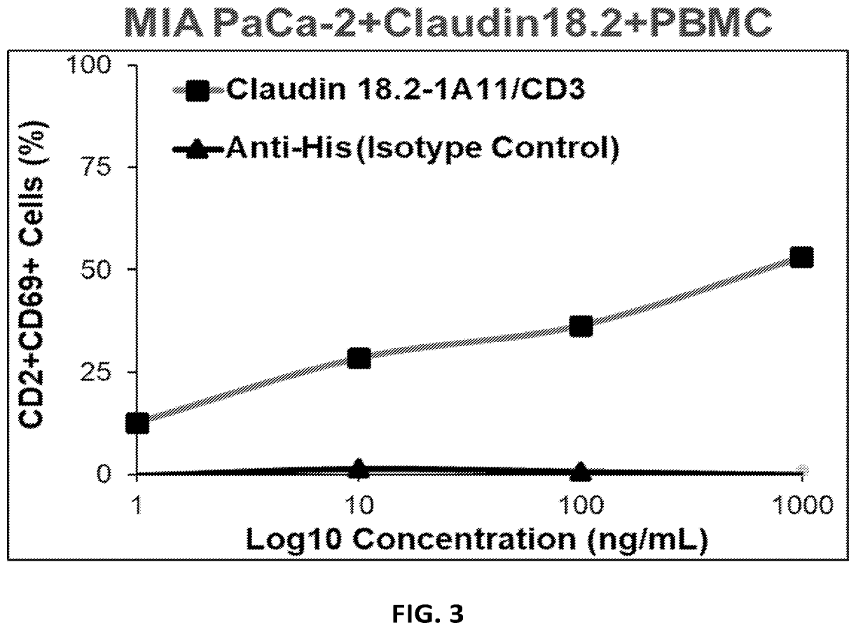

Specifically, for evaluating T cell activation by Claudin 18.2-1A11/CD3, human pooled PBMC from 5 donor was co-cultured with Claudin 18.2-overexpressing MIA PaCa-2 cells in the presence of the antibodies. As shown in FIG. 3, Claudin 18.2-1A11/CD3 showed a concentration-dependent increase of T cell activation. By contrast, T cell activation was not observed using an isotype control antibody.

### Example 5. Induction of Tumor Cell Killing (TCK) by Claudin 18.2-1A11/CD3 BsAb

For Tumor cell killing (TCK), 1×10⁴ cells/well of MIA PaCa-2+ Claudin 18.2 cells (MIA PaCa-2 cells overexpressing Claudin 18.2) were plated in 100 μl RPMI 1640 media supplemented with 10% FBS media in a flat-bottom 96-well plate. After culturing the cells overnight at 37° C. and 5% CO₂, the cells were treated with 50 μl of purified Claudin 18.2-1A11/CD3 antibody (diluted to final concentrations of 1 pg/ml, 100 ng/ml, 10 ng/ml, and 1 ng/ml), followed by 1×10⁵ cells/well of 5 donor pooled PBMCs in 50 μl RPMI 1640 media supplemented with 10% FBS. The plate was incubated at 37° C. and 5% CO₂ for 48 hours, and then the supernatant containing PBMCs was discarded. The cancer cell cytotoxicity was analyzed using crystal violet staining following manufacturer's protocol (Bio Vision Inc.; Cat #K329).

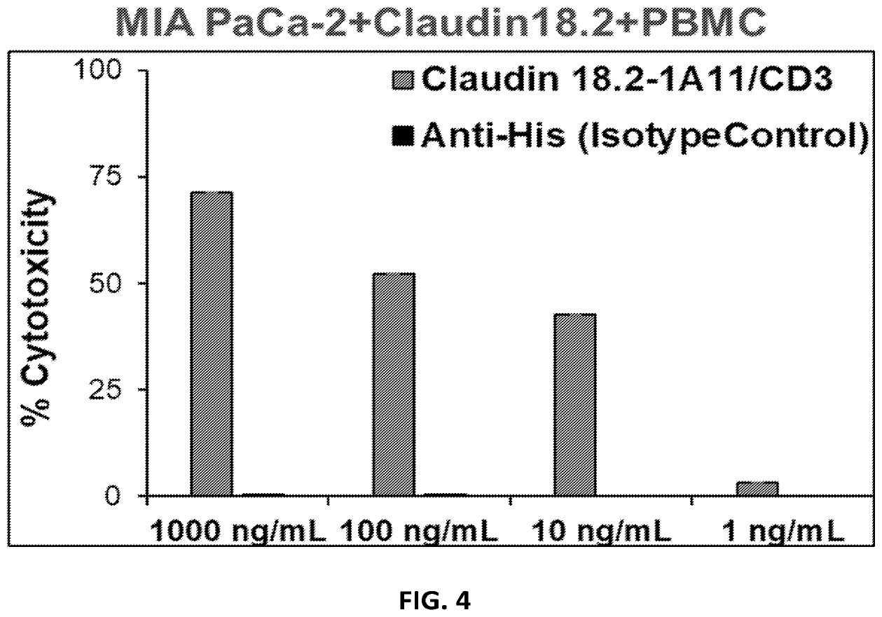

Specifically, for evaluating tumor cell killing (TCK) by Claudin 18.2-1A11/CD3, human pooled PBMC from 5 donor was co-cultured with Claudin 18.2 overexpressing MIA PaCa-2 cells in presence of the antibodies. As shown in FIG. 4, Claudin 18.2-1A11/CD3 induced a concentration-dependent increase in TCK. By contrast, no cancer cell cytotoxicity was observed using an isotype control antibody.

### Example 6. Induction of Complement Dependent Cytotoxicity (CDC) by Claudin 18.2-1A11/CD3

2×10⁴ cells/well of CHO+ Claudin 18.2 and CHO cells were plated in 100 μl RPMI 1640 (without FBS) media in a U-bottom 96-well plate. After incubating the plate at 37° C. and 5% CO₂ for 10 minutes, the cells were treated with 10 pg/ml purified Claudin 18.2-1A1l1/CD3 or CD20/CD3 bispecific antibodies in 50 ul RPMI 1640 (without FBS) media and mixed gently. After 15-20 minutes of incubation at 37° C. and 5% C02, 10% pooled human complement serum (ICSER25ML-31708) was added to the cells in 50 ul RPMI 1640 (without FBS). The plate was then incubated at 37° C. and 5% CO₂ for 4 hours. After the incubation, complement dependent cytotoxicity (CDC) was analyzed using CyQUANT™ LDH Cytotoxicity Assay kit following manufacturer's protocol (Invitrogen, Cat #C20301).

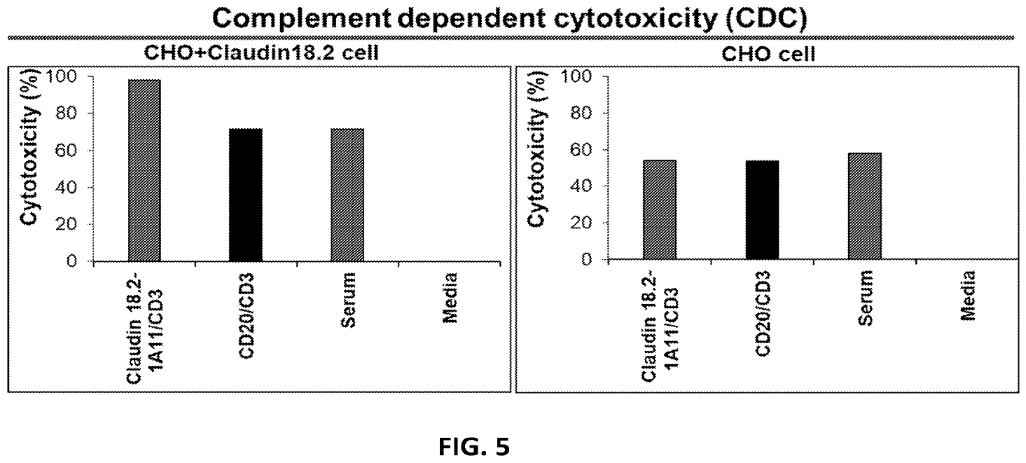

Specifically, for evaluating the induction of complement dependent cytotoxicity (CDC) by Claudin 18.2-1A11/CD3, Claudin 18.2 overexpressing CHO cell was treated with 18.2-1A11/CD3 in presence of 10% pooled human complement serum. As shown in FIG. 5, no significant difference in the inductions of CDC by Claudin 18.2-1A11/CD3, CD20/CD3 control antibody and serum were observed against CHO cells. However, CDC by Claudin 18.2-1A11/CD3 was significantly enhanced compared to the CD20/CD3 control antibody and serum against Claudin 18.2-overexpressing cells.

### Example 7. Cell Binding Assays by Claudin 18.2/CD3 Bispecific or Trispecific Antibodies

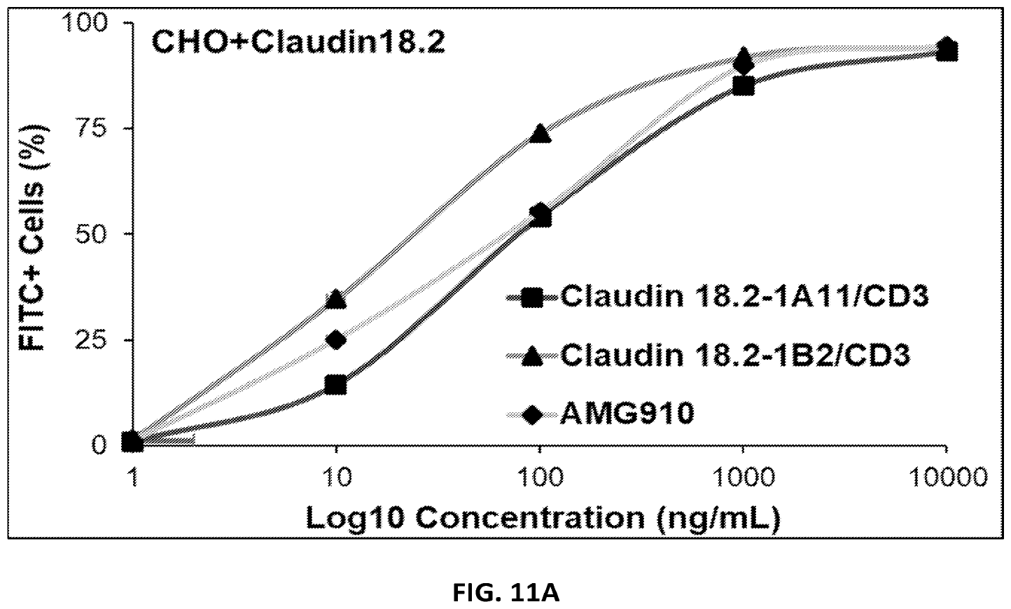

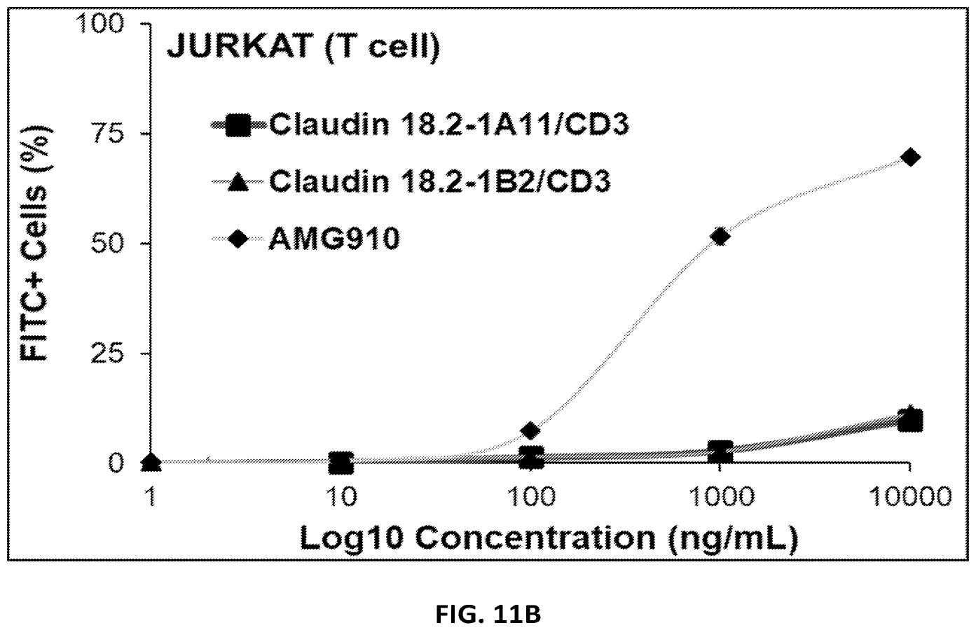

Binding capabilities of the lead bispecific antibodies (BsAbs) Claudin 18.2-1A11/CD3 and Claudin 18.2-1B2/CD3 to Claudin 18.2 or CD3 were determined, using Claudin 18.2-expressing CHO cells (CHO+ Claudin18.2) or Jurkat cells, respectively. The experiment was carried out as described in Example 3, except that the antibodies were diluted to final concentrations of 1 ng/ml, 10 ng/ml, 100 ng/ml, 1000 ng/ml and 10000 ng/ml. AMG910 analog was used as a control antibody. A schematic structure of Claudin 18.2-1A11/CD3 and Claudin 18.2-1B2/CD3 BsAbs is shown in FIG. 6A with the BiSpecific-V1 structure. As shown in FIGS. 11A-11B, the Claudin 18.2/CD3 BsAbs bound to CHO+ Claudin18.2 cells similar to that of AMG910 analog. However, the Claudin 18.2/CD3 BsAbs exhibited much weaker binding capabilities to CD3+ Jurkat cells as compared to that of AMG910 analog. The poor binding to CD3 may render the Claudin 18.2/CD3 BsAbs a higher safety as compared to that of AMG910 analog.

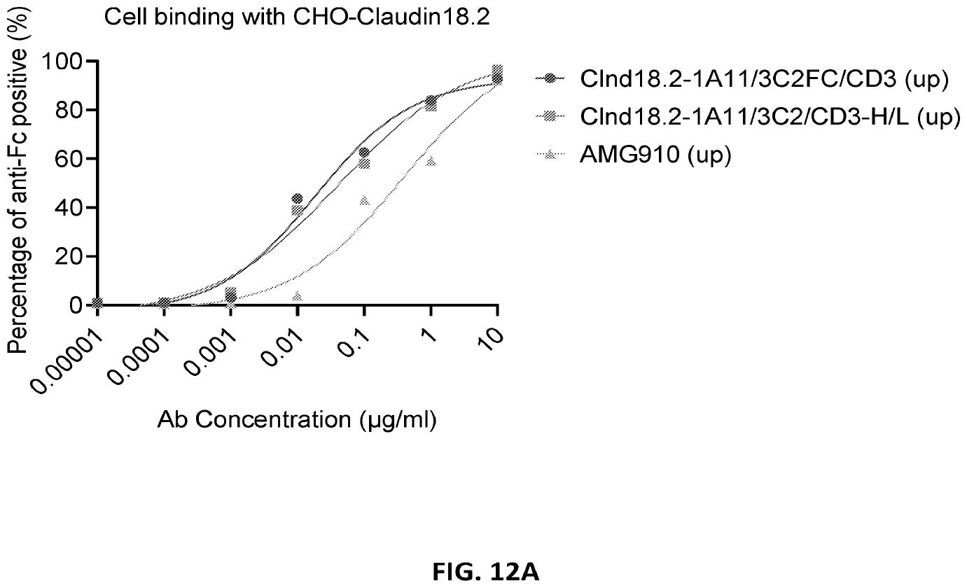

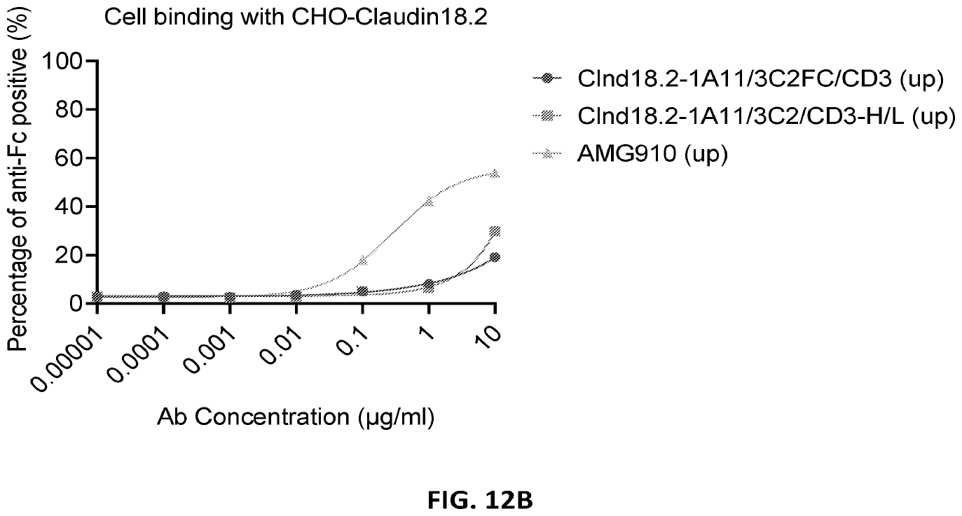

Similarly, binding capabilities of the lead Claudin 18.2-1A11/3C2/CD3 trispecific antibodies (TsAbs) to Claudin 18.2 or CD3 were determined, using Claudin 18.2-expressing CHO cells (CHO+ Claudin18.2) or Jurkat cells, respectively. The experiment was carried out as described in Example 3, except that the antibodies were diluted to final concentrations of 0.1 ng/ml, 1 ng/ml, 10 ng/ml, 100 ng/ml, 1000 ng/ml and 10000 ng/ml. A schematic structure of Clnd18.2-1A11/3C2FC/CD3(up) is shown in FIG. 6B with the TriSpecific-V1 structure. A schematic structure of Clnd18.2-1A11/3C2/CD3-H/L(up) is shown in FIG. 9B with the TriSpecific-V4 structure. As shown in FIGS. 12A-12B, the Claudin 18.2/CD3 TsAbs bound to CHO+ Claudin18.2 cells with a higher binding capabilities than that of AMG910 analog. However, the Claudin 18.2/CD3 TsAbs exhibited much weaker binding capabilities to CD3+ Jurkat cells as compared to that of AMG910 analog. The poor binding to CD3 may render the Claudin 18.2/CD3 TsAbs a higher safety as compared to that of AMG910 analog.

### Example 8. Induction of T Cell Activation by Claudin 18.2/CD3 Multispecific Antibodies

T cells activation assays were carried out as described in Example 4. Specifically, Claudin 18.2-1B2/CD3 BsAb (with the BiSpecific-V1 structure as shown in FIG. 6A), Claudin 18.2-1A11/3C2FC/CD3 TsAb (with the TriSpecific-V1 structure as shown in FIG. 6B), and Claudin 18.2-1B2/3C2FC/CD3 TsAb (with the TriSpecific-V1 structure as shown in FIG. 6B) were diluted to final concentrations of 1 ng/ml, 10 ng/ml, 100 ng/ml, 1000 ng/ml, and 10000 ng/ml. The antibodies were incubated with Claudin 18.2-overexpssing MIA PaCa-2 cells and PBMCs. The percentage of CD69-positive cells were analyzed by flow cytometry. Either purified antibodies or unpurified antibody (supernatant of cell culture) were used.

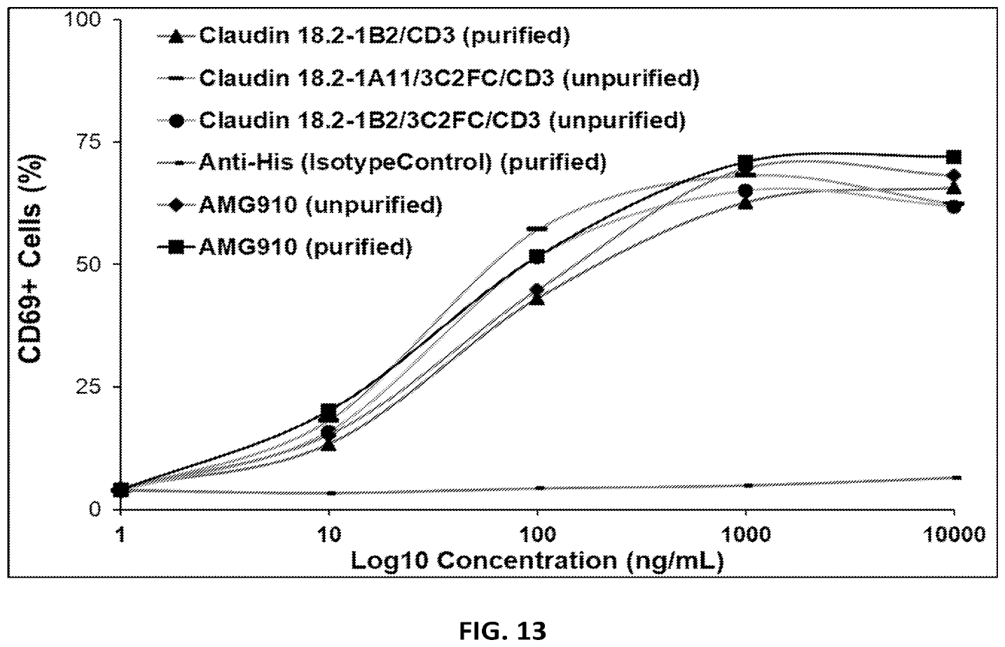

As shown in FIG. 13 and the table below, the Claudin 18.2/CD3 multispecific antibodies induced comparable T cell activation as compared to that of AMG910 analog. In particular, the Claudin 18.2-1A 11/3C2FC/CD3 and Claudin 18.2-1B2/3C2FC/CD3 trispecific antibodies stimulated T cell activation with lower EC50 values as compared to that of the Claudin 18.2-1B2/CD3 BsAb or AMG910 analog.

TABLE 1

| | Claudin 18.2- | Claudin 18.2- | Claudin 18.2- | AMG910 | AMG910 |
|---|---|---|---|---|---|
| Antibody | 1B2/CD3 | 1A11/3C2FC/CD3 | 1B2/3C2FC/CD3 | analog (Sup) | analog |
| EC50 (ng/ml) | 56.05 | 23.55 | 30.36 | 57.71 | 34.54 |

### Example 9. Induction of Tumor Cell Killing (TCK) by Claudin 18.2/CD3 Trispecific Antibodies

Tumor cell killing (TCK) assays were carried out as described in Example 5. Specifically, Clnd18.2/CD3-V3-3C2/1A1l1(up) TsAb (with the TriSpecific-V3 structure as shown in FIG. 8B) and Clnd18.2-1A11/3C2/CD3-H/L(up) TsAb (with the TriSpecific-V4 structure as shown in FIG. 9B) were diluted to final concentrations of 0.1 μg/ml and 1 μg/ml. The antibodies were incubated with Claudin 18.2-overexpssing MIA PaCa-2 cells and PBMCs. The percentage of live Claudin 18.2-overexpssing MIA PaCa-2 cells was analyzed after the incubation. AMG910 analog was used as a control.

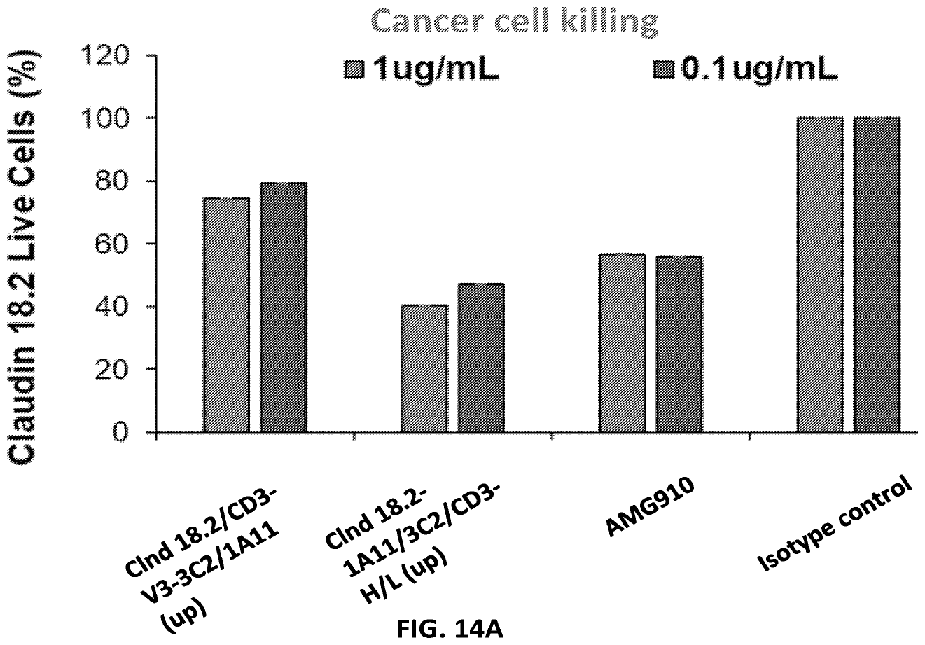

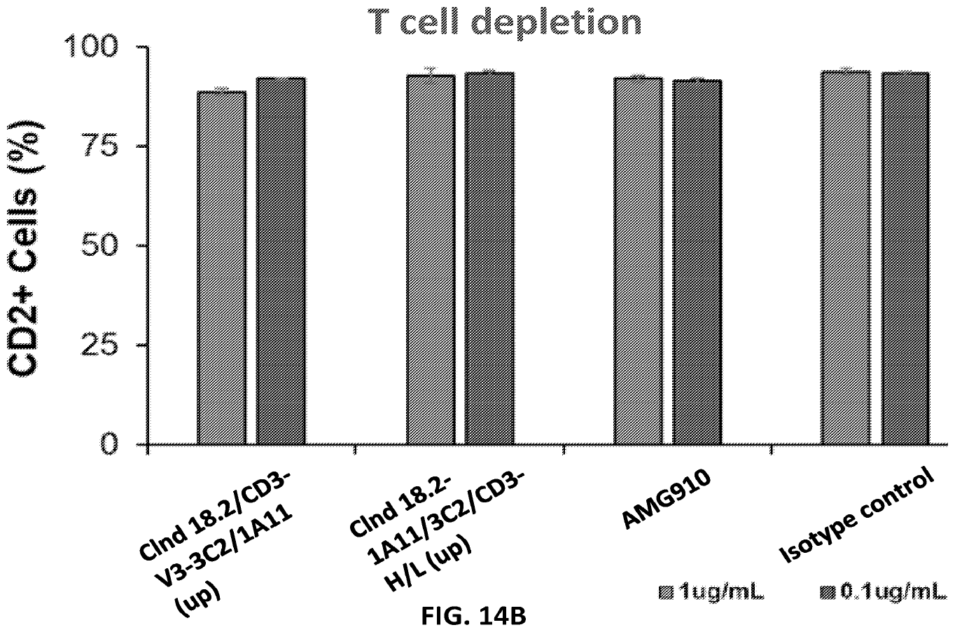

As shown in FIG. 14A, the Claudin 18.2/CD3 trispecific antibody Clnd18.2-1A11/3C2/CD3-H/L(up) had better cancer cell killing efficiency as compared to that of the Clnd18.2/CD3-V3-3C2/1A11(up) or AMG910 analog.

T cell depletion was also determined by analyzing the percentage of CD2+ cells. As shown in FIG. 14B, none of the tested antibodies exhibited any T cell depletion activity.

### Example 10. Summary

The table below summarized the results as shown early examples. N/A is not applicable or not tested. “−” is no activity. “+” is low activity. “+++” is medium activity. “+++++” is high activity. The BiSpecific-V1 format results are based on the experimental data from Claudin 18.2-1B2/CD3 BsAb described herein. The TriSpecific-V1 format results are based on the experimental data from Claudin 18.2-1A11/3C2FC/CD3 TsAb described herein. The TriSpecific-V4 format results are based on the experimental data from Clnd18.2-1A11/3C2/CD3-H/L(up) TsAb described herein.

TABLE 2

| | Claudin | CD3 | | | Tumor cell |
|---|---|---|---|---|---|
| | 18.2 | binding | T cell | T cell | killing |
| Antibody | binding | (T cell) | activation | activation | MIA PaCa + |
| Formats | EC50 | EC50 | EC50 | CHO only | Claudin 18.2 |
| BiSpecific-V1 | 67 | 58890 | 56 | − | +++ |
| | ng/mL | ng/mL | ng/mL | | |
| TriSpecific-V1 | 15 | 37000 | 23.5 | − | N/A |
| | ng/mL | ng/mL | ng/mL | | |
| TriSpecific-V4 | 33 | 39000 | ~34.5 | N/A | +++++ |
| | ng/mL | ng/mL | ng/mL | | |
| AMG 910 | 47 | 485 | 34.5 | + | +++ |
| | ng/mL | ng/mL | ng/mL | | |

### Example 11. Determination of the Effect of Claudin 18.2-1B2/CD3 BsAb on Cancer Cell Killing and T Cell Activation

Pre-labeled Claudin18.2-overexpressing MIA PaCa cells were mixed with PBMCs from healthy donors (donors 1-6) at a 1:5 ratio and plated in a 96-well flat-bottom plate (5×10⁴ cells/well). The cell mixture was treated with Claudin 18.2-1B2/CD3 BsAb (with the BiSpecific-V4 structure as shown in FIG. 9A) at indicated concentrations, and then co-cultured in a CO₂ incubator for 72 hours. Afterwards, the cells were collected, and supernatant was collected for IFN-γ analysis by ELISA. The attached cells were digested with Accutase® and washed twice with FACS buffer. After washing, the cells were resuspended in 60 μl of FACS buffer containing CD2-APC (1:500 dilution), CD69-PE (1:100 dilution), and CD25-APC-Cy7 (1:500 dilution), followed by a 30-minute incubation at room temperature. After the incubation, the cells were washed twice with FACS buffer and resuspended in 80 μl FACS buffer containing 7-AAD (1:100). After 5 minutes at room temperature, the cells were analyzed by FACS using a NovoCyte® cytometer.

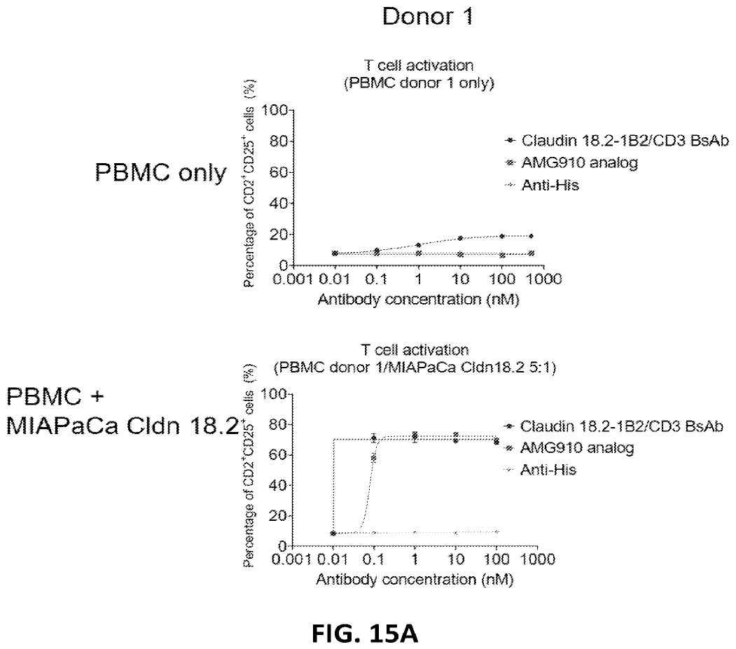

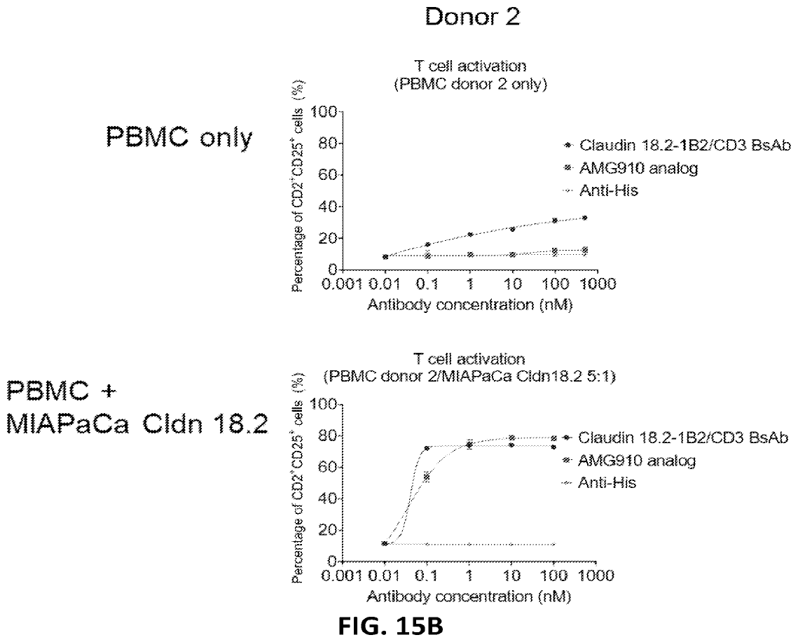

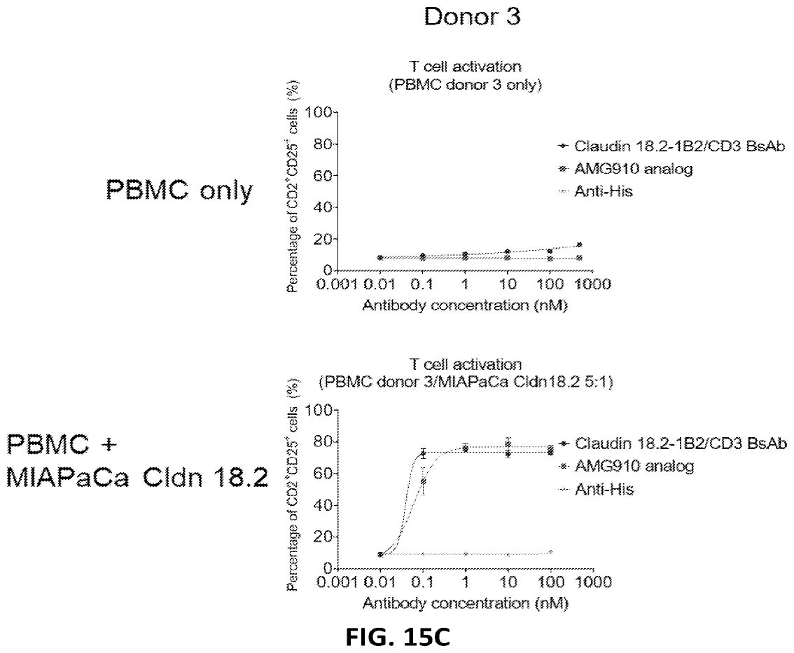

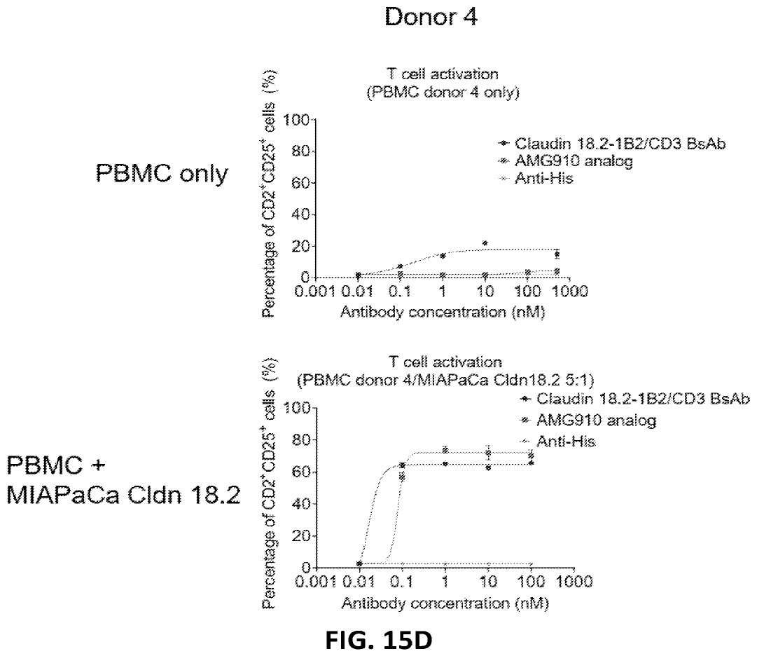

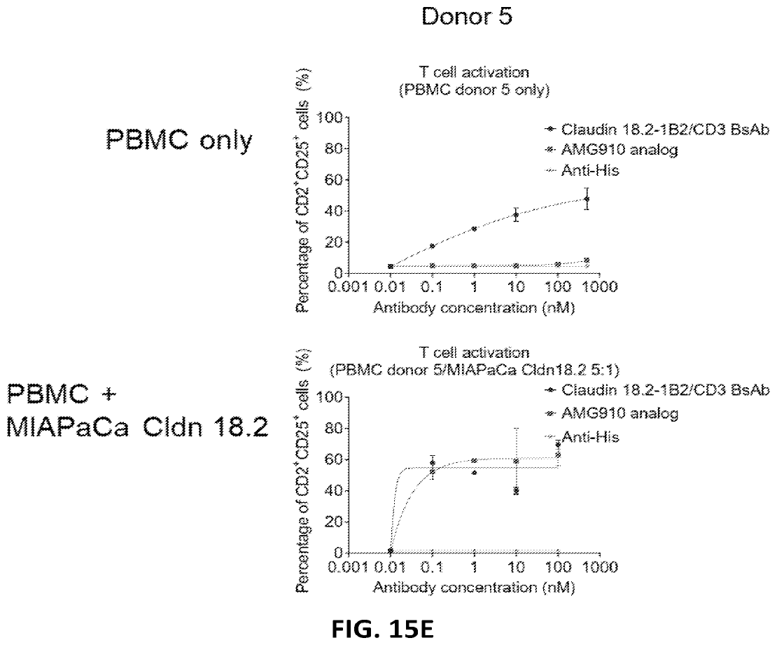

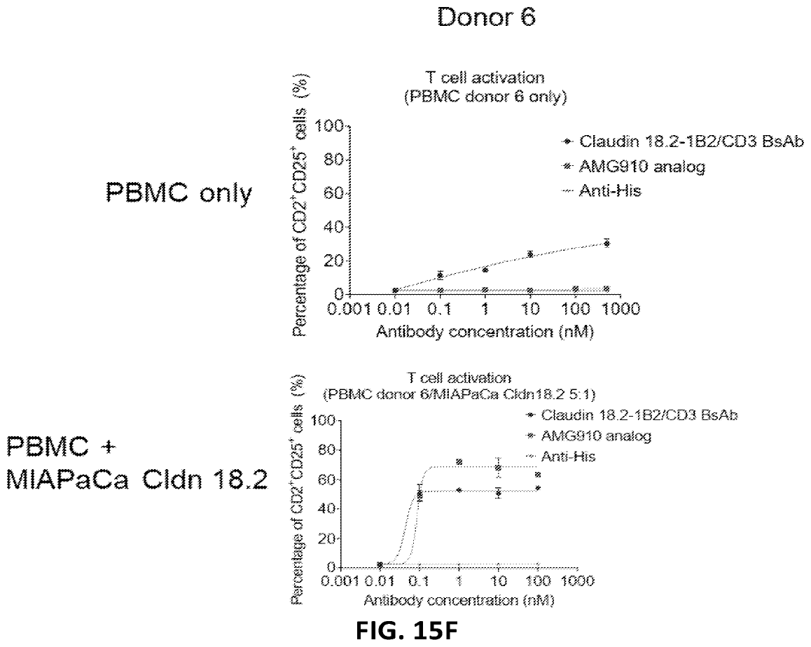

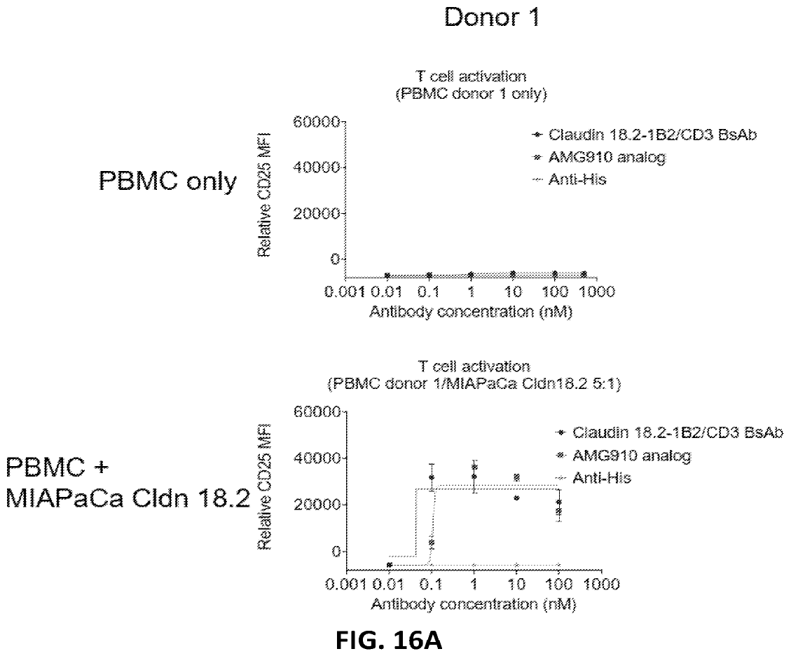

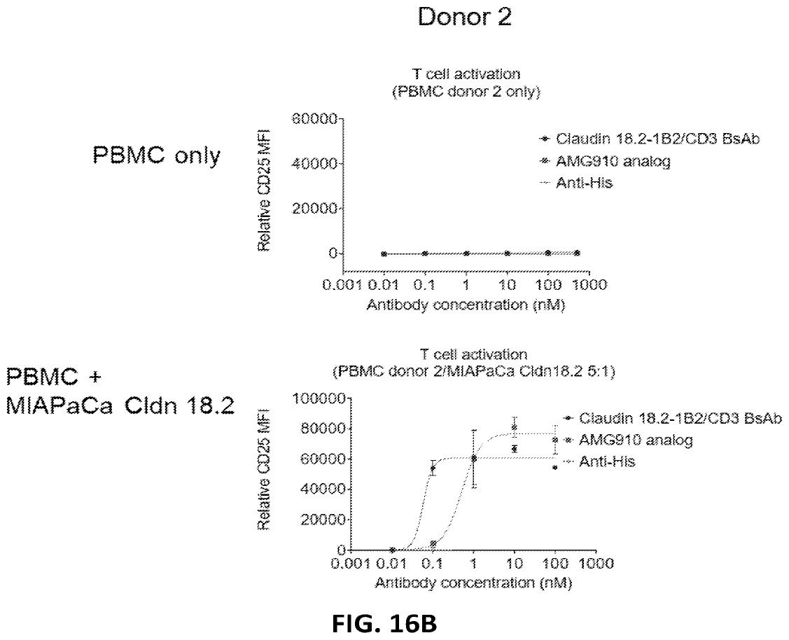

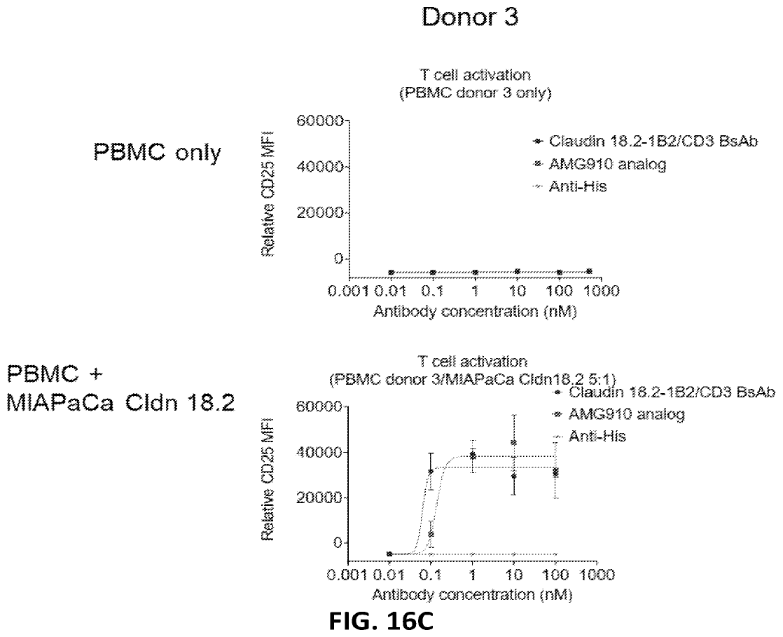

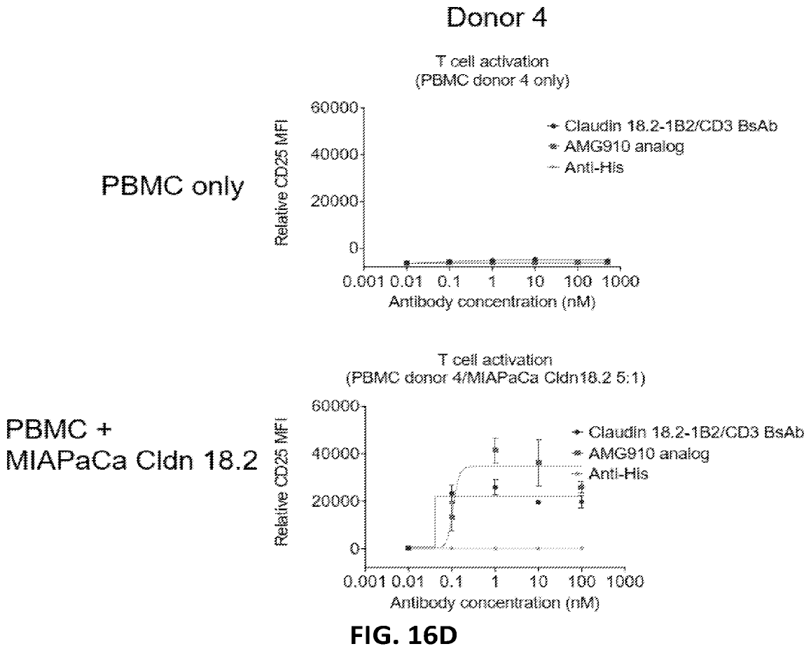

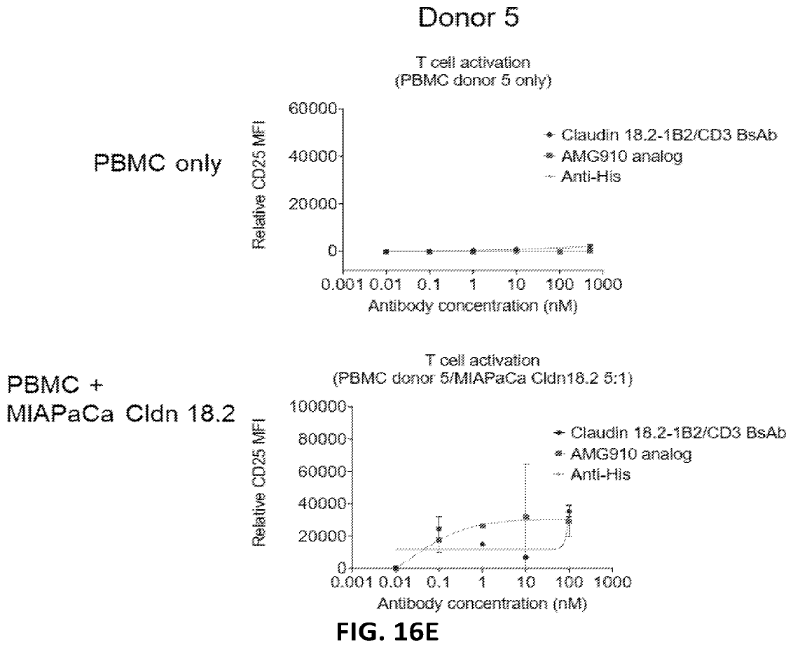

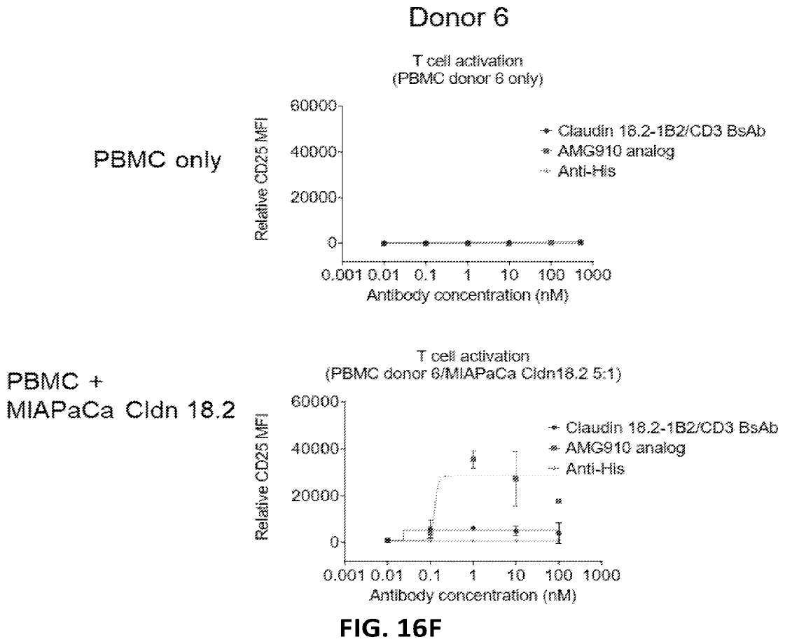

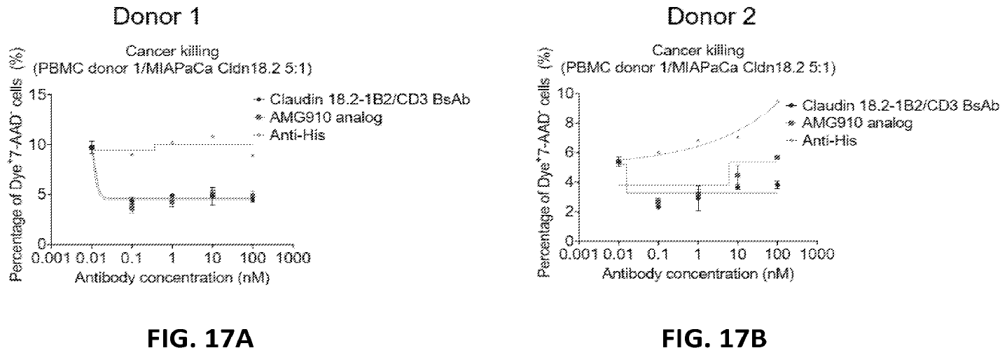

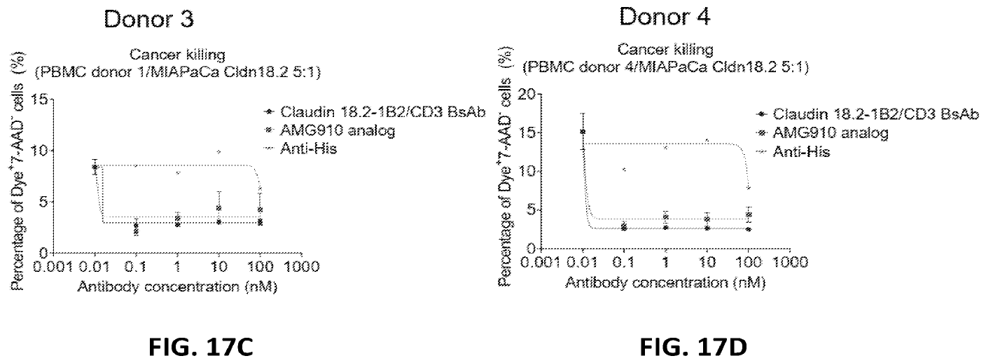

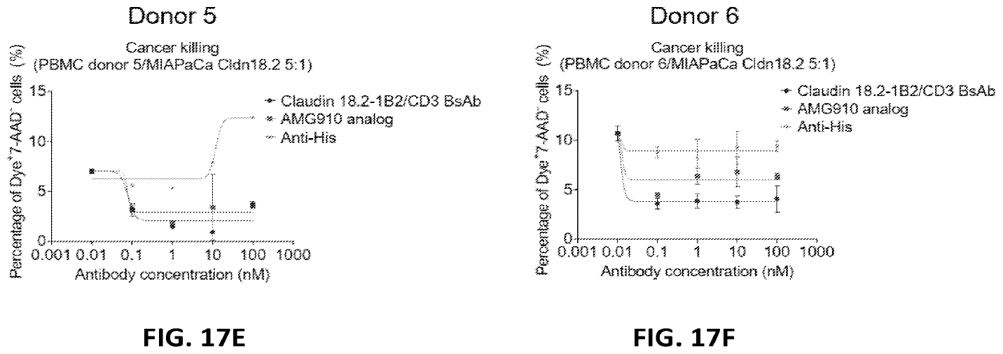

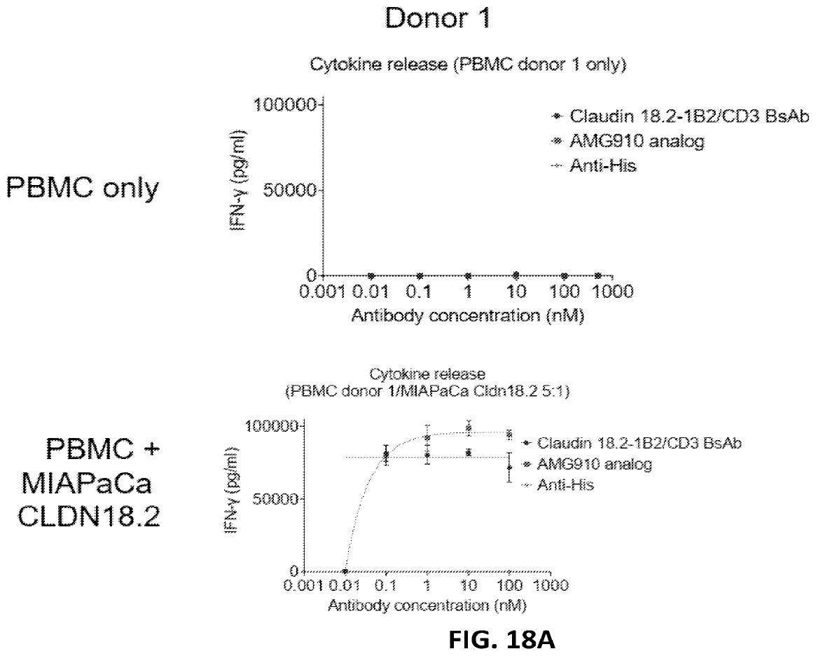

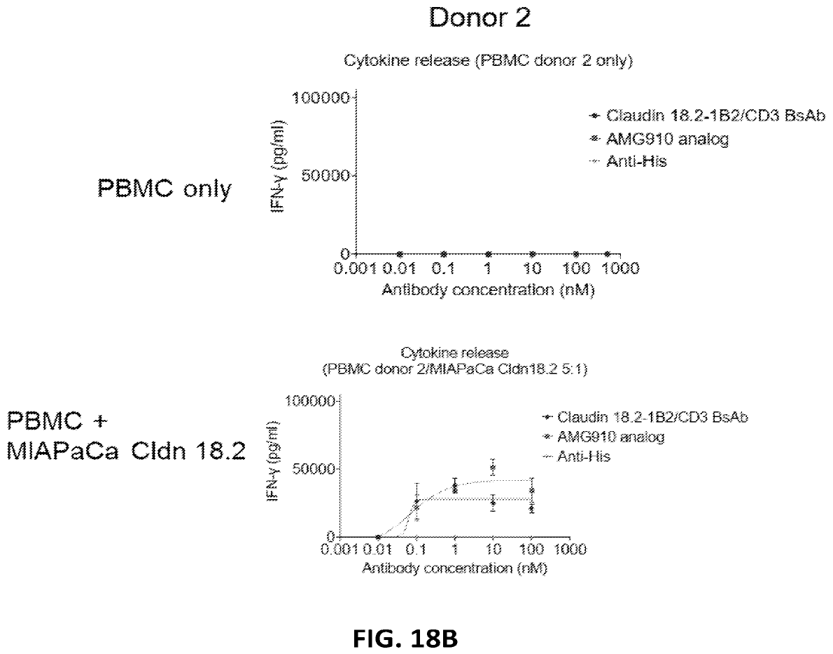

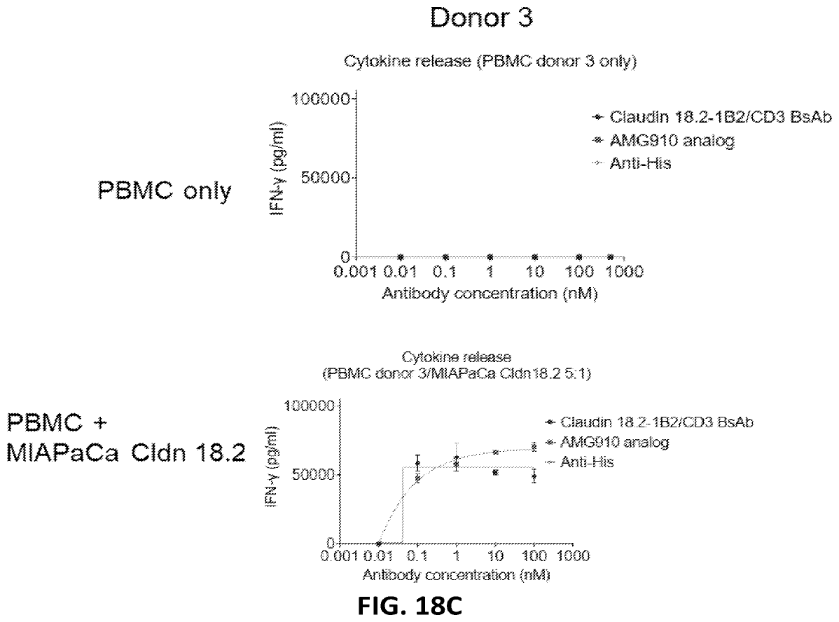

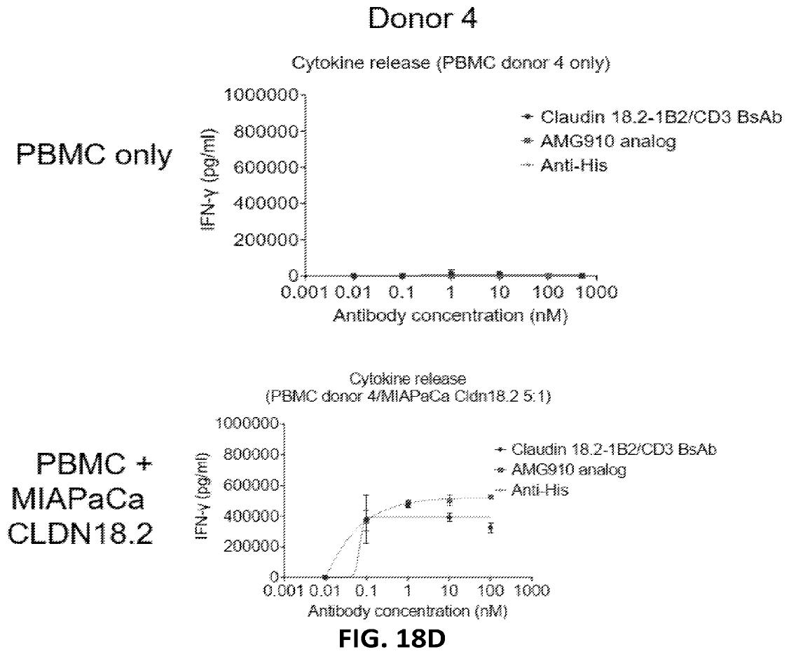

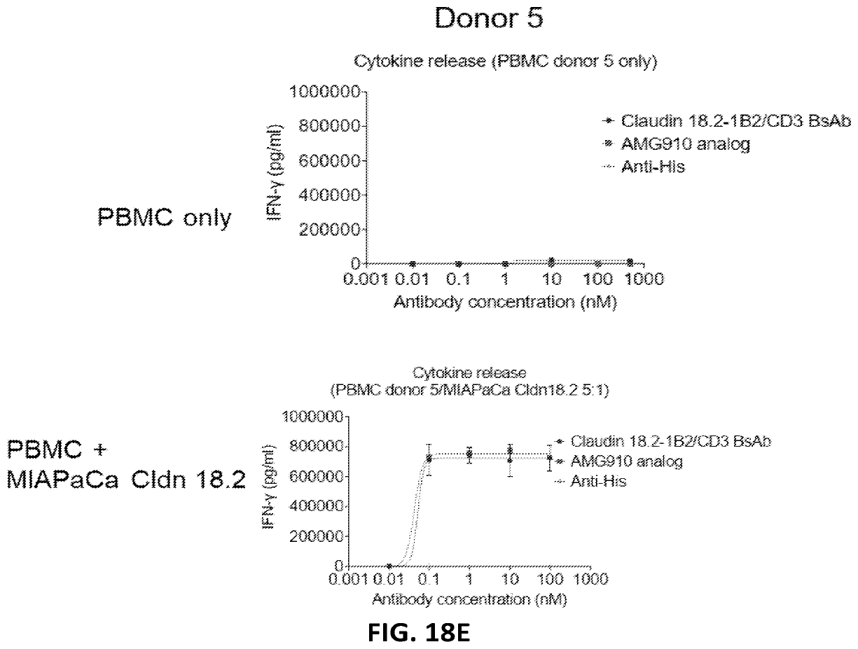

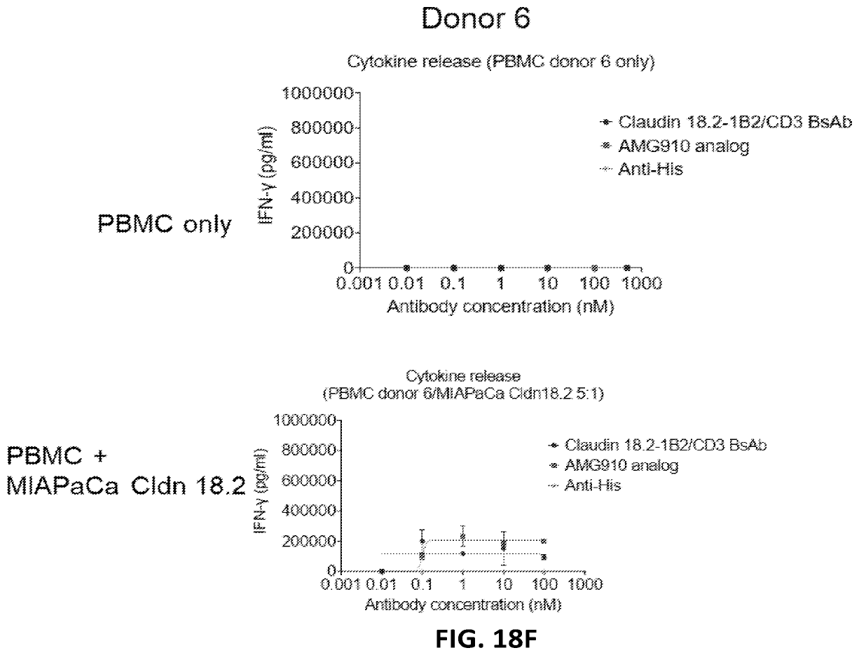

As shown in FIGS. 15A-15F and FIGS. 16A-16F, Claudin 18.2-1B2/CD3 BsAb showed a similar T cell activation effect as compared to that of AMG910 analog. As shown in FIGS. 17A-17F, Claudin 18.2-1B2/CD3 BsAb showed a similar cancer cell killing effect as compared to that of AMG910 analog. As shown in FIGS. 18A-18F, Claudin 18.2-1B2/CD3 BsAb showed a similar level of IFN-r release as compared to that of AMG910 analog, in the presence of the Claudin18.2-overexpressing MIA PaCa cells (target cells). The low levels of T cell activation and IFN-γ release in the absence of target cells indicate that Claudin 18.2-1B2/CD3 BsAb is generally safe for cancer treatment without inducing unspecific T cell response.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to Claudin 18.2, comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively; and
(4) the selected VHH CDRs 1, 2, 3 amino acid sequences are set forth in SEQ ID NOs: 10, 11, and 12, respectively.

2. The antibody or antigen-binding fragment thereof of claim 1, wherein the VHH comprises CDRs 1, 2, 3 with the amino acid sequences set forth in SEQ ID NOs: 1, 2, and 3, respectively.

3. The antibody or antigen-binding fragment thereof of claim 1, wherein the VHH comprises CDRs 1, 2, 3 with the amino acid sequences set forth in SEQ ID NOs: 4, 5, and 6, respectively.

4. The antibody or antigen-binding fragment thereof of claim 1, wherein the VHH comprises CDRs 1, 2, 3 with the amino acid sequences set forth in SEQ ID NOs: 7, 8, and 9, respectively.

5. The antibody or antigen-binding fragment thereof of claim 1, wherein the VHH comprises CDRs 1, 2, 3 with the amino acid sequences set forth in SEQ ID NOs: 10, 11, and 12, respectively.

6. An antibody or antigen-binding fragment thereof that binds to Claudin 18.2 comprising a heavy-chain antibody variable domain (VHH) comprising an amino acid sequence that is at least 80% identical to a selected VHH sequence, wherein the selected VHH sequence is selected from the group consisting of SEQ ID NOs: 19-22 and 91-94.

7. The antibody or antigen-binding fragment thereof of claim 6, wherein the VHH comprises the sequence of SEQ ID NO: 19, 20, 21, or 22.

8. The antibody or antigen-binding fragment thereof of claim 6, wherein the VHH comprises the sequence of SEQ ID NO: 21.

9. The antibody or antigen-binding fragment thereof of claim 6, wherein the VHH comprises the sequence of SEQ ID NO: 91 or 92.

10. The antibody or antigen-binding fragment thereof of claim 6, wherein the VHH comprises the sequence of SEQ ID NO: 93 or 94.

11. The antibody or antigen-binding fragment thereof of any one of claims 1-10, wherein the antibody or antigen-binding fragment specifically binds to Claudin 18.2.

12. The antibody or antigen-binding fragment thereof of any one of claims 1-11, wherein the antibody or antigen-binding fragment is a humanized antibody or antigen-binding fragment thereof.

13. An antibody or antigen-binding fragment thereof comprising the VHH CDRs 1, 2, 3, of the antibody or antigen-binding fragment thereof of any one of claims 1-12.

14. The antibody or antigen-binding fragment thereof of any one of claims 1-13, wherein the antibody or antigen-binding fragment comprises a human IgG Fc.

15. The antibody or antigen-binding fragment thereof of any one of claims 1-14, wherein the antibody or antigen-binding fragment comprises two or more heavy-chain antibody variable domains.

16. An antibody or antigen-binding fragment thereof that cross-competes with the antibody or antigen-binding fragment thereof of any one of claims 1-15.

17. An antibody or antigen-binding fragment thereof that binds to CD3 (cluster of differentiation 3) comprising:
a heavy chain variable region (VH) comprising complementarity determining regions (CDRs) 1, 2, and 3, wherein the VH CDR1 region comprises an amino acid sequence that is at least 80% identical to SEQ ID NO: 13, the VH CDR2 region comprises an amino acid sequence that is at least 80% identical to SEQ ID NO: 14, and the VH CDR3 region comprises an amino acid sequence that is at least 80% identical to SEQ ID NO: 15; and
a light chain variable region (VL) comprising CDRs 1, 2, and 3, wherein the VL CDR1 region comprises an amino acid sequence that is at least 80% identical to SEQ ID NO: 16, the VL CDR2 region comprises an amino acid sequence that is at least 80% identical to SEQ ID NO: 17, and the VL CDR3 region comprises an amino acid sequence that is at least 80% identical to SEQ ID NO: 18.

18. The antibody or antigen-binding fragment thereof of claim 17, wherein the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17, and 18, respectively.

19. An antibody or antigen-binding fragment thereof that binds to CD3 comprising
a heavy chain variable region (VH) comprising an amino acid sequence that is at least 90% identical to SEQ ID NO: 23 or 95, and a light chain variable region (VL) comprising an amino acid sequence that is at least 90% identical to SEQ ID NO: 24 or 96.

20. The antibody or antigen-binding fragment thereof of claim 19, wherein the VH comprises the sequence of SEQ ID NO: 23 or 95, and the VL comprises the sequence of SEQ ID NO: 24 or 96.

21. The antibody or antigen-binding fragment thereof of any one of claims 17-20, wherein the antibody or antigen-binding fragment specifically binds to human CD3.

22. The antibody or antigen-binding fragment thereof of any one of claims 17-21, wherein the antibody or antigen-binding fragment is a single-chain variable fragment (scFV).

23. An antibody or antigen-binding fragment thereof comprising the VH CDRs 1, 2, 3, and VL CDRs 1, 2, 3, of the antibody or antigen-binding fragment thereof of any one of claims 17-22.

24. An antibody or antigen-binding fragment thereof that cross-competes with the antibody or antigen-binding fragment thereof of any one of claims 17-23.

25. The antibody or antigen-binding fragment thereof of any one of claims 1-24, wherein the antibody is a bispecific antibody or a multispecific antibody.

26. A multi-specific antibody or antigen-binding fragment thereof, comprising a first antigen-binding site that specifically binds to CD3, and a second antigen-binding site that specifically binds to Claudin 18.2.

27. The multi-specific antibody or antigen-binding fragment thereof of claim 26, wherein the first antigen-binding site comprises a heavy chain variable region (VH) and a light chain variable region (VL), wherein the VH and the VL associate with each other and specifically bind to CD3, wherein
the heavy chain variable region (VH) comprising complementarity determining regions (CDRs) 1, 2, and 3, wherein the VH CDR1 region comprises an amino acid sequence that is at least 80% identical to SEQ ID NO: 13, the VH CDR2 region comprises an amino acid sequence that is at least 80% identical to SEQ ID NO: 14, and the VH CDR3 region comprises an amino acid sequence that is at least 80% identical to SEQ ID NO: 15; and
the light chain variable region (VL) comprising CDRs 1, 2, and 3, wherein the VL CDR1 region comprises an amino acid sequence that is at least 80% identical to SEQ ID NO: 16, the VL CDR2 region comprises an amino acid sequence that is at least 80% identical to SEQ ID NO: 17, and the VL CDR3 region comprises an amino acid sequence that is at least 80% identical to SEQ ID NO: 18.

28. The multi-specific antibody or antigen-binding fragment thereof of claim 27, wherein the heavy chain variable region (VH) comprises an amino acid sequence that is at least 90% identical to SEQ ID NO: 23 or 95, and a light chain variable region (VL) comprising an amino acid sequence that is at least 90% identical to SEQ ID NO: 24 or 96.

29. The multi-specific antibody or antigen-binding fragment thereof of any one of claims 26-28, wherein the second antigen-binding site specifically binds to Claudin 18.2, and the second antigen-binding site comprises a first heavy-chain antibody variable domain (VHH1) comprising complementarity determining regions (CDRs) 1, 2, and 3, wherein the VHH1 CDR1 region comprises an amino acid sequence that is at least 80% identical to a selected VHH1 CDR1 amino acid sequence, the VHH1 CDR2 region comprises an amino acid sequence that is at least 80% identical to a selected VHH1 CDR2 amino acid sequence, and the VHH1 CDR3 region comprises an amino acid sequence that is at least 80% identical to a selected VHH1 CDR3 amino acid sequence;
wherein the selected VHH1 CDRs 1, 2, and 3 amino acid sequences are one of the following:
(1) the selected VHH1 CDRs 1, 2, 3 amino acid sequences are set forth in SEQ ID NOs: 1, 2, and 3, respectively;
(2) the selected VHH1 CDRs 1, 2, 3 amino acid sequences are set forth in SEQ ID NOs: 4, 5, and 6, respectively;
(3) the selected VHH1 CDRs 1, 2, 3 amino acid sequences are set forth in SEQ ID NOs: 7, 8, and 9, respectively; and
(4) the selected VHH1 CDRs 1, 2, 3 amino acid sequences are set forth in SEQ ID NOs: 10, 11, and 12, respectively.

30. The multi-specific antibody or antigen-binding fragment thereof of claim 29, wherein the first heavy-chain antibody variable domain (VHH1) comprises an amino acid sequence that is at least 80% identical to a selected VHH1 sequence, wherein the selected VHH1 sequence is selected from the group consisting of SEQ ID NOs: 19-22 and 91-94.

31. The multi-specific antibody or antigen-binding fragment thereof of any one of claims 26-30, further comprising a third antigen-binding site that specifically binds to Claudin 18.2.

32. The multi-specific antibody or antigen-binding fragment thereof of claim 31, wherein the third antigen-binding site specifically binds to Claudin 18.2, and the third antigen-binding site comprises a second heavy-chain antibody variable domain (VHH2) comprising complementarity determining regions (CDRs) 1, 2, and 3, wherein the VHH2 CDR1 region comprises an amino acid sequence that is at least 80% identical to a selected VHH2 CDR1 amino acid sequence, the VHH2 CDR2 region comprises an amino acid sequence that is at least 80% identical to a selected VHH2 CDR2 amino acid sequence, and the VHH2 CDR3 region comprises an amino acid sequence that is at least 80% identical to a selected VHH2 CDR3 amino acid sequence;
wherein the selected VHH2 CDRs 1, 2, and 3 amino acid sequences are one of the following:
(1) the selected VHH2 CDRs 1, 2, 3 amino acid sequences are set forth in SEQ ID NOs: 1, 2, and 3, respectively;
(2) the selected VHH2 CDRs 1, 2, 3 amino acid sequences are set forth in SEQ ID NOs: 4, 5, and 6, respectively;
(3) the selected VHH2 CDRs 1, 2, 3 amino acid sequences are set forth in SEQ ID NOs: 7, 8, and 9, respectively; and
(4) the selected VHH2 CDRs 1, 2, 3 amino acid sequences are set forth in SEQ ID NOs: 10, 11, and 12, respectively.

33. The multi-specific antibody or antigen-binding fragment thereof of claim 32, wherein the second heavy-chain antibody variable domain (VHH2) comprises an amino acid sequence that is at least 80% identical to a selected VHH2 sequence, wherein the selected VHH2 sequence is selected from the group consisting of SEQ ID NOs: 19-22 and 91-94.

34. The multi-specific antibody or antigen-binding fragment thereof of any one of claims 26-33, wherein the VH and the VL are linked by a linker peptide sequence to form an scFv.

35. The multi-specific antibody or antigen-binding fragment thereof of claim 34, wherein the linker peptide sequence comprises a sequence that is at least 80% identical to any one of SEQ ID NOs: 35-40.

36. A polypeptide complex, comprising
(a) a first polypeptide comprising from N-terminus to C-terminus: a first heavy-chain antibody variable domain (VHH), a first hinge region, a first Fc region;
(b) a second polypeptide comprising from N-terminus to C-terminus: a heavy chain variable region (VH), a second hinge region, and a second Fc region; and
(c) a third polypeptide comprising a light chain variable region (VL);
wherein the first VHH specifically binds to Claudin 18.2, wherein the VH and the VL associate with each other and specifically bind to CD3.

37. The polypeptide complex of claim 36, wherein the first hinge region and/or the second hinge region comprise a sequence that is at least 80% identical to any one of SEQ ID NOs: 25-29.

38. The polypeptide complex of claim 36 or 37, wherein the first Fc region and/or the second Fc region comprise a sequence that is at least 80% identical to SEQ ID NO: 30 or 31.

39. The polypeptide complex of any one of claims 36-38, wherein the first polypeptide comprises a sequence that is at least 80% identical to SEQ ID NO: 41,42, 75, or 76; wherein the second polypeptide comprises a sequence that is at least 80% identical to SEQ ID NO: 43 or 44; and/or wherein the third polypeptide comprises a sequence that is at least 80% identical to SEQ ID NO: 45.

40. The polypeptide complex of any one of claims 36-39, wherein the first polypeptide further comprises a second VHH that specifically binds to Claudin 18.2, wherein the second VHH is linked to the N-terminus of the first VHH via a linker peptide sequence.

41. The polypeptide complex of claim 40, wherein the linker peptide sequence comprises a sequence that is at least 80% identical to any one of SEQ ID NOs: 35-40.

42. The polypeptide complex of claim 40 or 41, wherein the first polypeptide comprises a sequence that is at least 80% identical to SEQ ID NO: 46,47, 77, or 78; wherein the second polypeptide comprises a sequence that is at least 80% identical to SEQ ID NO: 48 or 49; and/or wherein the third polypeptide comprises a sequence that is at least 80% identical to SEQ ID NO: 50.

43. A polypeptide complex, comprising
(a) a first polypeptide comprising from N-terminus to C-terminus: a first heavy-chain antibody variable domain (VHH), a first hinge region, a first Fc region; and
(b) a second polypeptide comprising from N-terminus to C-terminus: a single-chain variable fragment (scFv), a second hinge region, and a second Fc region;
wherein the first VHH specifically binds to Claudin 18.2; wherein the scFv comprises a heavy chain variable region (VH), a first linker peptide sequence, and a light chain variable region (VL); wherein the VH and the VL associate with each other and specifically bind to CD3.

44. The polypeptide complex of claim 43, wherein the first linker peptide sequence comprises a sequence that is at least 80% identical to any one of SEQ ID NOs: 35-40.

45. The polypeptide complex of claim 43 or 44, wherein the first hinge region and/or the second hinge region comprise a sequence that is at least 80% identical to any one of SEQ ID NOs: 25-29.

46. The polypeptide complex of any one of claims 43-45, wherein the first Fc region and/or the second Fc region comprise a sequence that is at least 80% identical to SEQ ID NO: 30 or 31.

47. The polypeptide complex of any one of claims 43-46, wherein the first polypeptide comprises a sequence that is at least 80% identical to SEQ ID NO: 51,52, 79, or 80; and/or wherein the second polypeptide comprises a sequence that is at least 80% identical to SEQ ID NO: 53 or 54.

48. The polypeptide complex of any one of claims 43-47, wherein the first polypeptide further comprises a second VHH that specifically binds to Claudin 18.2, wherein the second VHH is linked to the N-terminus of the first VHH via a second linker peptide sequence.

49. The polypeptide complex of claim 48, wherein the second linker peptide sequence comprises a sequence that is at least 80% identical to any one of SEQ ID NOs: 35-40.

50. The polypeptide complex of claim 48 or 49, wherein the first polypeptide comprises a sequence that is at least 80% identical to SEQ ID NO: 55,56, 81, or 82; and/or wherein the second polypeptide comprises a sequence that is at least 80% identical to SEQ ID NO: 57 or 58.

51. A polypeptide complex, comprising
(a) a first polypeptide comprising from N-terminus to C-terminus: a first heavy chain variable region (VH1), a first hinge region, a first Fc region; a first linker peptide sequence, and a first heavy-chain antibody variable domain (VHH1);
(b) a second polypeptide comprising a first light chain variable region (VL1);
(c) a third polypeptide comprising from N-terminus to C-terminus: a second heavy chain variable region (VH2), a second hinge region, a second Fc region, a second linker peptide sequence, and a second heavy-chain antibody variable domain (VHH2); and
(d) a fourth polypeptide comprising a second light chain variable region (VL2);
wherein the VHH1 and/or the VHH2 specifically bind to Claudin 18.2; wherein the VH1 and the VL1 associate with each other and specifically bind to CD3; wherein the VH2 and the VL2 associate with each other and specifically bind to CD3.

52. The polypeptide complex of claim 51, wherein the first linker peptide sequence and/or the second linker peptide sequence comprises a sequence that is at least 80% identical to any one of SEQ ID NOs: 35-40.

53. The polypeptide complex of claim 50 or 51, wherein the first hinge region and/or the second hinge regions comprise a sequence that is at least 80% identical to any one of SEQ ID NOs: 25-29.

54. The polypeptide complex of any one of claims 51-53, wherein sequences of the VHH1 and the VHH2 are identical.

55. The polypeptide complex of claim 54, wherein the first Fc region and/or the second Fc region comprise a sequence that is at least 80% identical to SEQ ID NO: 32.

56. The polypeptide complex of claim 54 or 55, wherein the first polypeptide comprises a sequence that is at least 80% identical to SEQ ID NO: 59,60, 83, or 84; wherein the second polypeptide comprises a sequence that is at least 80% identical to SEQ ID NO: 61; wherein the third polypeptide comprises a sequence that is at least 80% identical to SEQ ID NO: 59,60, 83, or 84; and/or wherein the fourth polypeptide comprises a sequence that is at least 80% identical to SEQ ID NO: 61.

57. The polypeptide complex of any one of claims 51-53, wherein sequences of the VHH1 and the VHH2 are different.

58. The polypeptide complex of claim 57, wherein the first Fc region and/or the second Fc region comprise a sequence that is at least 80% identical to SEQ ID NO: 30 or 31.

59. The polypeptide complex of claim 57 or 58, wherein the first polypeptide comprises a sequence that is at least 80% identical to SEQ ID NO: 62,63, 85, or 86; wherein the second polypeptide comprises a sequence that is at least 80% identical to SEQ ID NO: 66; wherein the third polypeptide comprises a sequence that is at least 80% identical to SEQ ID NO: 64 or 65; and/or wherein the fourth polypeptide comprises a sequence that is at least 80% identical to SEQ ID NO: 66.

60. A polypeptide complex, comprising
(a) a first polypeptide comprising from N-terminus to C-terminus: a first heavy-chain antibody variable domain (VHH1), a first linker peptide sequence, a first heavy chain variable region (VH1), a first hinge region, and a first Fc region;
(b) a second polypeptide comprising a first light chain variable region (VL1);
(c) a third polypeptide comprising from N-terminus to C-terminus: a second heavy-chain antibody variable domain (VHH2), a second linker peptide sequence, a second heavy chain variable region (VH2), a second hinge region, and a second Fc region; and
(d) a fourth polypeptide comprising a second light chain variable region (VL2);
wherein the VHH1 and the VHH2 specifically bind to Claudin 18.2; wherein the VH1 and the VL1 associate with each other and specifically bind to CD3; wherein the VH2 and the VL2 associate with each other and specifically bind to CD3.

61. The polypeptide complex of claim 60, wherein the first linker peptide sequence and/or the second linker peptide sequence comprises a sequence that is at least 80% identical to any one of SEQ ID NOs: 35-40.

62. The polypeptide complex of claim 59 or 60, wherein the first hinge region and/or the second hinge regions comprise a sequence that is at least 80% identical to any one of SEQ ID NOs: 25-29.

63. The polypeptide complex of any one of claims 59-62, wherein sequences of the VHH1 and the VHH2 are identical.

64. The polypeptide complex of claim 63, wherein the first Fc region and/or the second Fc region comprise a sequence that is at least 80% identical to SEQ ID NO: 32.

65. The polypeptide complex of claim 63 or 64, wherein the first polypeptide comprises a sequence that is at least 80% identical to SEQ ID NO: 67,68, 87, or 88; wherein the second polypeptide comprises a sequence that is at least 80% identical to SEQ ID NO: 69; wherein the third polypeptide comprises a sequence that is at least 80% identical to SEQ ID NO: 67, 68, 87, or 88; and/or wherein the fourth polypeptide comprises a sequence that is at least 80% identical to SEQ ID NO: 69.

66. The polypeptide complex of any one of claims 59-62, wherein sequences of the VHH1 and the VHH2 are different.

67. The polypeptide complex of claim 66, wherein the first Fc region and/or the second Fc region comprise a sequence that is at least 80% identical to SEQ ID NO: 30 or 31.

68. The polypeptide complex of claim 66 or 67, wherein the first polypeptide comprises a sequence that is at least 80% identical to SEQ ID NO: 70,71, 89, or 90; wherein the second polypeptide comprises a sequence that is at least 80% identical to SEQ ID NO: 74; wherein the third polypeptide comprises a sequence that is at least 80% identical to SEQ ID NO: 72 or 73; and/or wherein the fourth polypeptide comprises a sequence that is at least 80% identical to SEQ ID NO: 74.

69. A nucleic acid comprising a polynucleotide encoding the antibody or antigen-binding fragment thereof of any one of claims 1-25, the multi-specific antibody or antigen-binding fragment thereof of any one of claims 26-35, or the polypeptide complex of any one of claims 36-68.

70. The nucleic acid of claim 69, wherein the nucleic acid is a DNA (e.g., cDNA) or RNA (e.g., mRNA).

71. A vector comprising one or more of the nucleic acids of claim 69 or 70.

72. A cell comprising the vector of claim 71.

73. The cell of claim 72, wherein the cell is a CHO cell.

74. A cell comprising one or more of the nucleic acids of claim 69 or 70.

75. A method of producing an antibody or an antigen-binding fragment thereof, the method comprising
(a) culturing the cell of any one of claims 72-74 under conditions sufficient for the cell to produce the antibody or the antigen-binding fragment; and
(b) collecting the antibody or the antigen-binding fragment produced by the cell.

76. An antibody-drug conjugate comprising the antibody or antigen-binding fragment thereof of any one of claims 1-25, or the multi-specific antibody or antigen-binding fragment thereof of any one of claims 26-35, covalently bound to a therapeutic agent.

77. The antibody drug conjugate of claim 76, wherein the therapeutic agent is a cytotoxic or cytostatic agent.

78. A method of treating a subject having cancer, the method comprising administering a therapeutically effective amount of a composition comprising the antibody or antigen-binding fragment thereof of any one of claims 1-25, the multi-specific antibody or antigen-binding fragment thereof of any one of claims 26-35, the polypeptide complex of any one of claims 36-68, or the antibody-drug conjugate of claim 76 or 77, to the subject.

79. The method of claim 78, wherein the subject has a cancer expressing Claudin 18.2.

80. The method of claim 78 or 79, wherein the cancer is a solid tumor.

81. The method of claim 78 or 79, wherein the cancer is gastric or pancreatic adenocarcinoma.

82. A method of decreasing the rate of tumor growth, the method comprising
contacting a tumor cell with an effective amount of a composition comprising the antibody or antigen-binding fragment thereof of any one of claims 1-25, the multi-specific antibody or antigen-binding fragment thereof of any one of claims 26-35, the polypeptide complex of any one of claims 36-68, or the antibody-drug conjugate of claim 76 or 77.

83. A method of killing a tumor cell, the method comprising
contacting a tumor cell with an effective amount of a composition comprising the antibody or antigen-binding fragment thereof of any one of claims 1-25, the multi-specific antibody or antigen-binding fragment thereof of any one of claims 26-35, the polypeptide complex of any one of claims 36-68, or the antibody-drug conjugate of claim 76 or 77.

84. A method of killing a tumor cell, the method comprising
contacting a tumor cell with an effective amount of a composition comprising the antibody or antigen-binding fragment thereof of any one of claims 1-25, the multi-specific antibody or antigen-binding fragment thereof of any one of claims 26-35, the polypeptide complex of any one of claims 36-68, or the antibody-drug conjugate of claim 76 or 77.

85. A pharmaceutical composition comprising the antibody or antigen-binding fragment thereof of any one of claims 1-25, the multi-specific antibody or antigen-binding fragment thereof of any one of claims 26-35, or the polypeptide complex of any one of claims 36-68, and a pharmaceutically acceptable carrier.

86. A pharmaceutical composition comprising the antibody-drug conjugate of claim 76 or 77, and a pharmaceutically acceptable carrier.
